SEQUENCE LISTING (61 sequences)

SEQ ID NO: 1 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
agagcaacagugcuguggccguuuuagagcuagaaauagcaaguuaaaauaaggcuaguccguuaucaacuugaaaaaguggcaccgagucggugcuuuu

SEQ ID NO: 2 - RNA, 100 nt, Artificial Sequence; identical to SEQ ID NO: 1
  Synthetic

SEQ ID NO: 3 - RNA, 20 nt, Artificial Sequence
  Synthetic
  misc_feature (1)..(4): modified with 2′-O-methyl phosphorothioate
agagcaacagugcuguggcc

SEQ ID NO: 4 - RNA, 20 nt, Artificial Sequence
  Synthetic
agagcaacagugcuguggcc

SEQ ID NO: 5 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcuacucucucuuucuggccguuuuagagcuagaaauagcaaguuaaaauaaggcuaguccguuaucaacuugaaaaaguggcaccgagucggugcuuuu

SEQ ID NO: 6 - RNA, 100 nt, Artificial Sequence; identical to SEQ ID NO: 5
  Synthetic

SEQ ID NO: 7 - RNA, 20 nt, Artificial Sequence
  Synthetic
  misc_feature (1)..(4): modified with 2′-O-methyl phosphorothioate
gcuacucucucuuucuggcc

SEQ ID NO: 8 - RNA, 20 nt, Artificial Sequence
  Synthetic
gcuacucucucuuucuggcc

SEQ ID NO: 9 - DNA, 23 bp, Artificial Sequence
  Synthetic
agagcaacagtgctgtggcctgg

SEQ ID NO: 10 - DNA, 20 bp, Artificial Sequence
  Synthetic
agagcaacagtgctgtggcc

SEQ ID NO: 11 - DNA, 23 bp, Artificial Sequence
  Synthetic
gctactctctctttctggcctgg

SEQ ID NO: 12 - DNA, 20 bp, Artificial Sequence
  Synthetic
gctactctctctttctggcc

SEQ ID NO: 13 - DNA, 19 bp, Artificial Sequence
  Synthetic
aagagcaacaaatctgact

SEQ ID NO: 14 - DNA, 39 bp, Artificial Sequence
  Synthetic
aagagcaacagtgctgtgcctggagcaacaaatctgact

SEQ ID NO: 15 - DNA, 33 bp, Artificial Sequence
  Synthetic
aagagcaacagtgctggagcaacaaatctgact

SEQ ID NO: 16 - DNA, 34 bp, Artificial Sequence
  Synthetic
aagagcaacagtgcctggagcaacaaatctgact

SEQ ID NO: 17 - DNA, 19 bp, Artificial Sequence
  Synthetic
aagagcaacagtgctgact

SEQ ID NO: 18 - DNA, 41 bp, Artificial Sequence
  Synthetic
aagagcaacagtgctgtgggcctggagcaacaaatctgact

SEQ ID NO: 19 - DNA, 38 bp, Artificial Sequence
  Synthetic
aagagcaacagtgctggcctggagcaacaaatctgact

SEQ ID NO: 20 - DNA, 41 bp, Artificial Sequence
  Synthetic
aagagcaacagtgctgtgtgcctggagcaacaaatctgact

SEQ ID NO: 21 - DNA, 79 bp, Artificial Sequence
  Synthetic
cgtggccttagctgtgctcgcgctactctctctttctgcctggaggctatccagcgtgagtctctcctaccctcccgct

SEQ ID NO: 22 - DNA, 78 bp, Artificial Sequence
  Synthetic
cgtggccttagctgtgctcgcgctactctctctttcgcctggaggctatccagcgtgagtctctcctaccctcccgct

SEQ ID NO: 23 - DNA, 75 bp, Artificial Sequence
  Synthetic
cgtggccttagctgtgctcgcgctactctctctttctggaggctatccagcgtgagtctctcctaccctcccgct

SEQ ID NO: 24 - DNA, 84 bp, Artificial Sequence
  Synthetic
cgtggccttagctgtgctcgcgctactctctctttctggatagcctggaggctatccagcgtgagtctctcctaccctcccgct

SEQ ID NO: 25 - DNA, 55 bp, Artificial Sequence
  Synthetic
cgtggccttagctgtgctcgcgctatccagcgtgagtctctcctaccctcccgct

SEQ ID NO: 26 - DNA, 82 bp, Artificial Sequence
  Synthetic
cgtggccttagctgtgctcgcgctactctctctttctgtggcctggaggctatccagcgtgagtctctcctaccctcccgct

SEQ ID NO: 27 - DNA, 4688 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 28 - DNA, 130 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 29 - DNA, 141 bp, Artificial Sequence
  Synthetic
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 30 - DNA, 4364 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 31 - DNA, 800 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 32 - DNA, 804 bp, Artificial Sequence
  Synthetic
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 33 - DNA, 1524 bp, Artificial Sequence
  Synthetic
atggcgcttccggtgacagcactgctcctccccttggcgctgttgctccacgcagcaaggccgcaggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 34 - DNA, 735 bp, Artificial Sequence
  Synthetic
caggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaa

SEQ ID NO: 35 - DNA, 126 bp, Artificial Sequence
  Synthetic
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 36 - DNA, 120 bp, Artificial Sequence
  Synthetic
tcaaagcggagtaggttgttgcattccgattacatgaatatgactcctcgccggcctgggccgacaagaaaacattaccaaccctatgcccccccacgagacttcgctgcgtacaggtcc

SEQ ID NO: 37 - DNA, 336 bp, Artificial Sequence
  Synthetic
cgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 38 - DNA, 1178 bp, Artificial Sequence
  Synthetic
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 39 - DNA, 49 bp, Artificial Sequence
  Synthetic
aataaaatcgctatccatcgaagatggatgtgtgttggttttttgtgtg

SEQ ID NO: 40 - protein, 508 aa, Artificial Sequence
  Synthetic
MALPVTALLLPLALLLHAARPQVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 245 aa, Artificial Sequence
  Synthetic
QVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIK

SEQ ID NO: 42 - protein, 118 aa, Artificial Sequence
  Synthetic
QVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSS

SEQ ID NO: 43 - protein, 112 aa, Artificial Sequence
  Synthetic
EIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIK

SEQ ID NO: 44 - protein, 16 aa, Artificial Sequence
  Synthetic
RASQSLVHSNGNTHLH

SEQ ID NO: 45 - protein, 5 aa, Artificial Sequence
  Synthetic
SVSNR

SEQ ID NO: 46 - protein, 9 aa, Artificial Sequence
  Synthetic
SQTSHIPYT

SEQ ID NO: 47 - protein, 5 aa, Artificial Sequence
  Synthetic
NYVIH

SEQ ID NO: 48 - protein, 17 aa, Artificial Sequence
  Synthetic
YILPYNDLTKYSQKFQG

SEQ ID NO: 49 - protein, 9 aa, Artificial Sequence
  Synthetic
WDWDGFFDP

SEQ ID NO: 50 - protein, 7 aa, Artificial Sequence
  Synthetic
GNTLTNY

SEQ ID NO: 51 - protein, 6 aa, Artificial Sequence
  Synthetic
LPYNDL

SEQ ID NO: 52 - protein, 9 aa, Artificial Sequence
  Synthetic
WDWDGFFDP

SEQ ID NO: 53 - protein, 15 aa, Artificial Sequence
  Synthetic
GGGGSGGGGSGGGGS

SEQ ID NO: 54 - protein, 22 aa, Artificial Sequence
  Synthetic
MLLLVTSLLLCELPHPAFLLIP

SEQ ID NO: 55 - protein, 21 aa, Artificial Sequence
  Synthetic
MALPVTALLLPLALLLHAARP

SEQ ID NO: 56 - protein, 23 aa, Artificial Sequence
  Synthetic
IYIWAPLAGTCGVLLLSLVITLY

SEQ ID NO: 57 - protein, 42 aa, Artificial Sequence
  Synthetic
KRGRKKLLYIFKQPFMRPVQTTQEEDGCSCRFPEEEEGGCEL

SEQ ID NO: 58 - protein, 40 aa, Artificial Sequence
  Synthetic
SKRSRLLHSDYMNMTPRRPGPTRKHYQPYAPPRDFAAYRS

SEQ ID NO: 59 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 84 aa, Artificial Sequence
  Synthetic
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 61 - protein, 1368 aa, Artificial Sequence [sequence omitted]

(19) United States
(12) Patent Application Publication
(10) Pub. No.: US 2023/0220059 A1
(43) Pub. Date: Jul. 13, 2023

(54) GENETICALLY ENGINEERED T CELLS EXPRESSING BCMA-SPECIFIC CHIMERIC ANTIGEN RECEPTORS AND USES THEREOF IN CANCER THERAPY

(71) Applicant: CRISPR THERAPEUTICS AG, Zug (CH)

(72) Inventors: Jonathan Alexander TERRETT, Cambridge, MA (US); Ewelina MORAWA, Cambridge, MA (US); Jason SAGERT, Cambridge, MA (US); Annie Yang WEAVER, Cambridge, MA (US)

(21) Appl. No.: 17/758,947

(22) PCT Filed: Jan. 15, 2021
(86) PCT No.: PCT/IB2021/050305

§ 371 Date: Jul. 15, 2022

Related U.S. Application Data
(60) Provisional application No. 62/962,315, filed on Jan. 17, 2020.
(60) Provisional application No. 63/013,587, filed on Apr. 22, 2020.
(60) Provisional application No. 63/129,973, filed on Dec. 23, 2020.

(51) Int. Cl. C07K 16/24; A61K 35/17; C07K 16/28; C12N 15/113

(52) U.S. Cl. CPC C07K 16/241; A61K 35/17; C07K 16/2815; C12N 15/1138; A61K 2039/505

(57) ABSTRACT

Genetically engineered T cells expressing a chimeric antigen receptor (CAR) that binds B-cell maturation antigen (BCMA) and uses thereof for treating multiple myeloma, for example, refractory and/or relapsed multiple myeloma. The genetically engineered T cells may comprise a disrupted endogenous TRAC gene and/or a disrupted endogenous β2M gene.

### CROSS-REFERENCE TO RELATED APPLICATIONS

This application claims the benefit of priority to U.S. Provisional Patent Application No. 62/962,315, filed Jan. 17, 2020, U.S. Provisional Patent Application No. 63/013,587, filed Apr. 22, 2020, and U.S. Provisional Patent Application No. 63/129,973, filed Dec. 23, 2020. The entire content of each of the prior applications is hereby incorporated by reference in its entirety.

### BACKGROUND OF THE INVENTION

Multiple myeloma (MM) is a malignancy of terminally differentiated plasma cells in the bone marrow. MM results from the secretion of a monoclonal immunoglobulin protein (also known as M-protein or monoclonal protein) or monoclonal free light chains by abnormal plasma cells, and is differentiated on the spectrum of plasma cell dyscrasias by characteristic bone marrow biopsy findings as well as symptoms attributable to end organ damage related to plasma cell proliferation (hypercalcemia, renal insufficiency, anemia, fractures) (Kumar 2017a). MM represents about 10% of all hematologic malignancies and is the second most common hematologic malignancy after Non-Hodgkin lymphoma (NHL) (Kumar 2017a, Rajkumar and Kumar 2016). For most patients, MM is an incurable disease that ultimately leads to death. There is an unmet need for effective therapies for treating MM, particularly relapsed/refractory MM.

### SUMMARY OF THE INVENTION

The present disclosure is based, at least in part, on the development of an immune cell therapy involving allogenic T cells comprising disrupted endogenous TRAC and β2M genes and expressing a chimeric antigen receptor (CAR) targeting B-cell maturation antigen (BCMA). The allogenic anti-BCMA CAR-T cells resulted in eradication of human multiple myeloma tumors (carrying BCMA positive tumor cells) as observed in xenograft mouse models. Significantly, it has been observed that administration of the allogeneic anti-BCMA CAR-T cells eliminated tumor burden and protected animals from re-challenge with tumors cells. Further, allogenic the anti-BCMA CAR-T cells showed high selectivity against BCMA⁺ cells and did not result in undesirable oncogenic transformation. In addition, data from an animal model showed that the allogenic anti-BCMA CAR-T cells did not induce graft versus host disease (GvHD) or host versus graft disease (HvGD). Taken together, the anti-BCMA allogenic CAR-T cells are expected to be highly effective and safe in therapeutic uses (e.g., cancer treatment) in human subjects.

Accordingly, the present disclosure provides compositions comprising genetically engineered T cells that have disrupted endogenous TRAC and β2M genes and express a chimeric antigen receptor (CAR) specific for B cell maturation antigen (BCMA) and allogenic anti-BCMA CAR-T cell therapy for treating cancer (e.g., MM). The genetically engineered T cells may be derived from one or more healthy donors (e.g., healthy human donors).

In some aspects, the present disclosure provides a population of genetically engineered T cells, which comprise T cells comprising a nucleic acid comprising a nucleotide sequence encoding a chimeric antigen receptor (CAR) that binds BCMA, a disrupted TRAC gene, and a disrupted β2M gene. The nucleic acid comprising the CAR-coding sequence can be inserted into the disrupted TRAC gene. In some embodiments, the population of genetically engineered T cells may comprise ≥30% (e.g., about 35-70%) of anti-BCMA CAR+ T cells, ≤0.4% of TCR⁺ T cells, and/or ≤30% (e.g., about 15-30%) B2M⁺ T cells. In some examples, about 35-65% of the T cells in the T cell population are CAR⁺/TCR⁻/B2M⁻.

In some embodiments, the anti-BCMA CAR comprises (i) an ectodomain comprising an anti-BCMA single chain variable fragment (scFv); (ii) a CD8a transmembrane domain; and (iii) an endodomain comprising a co-stimulatory domain from 4-1BB and a CD3ζ signaling domain. For example, the anti-BCMA scFv may comprise a heavy chain variable domain (V_{H}) comprising SEQ ID NO: 42 and a light chain variable domain (V_{L}) comprising SEQ ID NO: 43. In some examples, the anti-BCMA scFv comprises the amino acid sequence of SEQ ID NO: 41. In some examples, the anti-BCMA CAR comprises the amino acid sequence of SEQ ID NO:40.

In some embodiments, the disrupted TRAC gene can be produced by a CRISPR/Cas9 gene editing system, which comprises a guide RNA comprising a spacer sequence of SEQ ID NO: 3 or SEQ ID NO:4. In some examples, the disrupted TRAC gene has a deletion of SEQ ID NO:10. Alternatively or in addition, the nucleotide sequence of SEQ ID NO: 30, which encodes an anti-BCMA CAR, is inserted into the TRAC gene, for example, substituting for the deleted fragment comprising SEQ ID NO:10.

In some embodiments, the disrupted β2M gene can be produced by a CRISPR/Cas9 gene editing system, which may comprise a guide RNA comprising a spacer sequence of SEQ ID NO:7 or SEQ ID NO:8. In some examples, the disrupted β2M gene may comprises at least one SEQ ID NOs: 21-26.

Further, the instant disclosure provides a composition comprising any of the population of genetically engineered T cells set forth herein and a cryopreservation solution, in which the population of genetically engineered T cells is suspected. In some embodiments, the cryopreservation solution may comprise about 2-10% dimethyl sulfoxide (DMSO), optionally about 5% DMSO, and is substantially free of serum. In some embodiments, the composition can be placed in a storage vial. In some examples, each storage vial contains about 25-85×10⁶ cells/ml.

In another aspect, provided herein is a method for treating multiple myeloma (MM) using any of the genetically engineered T cell populations disclosed herein or any of the compositions comprising such as also disclosed herein. In some embodiments, the method may comprise: (i) administering to a subject in need thereof an effective amount of one or more lymphodepleting chemotherapeutic agents; and (ii) administering to the subject an effective amount of any of the population of genetically engineered T cells as disclosed herein after step (i).

In some embodiments, the effective amount of the population of genetically engineered T cells given to a subject such as a human patient is sufficient to achieve one or more of the following: (a) decrease soft tissue plasmacytomas sizes (SPD) by at least 50% in the subject; (b) decrease serum M-protein levels by at least 25%, optionally by 50% in the subject; (c) decrease 24-hour urine M-protein levels by at least 50%, optionally by 90% in the subject; (d) decrease differences between involved and uninvolved free light chain (FLC) levels by at least 50% in the subject; (e) decrease plasma cell counts by at least 50% in the subject; (f) decrease kappa-to-lambda light chain ratios (κ/λ ratios) to 4:1 or lower in the subject, who has myeloma cells that produce kappa light chains; and (g) increase kappa-to-lambda light chain ratios (κ/λ ratios) to 1:2 or higher in the subject, who has myeloma cells that produce lambda light chains. In some examples, the effective amount of the population of genetically engineered T cells given to the subject is sufficient to decrease serum M-protein levels by at least 90% and 24-hour urine M-protein levels to less than 100 mg in the subject, and/or wherein the effective amount of the population of genetically engineered T cells is sufficient to decrease serum M-proteins, urine M-proteins, and soft tissue plasmacytomas to undetectable levels, and plasma cell counts to less than 5% of bone marrow (BM) aspirates in the subject.

In some examples, the effective amount of the population of genetically engineered T cells ranges from about 2.5×10⁷ to about 7.5×10⁸ CAR⁺ T cells (e.g., about 5.0×10⁷ to about 7.5×10⁸ CAR⁺ T cells). For example, the effective amount of the population of genetically engineered T cells in step (ii) ranges from about 5.0×10⁷ to about 1.5×10⁸ CAR⁺ T cells, about 1.5×10⁸ to about 4.5×10⁸ CAR⁺ T cells, about 4.5×10⁸ to about 6.0×10⁸ CAR⁺ T cells, or about 6.0×10⁸ to about 7.5×10⁸ CAR⁺ T cells.

In some examples, the effective amount of the population of genetically engineered T cells is about 2.5×10⁷ CAR⁺ T cells. In some examples, the effective amount of the population of genetically engineered T cells is about 5×10⁷ CAR⁺ T cells. In some examples, the effective amount of the population of genetically engineered T cells is about 1.5×10⁸ CAR⁺ T cells. In some examples, the effective amount of the population of genetically engineered T cells is about 4.5×10⁸ CAR⁺ T cells. In some examples, the effective amount of the population of genetically engineered T cells is about 6×10⁸ CAR⁺ T cells. In some examples, the effective amount of the population of genetically engineered T cells is about 7.5×10⁸ CAR⁺ T cells. Preferably, the effective amount of the population of genetically engineered T cells is at least 1.5×10⁸ CAR⁺ T cells, at least 4.5×10⁸ CAR⁺ T cells, or at least 6.0×10⁸ CAR⁺ T cells.

In some embodiments, step (i) of any of the methods disclosed herein comprises co-administering to the subject fludarabine at about 30 mg/m² and cyclophosphamide at about 300 mg/m² intravenously per day for three days. In some embodiments, step (i) of any of the methods disclosed herein comprises co-administering to the subject fludarabine at about 30 mg/m² and cyclophosphamide at about 500 mg/m² intravenously per day for three days. In some embodiments, step (ii) is performed 2-7 days after step (i).

In any of the methods disclosed herein, the human patient does not show one or more of the following features prior to step (i): (a) significant worsening of clinical status, (b) requirement for supplemental oxygen to maintain a saturation level of greater than about 91%, (c) uncontrolled cardiac arrhythmia, (d) hypotension requiring vasopressor support, (e) active infection, and (f) neurological toxicity that increases risk of immune effector cell-associated neurotoxicity syndrome (ICANS). Alternatively or in addition, the human patient does not show one or more of the following features prior to step (ii) and after step (i): (a) active uncontrolled infection, (b) worsening of clinical status compared to the clinical status prior to step (i), and (c) neurological toxicity that increases risk of immune effector cell-associated neurotoxicity syndrome (ICANS).

Any of the methods disclosed herein may further comprise (iii) monitoring the human patient for development of acute toxicity after step (ii). In some embodiments, acute toxicity comprises cytokine release syndrome (CRS), neurotoxicity, tumor lysis syndrome, hemophagocytic lymphohistiocytosis (HLH), cytopenias, GvHD, hypotention, renal insufficiency, viral encephalitis, neutropenia, thrombocytopenia, or a combination thereof. When toxicity is developed during the allogenic anti-BCMA CAR-T cell therapy, the subject is subject to toxicity management if development of toxicity is observed.

1) A subject suitable for the allogenic anti-BCMA CAR-T cell therapy as disclosed herein may be a human patient, who optionally is 18 years of age or older. The human patient may have one or more of the following features: (1) adequate organ function, (2) free of a prior allogeneic stem cell transplantation (SCT), (3) free of autologous SCT within 60 days prior to step (i), (4) free of plasma cell leukemia, non-secretory MM, Waldenstrom's macroglobulinemia, POEM syndrome, and/or amyloidosis with end organ involvement and damage, (5) free of prior gene therapy, anti-BCMA therapy, and non-palliative radiation therapy within 14 days prior to step (i), (6) free of central nervous system involvement by MM, (7) free of history or presence of clinically relevant CNS pathology, cerebrovascular ischemia and/or hemorrhage, dementia, a cerebellar disease, an autoimmune disease with CNS involvement, (8) free of unstable angina, arrhythmia, and/or myocardial infarction within 6 month prior to step (i), (9) free of uncontrolled infections, optionally wherein the infection is caused by HIV, HBV, or HCV, (10) free of previous or concurrent malignancy, provided that the malignancy is not basal cell or squamous cell skin carcinoma, adequately resected and in situ carcinoma of cervix, or a previous malignancy that was completely resected and has been in remission for ≥5 years, (11) free of live vaccine administration within 28 days prior to step (i), (12) free of systemic anti-tumor therapy within 14 days prior to step (i), and (13) free of primary immunodeficiency disorders or autoimmune disorders that require immunosuppressive therapy. Alternatively or in addition, the subject has measurable disease and/or Eastern Cooperative Oncology Group performance status 0 or 1.

In some embodiments, the subject may have relapsed and/or refractory MM. In some embodiments, the subject may have undergone at least two prior therapies for MM, which may comprise an immunomodulatory agent, a proteasome inhibitor, an anti-CD38 antibody, or a combination thereof. In some examples, the subject is double-refractory to prior therapies comprising an immunomodulatory agent and a proteasome inhibitor. In other examples, the subject is triple-refractory to prior therapies comprising an immunomodulatory agent, a proteasome inhibitor, and an anti-CD38 antibody. In other examples, the subject relapsed after an autologous stem cell transplant (SCT), which may occur within 12 months after the SCT. In yet other examples, the subject may be a patient who is triple-refractory to a proteasome inhibitor, an immunomodulatory agent, and an anti-CD38 antibody.

Further, the present disclosure features, in some aspects, a kit for use in treating multiple myeloma (e.g., refractory and/or relapsed MM). The kit comprises (a) a population of the genetically engineered anti-BCMA CAR-T cells disclosed herein or a composition comprising such as also disclosed herein, and (b) a vial, in which the population of genetically engineered anti-BCMA CAR-T cells or the composition is placed.

Also within the scope of the present disclosure is a composition for use in treating multiple myeloma (MM) such as refractory and/or relapsed MM, wherein the composition comprises any of the genetically engineered anti-BCMA CAR-T cells as disclosed herein, or use of the composition for manufacturing a medicament for use in treating multiple myeloma.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic illustration depicting an example allogeneic T cell comprising a disrupted TRAC gene and a disrupted β2M gene and expressing a chimeric antigen receptor (CAR) as illustrated. The T cell expresses an anti-BCMA CAR but does not express a functional TCR or MHC I complex.

FIG. 2 is a diagram depicting the percentage of TCR⁻, β2M⁻, anti-BCMA CAR⁺ and TCR⁻/β2M⁻/anti-BCMA CAR⁺ cells in a population of genetically engineered T cells (CTX120 cells), as measured by flow cytometry.

FIGS. 3A-3B include diagrams depicting the percentage of CD4⁺ (FIG. 3A) or CD8⁺ (FIG. 3B) T cells within a population of genetically engineered cells (CTX120 cells) or unedited cells as measured by flow cytometry.

FIG. 4 is a diagram depicting the volume of subcutaneous BCMA-expressing human MM tumors (MM.1S tumors) measured over time in immunocompromised mice that were untreated or treated with CTX120 cells on day 0. Circles depict the growth of primary tumors inoculated in the right flank in treated or untreated animals, with all untreated animals requiring euthanasia due to tumor burden and all treated animals rejecting primary tumors. Surviving treated animals were re-challenged with tumor cells on day 29 by inoculation with tumors cells in the left flank. Open triangles depict the growth of re-challenge tumors in treated animals, while closed triangles depict growth of tumors inoculated in the left flank of a new cohort of untreated animals.

FIG. 5 is a diagram depicting the volume of subcutaneous BCMA-expressing human MM tumors (RPMI-8226 tumors) measured over time in immunocompromised mice that were untreated or treated with CTX120 cells on day 1.

FIGS. 6A-6B include charts depicting production of interferon-gamma (IFNγ) (FIG. 6A) or interleukin-2 (IL-2) (FIG. 6B) by effector CTX120 cells following in vitro co-culture with tumor cells positive for surface expression of BCMA (MM.1S and JeKo-1) or negative for expression of BCMA (K562).

FIGS. 7A-7C include diagrams depicting the percentage of target cells characterized as dead/dying by flow cytometry following in vitro co-culture with unedited cells or edited CTX120 cells at different T cell to target cell ratios. The target cells were high BCMA-expressing MM.1S cells (FIG. 7A), low BCMA-expressing JeKo-1 cells (FIG. 7B), or BCMA-negative K562 cells (FIG. 7C).

FIGS. 8A-8B include charts depicting the production of IFNγ (FIG. 8A) or IL-2 (FIG. 8B) by effector CTX120 cells following in vitro co-culture with primary cells derived from human tissues, including B cells that contain BCMA-expressing cells, as compared to BCMA-expressing JeKo-1 cells as a positive control.

FIG. 9 is a diagram depicting the viability of an ex vivo culture of edited CTX120 cells over time as measured by cell counting when grown in complete media (serum+cytokines), media with serum (no cytokines), or media lacking serum and cytokines.

FIG. 10 is a diagram depicting survival of mice over time following exposure to a dosage of radiation and treatment with vehicle-only (no T cells), unedited T cells or edited CTX120 cells.

FIG. 11 is a chart depicting proliferation of unedited T cells or edited TRAC−/B2M− T cells following in vitro co-culture with peripheral blood mononuclear cells (PBMCs) derived from the same donor (autologous PBMCs) or a different donor (allogeneic PBMCs). As a positive control, T cells were stimulated with phytohaemagglutinin-L (PHA) to induce proliferation.

FIG. 12 is a diagram depicting the design of a clinical study to evaluate safety and efficacy of allogeneic CTX120 cells for treatment of human MM. Lymphodepleting chemotherapy is performed by co-administration of fludarabine at 30 mg/m² and cyclophosphamide 300 mg/m² IV daily for 3 days or by co-administration of fludarabine at 30 mg/m² and cyclophosphamide 500 mg/m² IV daily for 3 days. D: day; DLT: dose-limiting toxicity; IV: intravenously; M: month.

FIG. 13 is a schematic illustration depicting production of anti-BCMA CAR-T cells such as CTX120 cells.

### DETAILED DESCRIPTION OF THE INVENTION

B-cell maturation antigen (BCMA), also known as tumor necrosis factor receptor superfamily member 17 (TNFRSF17), is an antigenic determinant expressed by mature B cells. However, BCMA is differentially expressed in certain types of hematologic malignancies, wherein expression of BCMA is higher on malignant tumor cells than healthy cells. For example, BCMA is selectively expressed on the surface of multiple myeloma (MM) plasma cells and differentiated plasma cells, but not on memory B cells, naïve B cells, CD34+ hematopoietic stem cells, and other normal tissue cells (Cho, et al., (2018) Front Immuno 9:1821). Without being bound by theory, BCMA is thought to promote the proliferation and survival of MM cells, as well as promote an immunosuppressive bone marrow microenvironment that protects the MM cells from immune detection.

The present disclosure is based, in part, on the development of allogenic T cell therapy comprising genetically engineered T cells having disrupted endogenous TRAC and β2M genes and expressing an anti-BCMA CAR. Administration of the genetically engineered anti-BCMA CAR-T cells successfully eradicated human MM tumors that express BCMA as observed in animal models. Significantly, it has been observed that administration of the anti-BCMA CAR-T cells eliminated tumor burden and protected animals from re-challenge with tumors cells. Further, the genetically engineered anti-BCMA CAR-T cells, having disrupted endogenous TRAC and β2M genes did not induce graft versus host disease (GvHD) or host versus graft disease (HvGD) in animal models. Accordingly, the allogenic anti-BCMA CAR-T therapy disclosed herein are expected to be highly effective and safe in treating cancer such as MM in human patients.

### I. Genetically Engineered Anti-BCMA CAR-T Cells

In some aspects, the present disclosure provides a population of genetically engineered T cells expressing a CAR that specifically binds to BCMA (an anti-BCMA CAR or anti-BMCA CAR-T cells). In some embodiments, at least a portion of the genetically engineered T cells comprise: a nucleic acid encoding an anti-BCMA CAR; a disrupted gene associated with graft-versus-host disease (GvHD); and/or a disrupted gene associated with host-versus-graft (HvG) response. Methods of producing and using anti-BCMA CAR T cells are described in WO/2019/097305 and WO/2019/215500, the relevant disclosures of each of which are incorporated by reference herein for the purpose and subject matter referenced herein.

(i) Chimeric Antigen Receptor (CAR) Targeting BCMA (Anti-BCMA CAR)

A chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

The anti-BCMA CAR disclosed herein refers to a CAR capable of binding to a BCMA molecule, preferably a BCMA molecule expressed on cell surfaces. The human and murine amino acid and nucleic acid sequences of BCMA can be found in a public database (e.g., GenBank, UniProt, or Swiss-Prot). See, e.g., UniProt/Swiss-Prot Accession Nos. Q02223 (human BCMA) and 088472 (murine BCMA). In general, an anti-BCMA CAR is a fusion polypeptide comprising an extracellular domain (ectodomain) that recognizes BCMA (e.g., a single chain fragment (scFv) of an antibody or other antibody fragment) and an intracellular domain (endodomain) comprising a signaling domain of the T-cell receptor (TCR) complex (e.g., CD3ζ) and, in most cases, a co-stimulatory domain. (Enblad et al., Human Gene Therapy. 2015; 26(8):498-505). The anti-BCMA CAR disclosed herein may further comprise a hinge and transmembrane domain between the extracellular domain and the intracellular domain, as well as a signal peptide at the N-terminus for surface expression. Examples of signal peptides include MLLLVTSLLLCELPHPAFLLIP (SEQ ID NO: 54) and MALPVTALLLPLALLLHAARP (SEQ ID NO: 55). Other signal peptides may be used. In some examples, the anti-BCMA CAR may further comprise an epitope tag such as a GST tag or a FLAG tag.

(a) Antigen Binding Extracellular Domain

The antigen-binding extracellular domain is the region of a CAR polypeptide that is exposed to the extracellular fluid when the CAR is expressed on cell surface. In some instances, a signal peptide may be located at the N-terminus to facilitate cell surface expression. In some embodiments, the antigen binding domain can be a single-chain variable fragment (scFv), which may include an antibody heavy chain variable region (V_{H}) and an antibody light chain variable region (V_{L}) (in either orientation). In some instances, the V_{H} and V_{L} fragment may be linked via a peptide linker. The linker, in some embodiments, includes hydrophilic residues with stretches of glycine and serine for flexibility as well as stretches of glutamate and lysine for added solubility. The linker peptide may be about 10 to about 25 amino acids. In specific examples, the linker peptide comprises a sequence set forth in SEQ ID NO: 53 (Table 5). The scFv fragment retains the antigen-binding specificity of the parent antibody, from which the scFv fragment is derived. In some embodiments, the scFv may comprise humanized V_{H} and/or V_{L} domains. In other embodiments, the V_{H} and/or V_{L} domains of the scFv are fully human.

The antigen-binding extracellular domain of the anti-BCMA CAR disclosed herein is capable of binding to a BCMA molecule, preferably a BCMA molecule expressed on cell surface. The antigen-binding extracellular domain can be an antibody specific to BCMA or an antigen-binding fragment thereof. In some embodiments, the antigen-binding extracellular domain (the BCMA-binding domain) comprises a single-chain variable fragment (scFv), which may be derived from a suitable antibody, for example, a murine antibody, a rat antibody, a rabbit antibody, a human antibody, or a chimeric antibody. In some instances, the scFv is derived from a human anti-BCMA antibody. In other instances, the anti-BCMA scFv is humanized (e.g., fully humanized). For example, the anti-BCMA scFv is humanized and comprises one or more residues from complementarity determining regions (CDRs) of a non-human species, e.g., from mouse, rat, or rabbit.

In some embodiments, the anti-BCMA scFv comprises an antibody heavy chain variable region (V_{H}) and an antibody light chain variable region (V_{L}) (in either orientation), which comprise the same heavy chain complementary determining regions (CDRs) as the V_{H} of SEQ ID NO:42 and the same light chain CDRs as the V_{L} of SEQ ID NO:43. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). For example, the anti-BCMA scFv may comprise the heavy chain and light chain CDR1s, CDR2s, and CDR3s provided in Table 5 below, following the Kabat approach. Alternative, the anti-BCMA scFv may comprise the heavy chain and light chain CDR1s, CDR2s, and CDR3s provided in Table 5 below, following the Chothia approach.

In other examples, the anti-BCMA scFv used in any of the anti-BCMA CAR constructs disclosed herein may be a functional variant of an anti-BCMA scFv comprising the amino acid sequence of SEQ ID NO:41 (exemplary anti-BCMA scFv). Such functional variants are substantially similar to the exemplary antibody, both structurally and functionally. A functional variant comprises substantially the same V_{H} and V_{L} CDRs as the exemplary anti-BCMA antibody. For example, it may comprise only up to 8 (e.g., 8, 7, 6, 5, 4, 3, 2, or 1) amino acid residue variations in the total CDR regions of the exemplary anti-BCMA scFv and binds the same epitope of BCMA with substantially similar affinity (e.g., having a K_{D} value in the same order).

For example, an anti-BCMA scFv disclosed herein may comprises: a) a V_{L} CDR1 comprising SEQ ID NO: 44, or a sequence having 1 to 3 amino acid substitutions relative to SEQ ID NO: 44; b) a V_{L} CDR2 comprising SEQ ID NO: 45, or a sequence having 1 amino acid substitution relative to SEQ ID NO: 45; c) a V_{L} CDR3 comprising SEQ ID NO: 46, or a sequence having 1 to 2 amino acid substitutions relative to SEQ ID NO: 46; and/or d) a V_{H} CDR1 comprising SEQ ID NO: 47, or a sequence having 1 amino acid substitution relative to SEQ ID NO: 47; e) a V_{H} CDR2 comprising SEQ ID NO: 48, or a sequence having 1 to 3 amino acid substitutions relative to SEQ ID NO: 48; f) a V_{H} CDR3 comprising SEQ ID NO: 49, or a sequence having 1 to 2 amino acid substitutions relative to SEQ ID NO: 49, or any combination thereof. See Table 5. In some examples, the anti-BCMA scFv comprises: a V_{L} CDR1 comprising SEQ ID NO: 44, a V_{L} CDR2 comprising SEQ ID NO: 45, a V_{L} CDR3 comprising SEQ ID NO: 46, a V_{H} CDR1 comprising SEQ ID NO: 47, a V_{H} CDR2 comprising SEQ ID NO: 48, and a V_{H} CDR3 comprising SEQ ID NO: 49.

In other examples, the anti-BCMA scFv may comprise: a) a V_{L} CDR1 comprising SEQ ID NO: 44, or a sequence having 1 to 3 amino acid substitutions relative to SEQ ID NO: 44; b) a V_{L} CDR2 comprising SEQ ID NO: 45, or a sequence having 1 amino acid substitution relative to SEQ ID NO: 45; c) a V_{L} CDR3 comprising SEQ ID NO: 46, or a sequence having 1 to 2 amino acid substitutions relative to SEQ ID NO: 46; and/or d) a V_{H} CDR1 comprising SEQ ID NO: 50, or a sequence having 1 amino acid substitution relative to SEQ ID NO: 50; e) a V_{H} CDR2 comprising SEQ ID NO: 51, or a sequence having 1 amino acid substitution relative to SEQ ID NO: 51; f) a V_{H} CDR3 comprising SEQ ID NO: 52, or a sequence having 1 to 2 amino acid substitutions relative to SEQ ID NO: 52, or any combination thereof (Table 5). In some embodiments, the anti-BCMA scFv comprises: a V_{L} CDR1 comprising SEQ ID NO: 44, a V_{L} CDR2 comprising SEQ ID NO: 45, a V_{L} CDR3 comprising SEQ ID NO: 46, a V_{H} CDR1 comprising SEQ ID NO: 50, a V_{H} CDR2 comprising SEQ ID NO: 51, and a V_{H} CDR3 comprising SEQ ID NO: 52.

In some instances, the amino acid residue variations or substitution in one or more of the CDRs disclosed herein can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-BCMA scFv disclosed herein may comprise heavy chain CDRs that are at least 80% (e.g., 85%, 90%, 95%, or 98%) sequence identity, individually or collectively, as compared with the V_{H} CDRs of the exemplary anti-BCMA scFv of SEQ ID NO:41. Alternatively or in addition, the anti-BCMA scFv may comprise light chain CDRs that are at least 80% (e.g., 85%, 90%, 95%, or 98%) sequence identity, individually or collectively, as compared with the V_{L} CDRs as the exemplary anti-BCMA scFv. As used herein, “individually” means that one CDR of an antibody shares the indicated sequence identity relative to the corresponding CDR of the exemplary antibody. “Collectively” means that three V_{H} or V_{L} CDRs of an antibody in combination share the indicated sequence identity relative the corresponding three V_{H} or V_{L} CDRs of the exemplary antibody in combination.

In some examples, the anti-BCMA scFv may comprise a V_{H} domain that comprises an amino acid sequence at least 80%, at least 85%, at least 90%, at least 95%, at least 96%, at least 97%, at least 98%, at least 99%, or 100% identical to a sequence set forth in SEQ ID NO: 42 (Table 5). Alternatively or in addition, the anti-BCMA scFv may comprise a V_{L} domain that comprises an amino acid sequence at least 80%, at least 85%, at least 90%, at least 95%, at least 96%, at least 97%, at least 98%, at least 99%, or 100% identical to a sequence set forth in SEQ ID NO: 43 (Table 5). In some examples, the linker peptide connects the N-terminus of the anti-BCMA V_{H} with the C-terminus of the anti-BCMA V_{L}. Alternatively, the linker peptide connects the C-terminus of the anti-BCMA V_{H} with the N-terminus of the anti-BCMA V_{L}.

In some examples, the anti-BCMA scFv may comprise an amino acid sequence at least 80%, at least 85%, at least 90%, at least 95%, at least 96%, at least 97%, at least 98%, at least 99%, or 100% identical to a sequence set forth in SEQ ID NO: 41.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

(b) Transmembrane Domain

The CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the CAR containing such.

In some embodiments, the transmembrane domain of a CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain Other transmembrane domains may be used as provided herein. In some embodiments, the transmembrane domain is a CD8a transmembrane domain containing the sequence of FVPVFLPAKPTTTPAPRPPTPAPTIASQPLSLRPEACRPAAGG AVHTRGLDFACDIYIWAPLAGTCGVLLLSLVITLYCNHRNR (SEQ ID NO: 60) or IYIWAPLAGTCGVLLLSLVITLY (SEQ ID NO: 56). In some embodiments, the CD8a transmembrane domain may comprise an amino acid sequence at least 80%, at least 85%, at least 90%, at least 95%, at least 96%, at least 97%, at least 98%, at least 99%, or 100% identical to a sequence set forth in SEQ ID NO: 56. Other transmembrane domains may be used.

(c) Hinge Domain

In some embodiments, the anti-BCMA CAR further comprises a hinge domain, which may be located between the extracellular domain (comprising the antigen binding domain) and the transmembrane domain of the CAR, or between the cytoplasmic domain and the transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a CAR. In some embodiments, the hinge domain may be a CD8 hinge domain Other hinge domains may be used.

In some embodiments, the hinge domain comprises about 5 to about 300 amino acids, e.g., about 5 to about 250, about 10 to about 250, about 10 to about 200, about 15 to about 200, about 15 to about 150, about 20 to about 150, about 20 to about 100, about 25 to about 100, about 25 to about 75, or about 30 to about 750 amino acids. In some embodiments, the anti-BCMA hinge domain comprises a CD8a hinge domain and, optionally, an extension comprising an additional 1-10 amino acids (e.g., 4 amino acids) at the N-terminus of the hinge domain. In some examples, the extension comprises amino acid sequence SAAA.

(d) Intracellular Signaling Domains

Any of the CAR constructs contain one or more intracellular signaling domains (e.g., CD3ζ, and optionally one or more co-stimulatory domains), which are the functional end of the receptor. Following antigen recognition, receptors cluster and a signal is transmitted to the cell. CD3ζ is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3ζ provides a primary T cell activation signal but not a fully competent activation signal, which requires a co-stimulatory signaling. In some embodiments, the CD3ζ signaling domain comprises an amino acid sequence at least 80%, at least 85%, at least 90%, at least 95%, at least 96%, at least 97%, at least 98%, or at least 99%, or 100% identical to a sequence set forth in SEQ ID NO: 59 (Table 5).

In some embodiments, the CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3ζ. In some examples, the CAR disclosed herein comprises a CD28 co-stimulatory molecule. In other examples, the CAR disclosed herein comprises a 4-1BB co-stimulatory molecule. In some embodiments, a CAR includes a CD3ζ signaling domain and a CD28 co-stimulatory domain. In other embodiments, a CAR includes a CD3ζsignaling domain and 4-1BB co-stimulatory domain. In still other embodiments, a CAR includes a CD3 signaling domain, a CD28 co-stimulatory domain, and a 4-1BB co-stimulatory domain.

In some examples, the anti-BCMA CAR comprises a 4-1BB co-stimulatory domain. The 4-1BB co-stimulatory domain may comprise an amino acid sequence at least 80%, at least 85%, at least 90%, at least 95%, at least 96%, at least 97%, at least 98%, at least 99%, or 100% identical to a sequence set forth in SEQ ID NO: 57 (Table 5).

In some examples, the anti-BCMA CAR comprises a CD28 co-stimulatory domain. The CD28 co-stimulatory domain may comprise an amino acid sequence at least 80%, at least 85%, at least 90%, at least 95%, at least 96%, at least 97%, at least 98%, at least 99%, or 100% identical to a sequence set forth in SEQ ID NO: 58 (Table 5).

(e) Exemplary Anti-BCMA CAR

In some examples, the anti-BCMA CAR disclosed herein comprises, from the N-terminus to the C-terminus, a CD8 signaling peptide (e.g., SEQ ID NO:55), an anti-BCMA scFv (e.g., SEQ ID NO:41), a CD8a transmembrane domain (e.g., SEQ ID NO:56), a 4-1BB co-stimulatory domain (e.g., SEQ ID NO: 57), and a CD3z signaling domain (e.g., SEQ ID NO:59). Such an anti-BCMA CAR may comprise an amino acid sequence at least 80%, at least 85%, at least 90%, at least 95%, at least 96%, at least 97%, at least 98%, at least 99%, or 100% identical to a sequence set forth in SEQ ID NO: 40 (Table 5). The anti-BCMA CAR may be encoded by a nucleic acid comprising a sequence at least 80%, at least 85%, at least 90%, at least 95%, at least 96%, at least 97%, at least 98%, at least 99%, or 100% identical to a sequence set forth in SEQ ID NO: 33 (Table 4).

In specific examples, the anti-BCMA CAR is CTX-166b, which comprises the amino acid sequence of SEQ ID NO: 40 (Table 5).

It should be understood that methods described herein encompasses more than one suitable CAR that can be used to produce genetically engineered T cells expressing the CAR, for example, those known in the art or disclosed herein. Examples can be found in WO2019/097305 and WO/2019/215500, the relevant disclosures of each of which are incorporated by reference for the purpose and subject matter referenced herein.

Expression of any of the anti-BCMA CAR (e.g., CTX-166b) can be driven by an endogenous promoter at the integration site. Alternatively, expression of the anti-BCMA CAR can be driven by an exogenous promoter. For example, an exogenous EF1α promoter (e.g., comprising the nucleotide sequence of SEQ ID NO: 38; see Table 4) can be located directly upstream of the nucleic acid sequence encoding the anti-BCMA CAR. In some embodiments, the anti-BCMA CAR expression cassette may further comprise an exogenous enhancer, an insulator, an internal ribosome entry site, a sequence encoding 2A peptides, a 3′ polyadenylation (poly A) signal, or a combination thereof. In specific examples, the 3′ poly A signal comprises a nucleotide sequence set forth in SEQ ID NO: 39 (Table 4).

(ii) Genetic Modification of TRAC and B2M Endogenous Genes

The anti-BCMA CAR-T cells may be further modified genetically to disrupt an endogenous gene associated with GvHD (e.g., a gene encoding a component of TCR such as a TRAC gene), an endogenous gene associated with HvGD (e.g., a β2M gene).

It should be understood that gene disruption encompasses gene modification through gene editing (e.g., using CRISPR/Cas gene editing to insert or delete one or more nucleotides). As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein.

Disrupted TRAC Gene

GvHD is commonly seen in the setting of allogeneic stem cell transplantation (SCT). Immunocompetent donor T cells (the graft) recognize the recipient (the host) as foreign and become activated to attack the recipient to eliminate “foreign antigen-bearing” host cells. Clinically, GvHD is divided into acute, chronic, and overlap syndrome based upon clinical manifestations and the time of incidence relative to administration of allogeneic donor cells. Symptoms of acute GvHD (aGvHD) can include maculopapular rash; hyperbilirubinemia with jaundice due to damage to the small bile ducts, leading to cholestasis; nausea, vomiting, and anorexia; and watery or bloody diarrhea and cramping abdominal pain (Zeiser, R. et al. (2017) N Engl J Med 377:2167-79). The severity of aGvHD is based upon clinical manifestations and is readily evaluated by one skilled in the art using widely accepted grading parameters as defined, for example, in Table 11.

In some embodiments, the anti-BCMA CAR-T cells have a disrupted endogenous gene associated with GvHD, for example, an endogenous TRAC gene, to reduce the risk or eliminate GvHD when the anti-BCMA CAR-T cells are administered to a recipient. In some embodiments, the disrupted TRAC gene may comprise a deletion, a nucleotide residue substation, an insertion, or a combination thereof. Structure of a disrupted TRAC gene would depend on the gene editing method used to disrupt the endogenous TRAC gene. For example, the TRAC gene may be disrupted by the CRISPR/Cas9 system using a suitable guide RNA (e.g., those disclosed herein. See Table 1 and Example 1 below). Such a gene editing approach may create deletions, insertions, and/or nucleotide substitutions nearby the gene locus targeted by the guide RNA (gRNA).

In some embodiments, the genetically engineered anti-BCMA CAR-T cell comprises a disrupted TRAC gene, which comprises an insertion and/or a deletion. In some examples, the insertion and/or deletion is within Exon 1. In specific examples, the disrupted TRAC gene has a deletion of a fragment comprising SEQ ID NO: 10. In some examples, the fragment comprising SEQ ID NO:10 may be replaced with a nucleic acid encoding the anti-BCMA CAR, for example, SEQ ID NO: 30. Alternatively or in addition, the disrupted TRAC gene may comprise an insertion of a nucleic acid, which comprises a nucleotide sequence encoding any of the anti-BCMA CAR. In some examples, the anti-BCMA CAR-encoding sequence may be flanked by a left homology arm and a right homology arm, which comprise homologous sequences flanking the region targeted by the gene editing method for use in disrupting the TRAC gene in the T cells. In some instances, the left homology arm and the right homology arm comprise sequences homologous to a 5′ end and a 3′ end site nearby the region of SEQ ID NO:10, respectfully, such that via homologous recombination, the nucleic acid encoding an anti-BCMA CAR is inserted into the disrupted TRAC locus. In specific examples, an exogenous nucleic acid comprising the nucleotide sequence of SEQ ID NO: 33 (encoding an anti-BCMA CAR comprising the amino acid sequence of SEQ ID NO:40) can be inserted into the TRAC gene, for example, inserted at or nearby the region of SEQ ID NO:10. The exogenous nucleic acid may further comprise a promoter in operative linkage to the coding sequence of the anti-BCMA CAR to drive expression of the anti-BCMA CAR in the genetically engineered T cells as disclosed herein. In some examples, the promoter can be an EF-1a promoter, which may comprise the nucleotide sequence of SEQ ID NO: 38. Alternatively or in addition, the exogenous nucleic acid may further comprise a poly A sequence downstream of the anti-BCMA CAR coding sequence.

Disrupted B2M Gene

HvGD refers to the immune rejection of donor cells, for example, tumor-targeting CAR T cells, by the recipient's immune system. Risk of tumor relapse with tumor-targeting CAR T cell therapy is thought to be due, in part, to limited persistence of CAR T cells in a subject following administration (Maude, S., et al. (2014) N Engl J Med. 371:1507-17; Turtle, C. et al., (2016) J Clin Invest. 126:2123-38). Elimination of allogeneic antigens from CAR T cells prior to transplantation can eliminate or reduce the risk of host rejections (e.g., a HvG response), thereby increasing persistence following administration.

In some embodiments, the genetically engineered anti-BCMA CAR-T cells may comprise a genetic disruption in a gene associated with HvGD, either alone or in combination with disruption of a gene associated with GvHD (e.g., TRAC gene disclosed herein). In some embodiments, the gene associated with HvGD encodes a component of major histocompatibility (MHC) class I molecules, for example, the β2M gene. Disruption of the gene associated with HvGD, e.g., disruption of the β2M gene, minimizes the risk of HvGD. Alternatively or in addition, the disruption of the β2M gene improves persistence of the CAR T cells.

In some embodiments, the genetically engineered anti-BCMA CAR-T cells comprise a disrupted β2M gene, either alone or in combination with a disrupted TRAC gene, comprises a genetic modification, which can be a deletion, an insertion, a nucleotide residue substitution, or a combination thereof. Structure of a disrupted β2M gene would depend on the gene editing method used to disrupt the endogenous β2M gene. For example, the β2M gene may be disrupted by the CRISPR/Cas9 system using a suitable guide RNA (e.g., those disclosed herein. See Table 1 and Example 1 below). Such a gene editing approach may create deletions, insertions, and/or nucleotide substitutions nearby the gene locus targeted by the guide RNA (gRNA).

In some examples, the disrupted β2M gene comprises a deletion, an insertion, a substitution, or a combination thereof in SEQ ID NO: 12 (Table 1). In examples, the disrupted β2M gene comprises at least one nucleotide sequence of any one of SEQ ID NO: 21-26 (Table 3).

(iii) Population of Anti-BCMA CAR-T Cells

The present disclosure also provides a population of genetically engineered anti-BCMA CAR-T cells disclosed herein, which express an anti-BCMA CAR and have a disrupted endogenous TRAC gene, an endogenous β2M gene, or both. In some embodiments, the population of the genetically engineered anti-BCMA CAR-T cells is heterogeneous, i.e., comprising genetically engineered T cells having different or different combination of the genetic modifications as disclosed herein (i.e., expression of anti-BCMA CAR, disrupted endogenous TRAC gene, and disrupted endogenous β2M gene). For example, the population of genetically engineered T cells may comprise a first group of T cells expressing the anti-BCMA CAR as disclosed herein and having a disrupted TRAC gene and a second group of T cells expressing the anti-BCMA CAR and a disrupted β2M gene. The first group and second group of the T cells may overlap. In some examples, a portion of the T cell population disclosed herein comprises all of the three genetic modifications, including expression of an anti-BCMA CAR, disrupted TRAC gene, and disrupted β2M gene.

In some embodiments, a portion of the population of genetically engineered T cells express an anti-BCMA CAR and comprise a disrupted TRAC gene, which may comprise an insertion, a deletion, a substitution, or a combination thereof. In some embodiments, the disruption of the TRAC gene eliminates or decreases expression of the TCR in the genetically engineered T cells. In some examples, 50% or less of the T cells express a TCR (TCR⁺), for example, 45% or less, 40% or less, 30% or less, 25% or less, 20% or less, 15% or less, 10% or less, 9% or less, 8% or less, 7% or less, 6% or less, 5% or less, 4% or less, 3% or less, 2% or less, 1% or less, 0.9% or less, 0.8% or less, 0.7% or less, 0.6% or less, 0.5% or less, 0.4% or less, 0.3% or less, 0.2% or less, or 0.1% or less. In some examples, 0.05%-50% of the genetically engineered T cells express a TCR, for example, 10%-50%, 20%-50%, 30%-50%, 40%-50%, 0.05%-40%, 10%-40%, 20%-40%, 30%-40%, 0.05%-30%, 10%-30%, 20%-30%, 0.05%-20%, 10%-20%, or 0.05%-10% of the genetically engineered T cells express a TCR. In some examples, 0.4% or less of the genetically engineered T cells express a TCR.

In some embodiments, the population of genetically engineered T cells elicits no clinical manifestations of GVHD response in a subject. For example, the genetically engineered T cells elicits no clinical manifestations of aGvHD (e.g., steroid-refractory aGvHD) in the subject. In some examples, the genetically engineered T cells elicits no clinically significant (e.g., grade 2-4) aGvHD in the subject. In some examples, the genetically engineered T cells elicits only mild aGvHD response (e.g., below clinical grade 2, 1, or 0) in the subject. In some examples, the genetically engineered T cells elicit clinically significant (e.g., grade 2-4) aGvHD (e.g., steroid-refractory aGvHD) in less than 18% of the subjects, e.g., less than 16%, less than 14%, less than 12%, less than 10%, less than 8%, less than 6%, less than 5%, less than 4%, less than 3%, less than 2%, or less than 1%.

In some embodiments, risk of GvHD (e.g., clinically significant aGvHD) elicited by the population of genetically engineered T cells as disclosed herein are reduced compared to a T cell population where at least 50% of the T cells express a TCR, e.g., at least 60%, at least 70%, at least 80%, at least 90%, or at least 95%. In some examples, the reduction in clinically significant aGvHD (e.g., grade 2-4) is at least 20%, at least 30%, at least 40%, at least 50%, at least 60%, at least 70%, at least 80%, at least 90%, or 100%.

In some embodiments, symptoms of aGvHD is observed for up to 36 days after administration of the population of genetically engineered T cells disclosed herein, e.g., up to 21 days, up to 24 days, up to 28 days, up to 30 days, or up to 35 days. In some examples, symptoms of aGvHD is observed for about 20 to about 50 days, about 25 to about 70 days, or about 28 to about 100 days after administration of the T cell population.

Alternatively or in addition, a portion of the genetically engineered T cells express an anti-BCMA CAR and comprise a disrupted β2M gene, which may comprise an insertion, a deletion, a substitution, or a combination thereof. In some embodiments, the disruption of the β2M gene eliminates or decreases expression of β2 microglobulin, leading to a loss of function of the MHC I complex. In some examples, 50% or less of the genetically engineered T cell population express β2 microglobulin, e.g., 45% or less, 40% or less, 35% or less, 30% or less, 25% or less, 20% or less, 15% or less, 10% or less, or 5% or less. In some examples, about 5% to about 50% of the genetically engineered T cells in the T cell population express 132 microglobulin, e.g., about 10%-50%, 10%-45%, 15%-45%, 15%-40%, 20%-40%, 20%-35%, or 25%-35%. In some examples, 30% or less of the genetically engineered T cells express β2 microglobulin.

In some embodiments, the genetic disruption of the gene associated with HvG (e.g., the β2M gene) eliminates or reduces the risk of HvGD response. Alternatively or in addition, the genetic disruption of the gene associated with HvGD (e.g., the β2M gene) increases the persistence of the allogeneic T cells in the subject. In some examples, a subject receiving the genetically engineered T cell population disclosed herein has no clinical manifestations of HvGD response. In some examples, the genetically engineered T cells are detectable in a tissue (e.g., in peripheral blood) of the subject at least 1 day after administration, e.g., at least 2, 4, 5, 7, 10, 14, 15, 20, 21, 25, 28, 30, or 35 days. The tissue may be obtained from peripheral blood, cerebrospinal fluid, tumor, skin, bone, bone marrow, breast, kidney, liver, lung, lymph node, spleen, gastrointestinal tract, tonsils, thymus, prostate, or a combination thereof.

Detectable is defined in terms of the limit of detection of a method of analysis. Persistence is the duration of time after administration where a detectable quantity of allogeneic T cells is measured. Methods for detecting or quantity T cells in a tissue of interest are known to those of skill in the art. Such methods include, but are not limited to, reverse transcription polymerase chain reaction (RT-PCR), competitive RT-PCR, real-time RT-PCR, RNase protection assay (RPA), quantitative immunofluorescence (QIF), flow cytometry, northern blotting, nucleic acid microarray using DNA, western blotting, enzyme-linked immunosorbent assay (ELISA), radioimmunoassay (RIA), tissue immunostaining, immunoprecipitation assay, complement fixation assay, fluorescence-activated cell sorting (FACS), mass spectrometry, magnetic bead-antibody immunoprecipitation, or protein chip.

In specific examples, the population of genetically engineered anti-BCMA CAR-T cells are CTX120 cells (see also Example 1 below), which are produced using CRISPR technology to disrupt targeted genes (TRAC and β2M), and adeno-associated virus (AAV) transduction to deliver the CAR construct of SEQ ID NO:40 CRISPR-Cas9-mediated gene editing involves two guide RNAs (sgRNAs): TA-1 sgRNA (SEQ ID NO: 1), which targets the TRAC locus, and B2M-1 sgRNA (SEQ ID NO: 5), which targets the β2M locus. The anti-BCMA CAR of the CTX120 cells is composed of an anti-BCMA single-chain antibody fragment (scFv) specific for BCMA, followed by a CD8 hinge and transmembrane domain that is fused to an intracellular co-signaling domain of 4-1BB and a CD3 signaling domain. The anti-BCMA scFv comprises the amino acid sequence of SEQ ID NO:41 and the anti-BCMA CAR comprises the amino acid sequence of SEQ ID NO: 40. Sequences of the other components in the anti-BCMA CAR are provided in Tables 4 and 5 below.

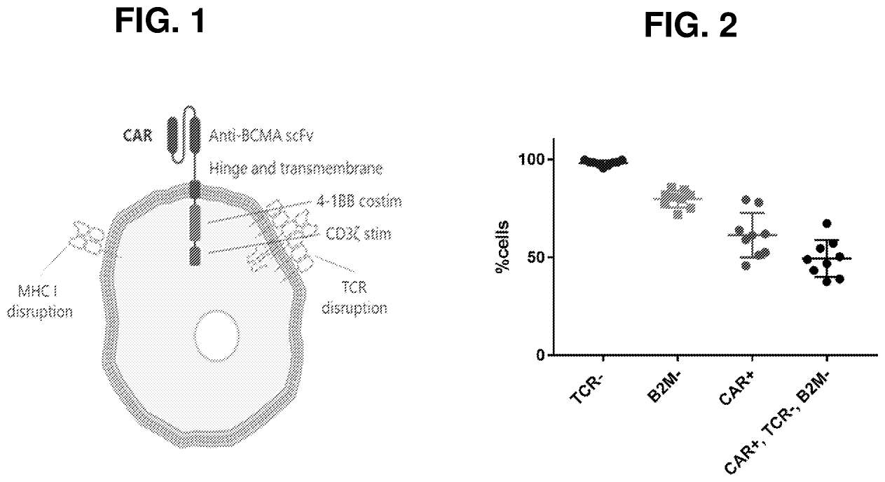

At least a portion of the CTX120 cells comprises anti-BCMA CAR-expressing T cells with a disrupted TRAC gene, in which the fragment of SEQ ID NO:10 is deleted. An exogenous nucleic acid configured for expressing the anti-BCMA CAR can be inserted into the TRAC gene. The exogenous nucleic acid comprises a promoter sequence (e.g., EF-1a promoter, which may comprise the nucleotide sequence of SEQ ID NO: 38), a nucleotide sequence coding for an anti-BCMA CAR (e.g., SEQ ID NO: 33, coding for the anti-BCMA CAR comprising the amino acid sequence of SEQ ID NO:40), and a poly A sequence (e.g., SEQ ID NO: 39) downstream of the coding sequence. The promoter sequence is in operable linkage to the coding sequence such that it drives expression of the anti-BCMA CAR in the CTX120 cells. At least a portion of the CTX120 cells comprise, collectively, a population of disrupted β2M genes, which may comprise one or more of nucleotide sequence of SEQ ID Nos: 21-26. See also FIG. 1 and Example 1 below.

Further, at least 30% of the T cells in the CTX120 cell population express the anti-BCMA CAR (CAR⁺ cells). In some examples, about 40% to about 80% (e.g., about 40%-75%, about 45%-75%, about 50%-70%, or about 50%-60%) of the T cells in the CTX120 cell population are CARP. In addition, less than 35% (e.g., ≤30%) at of the T cells in the CTX120 cell population express a detectable level of β2M surface protein. For example, about 70% to about 85% of the T cells in the CTX120 cell population do not express a detectable level of β2M surface protein. Moreover, less than about 1% (e.g., less than about 0.8%, less than 0.5%, or less than 4%) of the T cells in the CTX120 cell population express functional TCR.

At least a portion of the CTX120 T cells (e.g., at least 35%) are triple-modified CAR T cells, which refer to a genetically engineered T cell expressing the anti-BCMA CAR and having disrupted endogenous TRAC gene and endogenous β2M gene, e.g., produced by the CRISPR/Cas9 approach disclosed above and AAV-mediated delivery of the CAR construct. In some examples, about 35% to about 70% (e.g., about 40% to about 70% or about 50% to about 65%) of the T cells in the CTX120 cell population are triple-modified CAR T cells.

(iv) Pharmaceutical Compositions

In some aspects, the present disclosure provides pharmaceutical compositions comprising any of the genetically engineered anti-BCMA CAR T cells as disclosed herein, for example, CTX120 cells, and a pharmaceutically acceptable carrier. Such pharmaceutical compositions can be used in cancer treatment in human patients, which is also disclosed herein.

As used herein, the term “pharmaceutically acceptable” refers to those compounds, materials, compositions, and/or dosage forms which are, within the scope of sound medical judgment, suitable for use in contact with the tissues, organs, and/or bodily fluids of the subject without excessive toxicity, irritation, allergic response, or other problems or complications commensurate with a reasonable benefit/risk ratio. As used herein, the term “pharmaceutically acceptable carrier” refers to solvents, dispersion media, coatings, antibacterial agents, antifungal agents, isotonic and absorption delaying agents, or the like that are physiologically compatible. The compositions can include a pharmaceutically acceptable salt, e.g., an acid addition salt or a base addition salt. See, e.g., Berge et al., (1977) J Pharm Sci 66:1-19.

In some embodiments, the pharmaceutical composition further comprises a pharmaceutically acceptable salt. Non-limiting examples of pharmaceutically acceptable salts include acid addition salts (formed from a free amino group of a polypeptide with an inorganic acid (e.g., hydrochloric or phosphoric acids), or an organic acid such as acetic, tartaric, mandelic, or the like). In some embodiments, the salt formed with the free carboxyl groups is derived from an inorganic base (e.g., sodium, potassium, ammonium, calcium or ferric hydroxides), or an organic base such as isopropylamine, trimethylamine, 2-ethylamino ethanol, histidine, procaine, or the like).

In some embodiments, the pharmaceutical composition disclosed herein comprises a population of the genetically engineered anti-BCMA CAR-T cells (e.g., CTX120 cells) suspended in a cryopreservation solution (e.g., CryoStor® C55). In some instances, the cryopreservation solution may contain about 2-10% dimethyl sulfoxide (DMSO). For example, the cryopreservation solution may contain about 2%, about 3%, about 4%, about 5%, about 6%, about 7%, about 8%, about 9%, or about 10% DMSO. In specific examples, the cryopreservation solution may contain about 5% DMSO.

In addition to DMSO, a cryopreservation solution for use in the present disclosure may also comprise adenosine, dextrose, dextran-40, lactobionic acid, sucrose, mannitol, a buffer agent such as N-)2-hydroxethyl) piperazine-N′-(2-ethanesulfonic acid) (HEPES), one or more salts (e.g., calcium chloride, magnesium chloride, potassium chloride, potassium bicarbonate, potassium phosphate, etc.), one or more base (e.g., sodium hydroxide, potassium hydroxide, etc.), or a combination thereof. Components of a cryopreservation solution may be dissolved in sterile water (injection quality). Any of the cryopreservation solution may be substantially free of serum (undetectable by routine methods).

In some instances, a pharmaceutical composition comprising a population of genetically engineered anti-BCMA CAR-T cells such as the CTX120 cells suspended in a cryopreservation solution (e.g., comprising about 5% DMSO and optionally substantially free of serum) may be placed in storage vials. In some examples, each storage vial may contain about 25-85×10⁶ cells/ml of the T cells (e.g., CTX120). In some examples, each storage vial may contain about 50×10⁶ cells/ml. Among the cells in a storage vial, ≥30% are CAR⁺ T cells, ≤0.4% are TCR⁺ T cells, and ≤30% are B2M⁺ T cells.

Any of the pharmaceutical compositions disclosed herein, comprising a population of genetically engineered anti-BCMA CAR T cells as also disclosed herein (e.g., CTX120 cells), which optionally may be suspended in a cryopreservation solution (e.g., comprising about 5% DMSO and optionally substantially free of serum), may be stored in an environment that does not substantially affect viability and bioactivity of the T cells for future use, e.g., under conditions commonly applied for storage of cells and tissues. In some examples, the pharmaceutical composition may be stored in the vapor phase of liquid nitrogen at ≤−135° C. No significant changes were observed with respect to appearance, cell count, viability, % CAR⁺ T cells, % TCR⁺ T cells, and % B2M⁺ T cells after the cells have been stored under such conditions for a period of time.

### II. Preparation of Genetically Engineered Anti-BCMA CAR-T Cells

Any suitable gene editing methods known in the art can be used for making the genetically engineered anti-BCMA CAR T cells disclosed herein, for example, nuclease-dependent targeted editing using zinc-finger nucleases (ZFNs), transcription activator-like effector nucleases (TALENs), or RNA-guided CRISPR-Cas9 nucleases (CRISPR/Cas9; Clustered Regular Interspaced Short Palindromic Repeats Associated 9).

(a) Sources of T Cells

In some embodiments, primary T cells isolated from one or more donors may be used for making the genetically engineered anti-BCMA CAR-T cells. For example, primary T cells may be isolated from a suitable tissue of one or more healthy human donors, e.g., peripheral blood mononuclear cells (PBMCs), bone marrow, lymph nodes tissue, cord blood, thymus issue, tissue from a site of infection, ascites, pleural effusion, spleen tissue, or a combination thereof. In some embodiments, a subpopulation of primary T cells expressing TCRαβ, CD3, CD4, CD8, CD27 CD28, CD38, CD45RA, CD45RO, CD62L, CD127, CD122, CD95, CD197, CCR7, KLRG1, MHC-I proteins, MHC-II proteins, or a combination thereof may be further enriched, using a positive or negative selection technique, which is known in the art. In some embodiments, the T cell subpopulation express TCRαβ, CD4, CD8, or a combination thereof. In some embodiments, the T cell subpopulation express CD3, CD4, CD8, or a combination thereof. In some embodiments, the primary T cells for use in making the genetic edits disclosed herein may comprise at least 40%, at least 50%, or at least 60% CD27+CD45RO− T cells.

In some embodiments, parent T cells for use in making the genetically engineered CAR T cells (e.g., any of the T cells derived from primary T cell sources) may be undergone one or more rounds of stimulation, activation, expansion, or a combination thereof. In some embodiments, the parent T cells are activated and stimulated to proliferate in vitro before gene editing. In some embodiments, the T cells are activated, expanded, or both, before or after gene editing. In some embodiments, the T cells are activated and expanded at the same time as gene editing. In some embodiments, the T cells are activated and expanded for about 1-4 days, e.g., about 1-3 days, about 1-2 days, about 2-3 days, about 2-4 days, about 3-4 days, about 1 day, about 2 days, about 3 days, or about 4 days. In some embodiments, the allogeneic T cells are activated and expanded for about 4 hours, about 6 hours, about 12 hours, about 18 hours, about 24 hours, about 36 hours, about 48 hours, about 60 hours, or about 72 hours. Non-limiting examples of methods to activate and/or expand T cells are described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041.

(ii) CRISPR-Cas9-Mediated Gene Editing System

Any of the parent T cells may be subject to one or more genetic editing/modification steps to introduce the gene editing events disclosed herein, i.e., disrupt endogenous TRAC gene, disrupt endogenous β2M gene, and/or introducing a nucleic acid coding for any of the anti-BCMA CAR as disclosed herein. Conventional genetically engineering approaches, such as gene editing approaches (e.g., those disclosed herein) can be used. In some examples, the genetic modifications of the T cells can be implemented by a CRISPR/Cas9-mediated gene editing system.

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

TracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells, and occurs at a relatively low frequency in most cell types. In many embodiments of the present disclosure, NHEJ is utilized as the repair operant.

(a) Cas9

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 comprises a Streptococcus pyogenes-derived Cas9 nuclease protein that has been engineered to include C- and N-terminal SV40 large T antigen nuclear localization sequences (NLS). The resulting Cas9 nuclease (sNLS-spCas9-sNLS) is a 162 kDa protein that is produced by recombinant E. coli fermentation and purified by chromatography. The spCas9 amino acid sequence can be found as UniProt Accession No. Q99ZW2, which is provided herein as SEQ ID NO: 61.

Amino acid sequence of Cas9 nuclease (SEQ ID NO:
61):
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

(b) Guide RNAs (gRNAs)

CRISPR-Cas9-mediated gene editing as described herein includes the use of a guide RNA or a gRNA. As used herein, a “gRNA” refers to a genome-targeting nucleic acid that can direct the Cas9 to a specific target sequence within a TRAC gene or a β2M gene for gene editing at the specific target sequence. A guide RNA comprises at least a spacer sequence that hybridizes to a target nucleic acid sequence within a target gene for editing, and a CRISPR repeat sequence.

Exemplary gRNAs targeting a TRAC gene may comprise a nucleotide sequence provided in any one of SEQ ID NOs: 1-4. See WO 2019/097305A2, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154; Ensembl; ENSG00000277734). In some embodiments, gRNAs targeting the TRAC genomic region and Cas9 create breaks in the TRAC genomic region resulting Indels in the TRAC gene disrupting expression of the mRNA or protein.

Exemplary gRNAs targeting a β2M gene may comprise a nucleotide sequence provided in any one of SEQ ID NOs: 5-8. See also WO 2019/097305A2, the relevant disclosures of which are incorporated by reference herein for the purpose and subject matter referenced herein. Other gRNA sequences may be designed using the β2M gene sequence located on Chromosome 15 (GRCh38 coordinates: Chromosome 15: 44,711,477-44,718,877; Ensembl: ENSG00000166710). In some embodiments, gRNAs targeting the β2M genomic region and RNA-guided nuclease create breaks in the β2M genomic region resulting in Indels in the β2M gene disrupting expression of the mRNA or protein.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

The “target sequence” is in a target gene that is adjacent to a PAM sequence and is the sequence to be modified by Cas9. The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence.

For example, if the TRAC target sequence is 5′-AGAGCAACAGTGCTGTGGCC-3′ (SEQ ID NO: 10), then the gRNA spacer sequence is 5′-AGAGCAACAGUGCUGUGGCC-3′ (SEQ ID NO: 4). In yet another example, if the β2M target sequence is 5′-GCTACTCTCTCTTTCTGGCC-3′ (SEQ ID NO: 12), then the gRNA spacer sequence is 5′-GCUACUCUCUCUUUCUGGCC-3′ (SEQ ID NO: 8). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM.

A spacer sequence in a gRNA is a sequence (e.g., a 20 nucleotide sequence) that defines the target sequence (e.g., a DNA target sequences, such as a genomic target sequence) of a target gene of interest. An exemplary spacer sequence of a gRNA targeting a TRAC gene is provided in SEQ ID NO: 4. An exemplary spacer sequence of a gRNA targeting a β2M gene is provided in SEQ ID NO: 8.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

Non-limiting examples of gRNAs that may be used as provided herein are provided in WO 2019/097305A2, and WO/2019/215500, the relevant disclosures of each of the prior applications are herein incorporated by reference for the purposes and subject matter referenced herein. For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be a sgRNA, which may comprise a 20 nucleotide spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence.

In some embodiments, the sgRNA comprises no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as an sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

It should be understood that more than one suitable Cas9 and more than one suitable gRNA can be used in methods described herein, for example, those known in the art or disclosed herein. In some embodiments, methods comprise a Cas9 enzyme and/or a gRNA known in the art. Examples can be found in, e.g., WO 2019/097305A2, and WO/2019/215500, the relevant disclosures of each of the prior applications are herein incorporated by reference for the purposes and subject matter referenced herein.

(iii) AAV Vectors for Delivery of CAR Constructs to T Cells

A nucleic acid encoding any of the anti-BCMA CAR construct can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV for use in delivering the CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

A nucleic acid encoding an anti-BCMA CAR can be designed to insert into a genomic site of interest in the host T cells. In some embodiments, the target genomic site can be in a safe harbor locus.

In some embodiments, a nucleic acid encoding an anti-BCMA CAR (e.g., via a donor template, which can be carried by a viral vector such as an adeno-associated viral (AAV) vector) can be designed such that it can insert into a location within a TRAC gene to disrupt the TRAC gene in the genetically engineered T cells and express the CAR polypeptide. Disruption of TRAC leads to loss of function of the endogenous TCR. For example, a disruption in the TRAC gene can be created with an endonuclease such as those described herein and one or more gRNAs targeting one or more TRAC genomic regions. Any of the gRNAs specific to a TRAC gene and the target regions can be used for this purpose, e.g., those disclosed herein.

In some examples, a genomic deletion in the TRAC gene and replacement by an anti-BCMA CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions, and inserting a CAR coding segment into the TRAC gene.

A donor template as disclosed herein can contain a coding sequence for an anti-BCMA CAR. In some examples, the anti-BCMA CAR-coding sequence may be flanked by two regions of homology to allow for efficient HDR at a genomic location of interest, for example, at a TRAC gene using CRISPR-Cas9 gene editing technology. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing. Examples of the donor template, including flanking homology sequences, are provided in Table 4 below.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, can be inserted at a site nearby an endogenous promoter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EF1α promoter. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

The resultant T cells expressing an anti-BCMA CAR and having a disrupted TRAC and/or β2M genes may be collected and expanded in vitro. In some examples, the resultant T cells are subject to further purification to enrich the cells having the desired genetic modifications. For example, CAR⁺ T cells can be positively selected and TCR⁺ and/or B2M⁺ T cells can be excluded. In some embodiments, TCR⁺ T cells are removed. Non-limiting examples of methods of removal include cell sorting (e.g., fluorescence-activated cell sorting), immunomagnetic separation, chromatography, or microfluidic cell sorting. In some embodiments, TCR⁺ cells are removed using immunomagnetic separation. In some embodiments, TCR⁺ cells are labeled using a biotinylated antibody targeting the TCR and removed using anti-biotin magnetic beads.

(iv) Characterization of the Genetically Engineered Anti-BCMA CAR-T Cells

The genetically engineered anti-BCMA CAR-T cells, prepared by the methods disclosed herein or common approaches, can be characterized by routine approaches for features such as levels of surface protein of interest (e.g., TCR, β2M, anti-BCMA CAR, or a combination thereof), cell viability, cell bioactivity, impurity, etc.

In some embodiments, the surface protein of interest can be labeled, e.g., with an antibody and a tag such as a fluorescent tag. Flow cytometry can be used to detect the presence of the surface protein of interest, to quantify the level of surface marker expression, to quantify the fraction of T cells expressing the surface marker, or a combination thereof.

In some embodiments, insertion of the anti-BCMA CAR into the TRAC gene is assessed using digital droplet PCR (ddPCR). Digital PCR quantifies DNA concentration in a sample, comprising a) fractionating a PCR reaction; b) PCR amplifying the fractions; and c) analyzing the PCR amplifications of the fractions, wherein a fraction comprising a probe and a target molecule yields an amplification product and a fraction comprising no PCR probe yields no amplification product. The fraction containing amplification products is fitted to a Poisson distribution to determine the absolute copy number of target DNA molecules per given volume of the unfractionated sample (i.e., copies per microliter of sample) (see Hindson, B. et al., (2011) Anal Chem. 83:8604-10). Digital droplet PCR is a variation of digital PCR that can be used to provide absolute quantifications of DNA in samples, analyze copy number variations, and/or assess gene editing efficiencies. The sample of nucleic acids is fractionated into droplets using a water-oil emulsion; the PCR amplification is performed on the droplets collectively; and a fluidics system is used to separate the droplets and provide analysis of each individual droplet. In some embodiments, ddPCR is used to determine an absolute quantification of anti-BCMA CAR copies per sample composition. In some embodiments, ddPCR is used to assess HDR efficiency of inserting the anti-BCMA CAR sequences into the TRAC gene.

In some embodiments, the genetically engineered anti-BCMA CAR T cells can be assessed for cytokine-independent proliferation. The T cells are expected to only proliferate in the presence of a stimulatory cytokine, and proliferation in the absence of the stimulatory cytokine is indicative of a tumorigenic potential. The T cells may be cultured in the presence of a stimulatory cytokine for at least 1 day, e.g., at least 2, 3, 4, 5, 6, 7, 8, 9, 10, 11, 12, 13, 14, 15, 16, 17, 18, 19, or 20 days, and proliferation of the T cells can be determined by conventional approaches. In some examples, the stimulatory cytokine comprises IL-2, IL-7, or both. T cell proliferation may be assessed at the end of the culture period. Alternatively, T cell proliferation may be assessed during the culture period, for example, on the 1^{st}, 2^{nd}, 3^{rd}, 4^{th}, 5^{th}, or 6^{th} day of the culture period. In some examples, T cell proliferation can be assessed about every 1 day, about every 2 days, about every 3 days, about every 4 days, about every 5 days, about every 6 days, about every 7 days, or about every 8 days.

In some embodiments, viable T cells can be counted using a conventional method, for example, flow cytometry, microscopy, optical density, metabolic activity, or a combination thereof. In some embodiments, the genetically engineered anti-BCMA CAR-T cells disclosed herein do not proliferate in the absence of any of the stimulatory cytokines or a combination thereof (and is defined as lacking tumorigenic potential). No proliferation can be defined as the number of viable T cells at the end of the culture period being less than 150% of the number of viable T cells at the beginning of the culture period, e.g., less than 140%, less than 130%, less than 120%, less than 110%, less than 100%, less than 90%, less than 80%, less than 70%, less than 60%, less than 50%, less than 40%, less than 30%, less than 20%, or less than 10%.

In some embodiments, a population of the genetically modified anti-BCMA CAR-T cells disclosed herein may show no growth in the absence of one or more stimulatory cytokines when assessed at 10 days, 11 days, 12 days, 13 days, 14 days, 15 days, 16 days, 17 days, 18 days, 19 days or 20 days following culture. In some examples, the T cells do not proliferate in the absence of cytokine, growth factor, antigen, or a combination thereof.

### III. Allogeneic Anti-BCMA CAR-T Cell Therapy

Any of the genetically engineered anti-BCMA CAR-T cells disclosed herein may be used for therapeutic purposes, for example, in treating BCMA⁺ cancers. Accordingly, provided herein are methods of treating cancer (e.g., hematologic malignancies involving BCMA⁺ cancer cells) comprising administering an effective amount of a population of the genetically engineered anti-BCMA CAR-T cells disclosed herein (e.g., CTX120 cells) to a subject in need of the treatment. In some embodiments, the cancer is MM, including refractory and/or relapsed MM. MM is a malignancy of terminally differentiated plasma cells in the bone marrow. MM is a result of secretion of monoclonal immunoglobulin protein (e.g., M protein or monoclonal protein) or monoclonal free light chains by abnormal plasma cells. MM exists on a spectrum of plasma cell dyscrasias and results from the stepwise progression from premalignant monoclonal gammopathy of undetermined significance (MGUS) to smoldering (asymptomatic) MM to symptomatic MM. Symptoms of active MM include fatigue, low platelet count, frequent infections and/or fevers, bone damage, pain, kidney malfunction.

(i) Patient Population

The subject to be treated by the allogenic T cell therapy disclosed herein can be a mammal, for example, a human patient, who may be 18 years or older. In some examples, the subject is a human patient having a cancer that involves BCMA⁺ cancer cells. For example, the subject may be a human patient having MM, including symptomatic MM and asymptomatic MM. In specific examples, the human patient has refractory MM. In other specific examples, the human patient has relapsed MM. In other examples, the subject may have monoclonal gammopathy of unknown significance (MUGS) or asymptomatic smoldering MM. Alternatively, the subject may be a human patient who is diagnosed with a high risk of developing MM, e.g., a subtype disclosed herein such as symptomatic MM.

A subject having MM can be diagnosed via routine medical practice. Methods of diagnosing MM are known in the art. Non-limiting examples include analysis of bone marrow biopsy, analysis of end organ damage related to plasma cell proliferation (e.g., hypercalcemia, renal insufficiency, anemia, destructive bone lesions), or both. See e.g., Kumar, et al. (2017) Leukemia 31:2443-48; Kumar, et al., (2016) Lancet Oncol 17: e328-46; and NCCN Guidelines v.2.2019 (2018) National Comprehensive Cancer Network Clinical Practice Guidelines for Multiple Myeloma. In some embodiments, the subject has MGUS.

In some embodiments, the subject (e.g., a human patient) has MM cells expressing an elevated level of BCMA. Methods of quantifying expression of BCMA mRNA and protein in cells or tissues are known in the art. For example, expression of BCMA mRNA can be measured using reverse transcription polymerase chain reaction (RT-PCR), quantitative PCR (qPCR), multiplex-PCR, digital PCR, and/or whole transcriptome shotgun sequencing; and expression of BCMA protein can be measured using mass spectrometry, enzyme-linked immunosorbent assay (ELISA), protein immunoprecipitation, immunoelectrophoresis, western blot, and/or immunostaining (e.g., immunofluorescence staining, immunohistochemical staining) with analysis by flow cytometry or microscopy.

In some embodiments, the subject (e.g., a human patient) has relapsed from or is refractory to a prior MM therapy. As used herein, “refractory” refers to MM that does not respond to or becomes resistant to a treatment. As used herein, “relapsed” or “relapses” refers to MM that returns or progresses following a period of improvement (e.g., a partial or complete response) with treatment. In some embodiments, relapse occurs during the treatment. In some embodiments, relapse occurs after the treatment. A lack of response may be measured, for example, as a lack of change in serum M-protein levels, urine M-protein levels, bone marrow plasma cell counts, bone lesion sizes, bone lesion numbers, or a combination thereof. A return or progression in MM may be measured, for example, as an increase in serum creatinine levels, serum M-protein levels, urine M-protein levels, bone marrow plasma cell counts, bone marrow plasmacytomas sizes, bone marrow plasmacytomas numbers, bone lesion sizes, bone lesion numbers, calcium levels unexplained by other conditions, red blood cell counts, organ damage, or a combination thereof.

In some embodiments, the prior MM therapy comprises a steroid, chemotherapy, a proteasome inhibitor (PI), an immunomodulatory drug (IMiD), a monoclonal antibody, an autologous stem cell transplant (SCT), or a combination thereof (see e.g., NCCN Guidelines v.2.2019 (2018) National Comprehensive Cancer Network Clinical Practice Guidelines for Multiple Myeloma). Non-limiting examples of steroids include dexamethasone and prednisone. Non-limiting examples of chemotherapies include bendamustine, cisplatin, cyclophosphamide, doxorubicin hydrochloride, doxorubicin hydrochloride liposome, etoposide, and melphalan. Non-limiting examples of PIs include bortezomib, ixazomib, and carfilzomib. In some embodiments, the PI comprises bortezomib, carfilzomib, or both. Non-limiting examples of IMiDs include lenalidomide, pomalidomide, and thalidomide. In some embodiments, the IMiD therapy comprises lenalidomide, pomalidomide, or both. Non-limiting examples of monoclonal antibodies include CD38-directed monoclonal antibodies (e.g., daratumumab, and isatuximab), and elotuzumab (binding to CD319). In some embodiments, the monoclonal antibody comprises a CD38-directed monoclonal antibody such as daratumumab.

In some embodiments, the prior MM therapy comprises more than one line of therapy. In some embodiments, the prior MM therapy comprises two or more lines of therapy, e.g., three lines of prior therapy, four lines of prior therapy, etc. In some embodiments, the two or more lines of therapy are administered separately. In some embodiments, the two or more lines of therapy are administered in combination. In some embodiments, the prior MM therapy comprises an IMiD, a PI, a CD38-directed monoclonal antibody, or a combination thereof. In some embodiments, the prior MM therapy comprises IMiD and PI. In some embodiments, the IMiD is administered before the PI. In some embodiments, the IMiD is administered after the PI.

In some examples, the prior MM therapy comprises two lines of therapy, e.g., an IMiD, and a PI. A MM patient who is refractory to two prior MM therapies may be referred to as “double-refractory.” In some embodiments, a double-refractory MM patient has disease progression on or within 60 days of treatment with the two lines of therapy. In some instances, the two lines of therapy may be part of the same regimen. In other instances, the two lines of therapy may be part of different treatment regimens. A double-refractory MM patient may have disease progression on or within 60 days of the last treatment regimen.

In some embodiments, the prior MM therapy comprises three lines of therapy, e.g., an IMiD, a PI, and a CD38-directed monoclonal antibody. A MM patient who is refractory to three prior MM therapies may be referred to as “triple-refractory.” In some embodiments, a triple-refractory MM patient has disease progression on or within 60 days of treatment with the three lines of therapy. In some instances, the three lines of therapy may be part of the same regimen. In other instances, the three lines of therapy may be part of different treatment regimens. A triple-refractory MM patient may have disease progression on or within 60 days of the last treatment regimen.

In some embodiments, relapsed or refractory MM is detected at least 10 days, at least 20 days, at least 30 days, at least 2 months, at least 4 months, at least 6 months, at least 8 months, at least 10 months, at least 1 year, at least 2 years, at least 3 years, at least 4 years, or at least 5 years after the prior MM therapy. In some embodiments, relapsed or refractory MM is detected within 10-100 days after the prior MM therapy, e.g., within 10-90 days, 20-90 days, 20-80 days, 30-80 days, 30-70 days, 40-70 days, 40-60 days, or 50-60 days. In some embodiments, relapsed or refractory MM is detected within about 100 days after the prior MM therapy, e.g., within about 90 days, within about 80 days, within about 70 days, within about 60 days, within about 50 days, within about 40 days, within about 30 days, within about 20 days, or within about 10 days after the prior MM therapy.

In some embodiments, relapsed MM is detected in the subject during an autologous SCT. In some embodiments, relapsed MM is detected in the subject after an autologous SCT. In some embodiments, relapsed or refractory MM is detected at least 10 days, at least 20 days, at least 30 days, at least 2 months, at least 4 months, at least 6 months, at least 8 months, at least 10 months, at least 1 year, at least 2 years, at least 2 years, at least 3 years, at least 4 years, or at least 5 years after the autologous SCT. In some embodiments, relapsed or refractory MM is detected within about 18 months after the autologous SCT, e.g., within about 17 months, within about 16 months, within about 15 months, within about 14 months, within about 13 months, within about 12 months, within about 11 months, within about 10 months, within about 9 months, within about 8 months, within about 7 months, within about 6 months, within about 5 months, within about 4 months, within about 3 months, within about 2 months, or within about 1 month after the autologous SCT. In some embodiments, relapsed or refractory MM is detected between about 1-18 months after the autologous SCT, e.g., about 2-18 months, about 2-16 months, about 3-16 months, about 3-14 months, about 4-14 months, about 4-12 months, about 5-12 months, about 5-10 months, about 6-10 months, or about 6-8 months after the autologous SCT.

In some embodiments, the subject is a human MM patient having one or more of the following features: adequate organ function, free of a prior allogeneic stem cell transplantation (SCT), free of autologous SCT within 60 days prior to the enrollment into the allogenic T cell therapy disclosed herein, free of plasma cell leukemia, non-secretory MM, Waldenstrom's macroglobulinemia, POEM syndrome, and/or amyloidosis with end organ involvement and damage, free of prior gene therapy, anti-BCMA therapy, and non-palliative radiation therapy within 14 days prior to enrollment into the allogenic T cell therapy, free of central nervous system involvement by MM, free of history or presence of clinically relevant CNS pathology, cerebrovascular ischemia and/or hemorrhage, dementia, a cerebellar disease, an autoimmune disease with CNS involvement, free of unstable angina, arrhythmia, and/or myocardial infarction within 6 month prior to enrollment into the allogenic T cell therapy, free of uncontrolled infections (e.g., infections is caused by HIV, HBV, or HCV), free of previous or concurrent malignancy, provided that the malignancy is not basal cell or squamous cell skin carcinoma, adequately resected and in situ carcinoma of cervix, or a previous malignancy that was completely resected and has been in remission for ≥5 years, free of live vaccine administration within 28 days prior to enrollment into the allogenic T cell therapy, free of systemic anti-tumor therapy within 14 days prior to enrollment into the allogenic T cell therapy, and free of primary immunodeficiency disorders or autoimmune disorders that require immunosuppressive therapy. In some embodiments, the subject is a human patient having Eastern Cooperative Oncology Group (ECOG) performance status of 0 or 1. The human patient may be free of contraindication to lymphodepleting agents such as cyclophosphamide and/or fludarabine.

In some examples, the subject is a human patient who meets one or more of the inclusion and/or exclusion criteria disclosed in Example 6 below. In some examples, the subject may meet all of the inclusion and/or exclusion criteria disclosed in Example 6 below.

(ii) Conditioning Regimen (Lymphodepleting Therapy)

Any human patients suitable for the allogeneic anti-BCMA CAR-T cell therapy as disclosed herein may receive a lymphodepleting therapy prior to infusion of the anti-BCMA CAR-T cells to reduce or deplete the endogenous lymphocyte of the subject.

Lymphodepletion (LD) refers to the destruction of endogenous lymphocytes and/or T cells, which is commonly used prior to immunotransplantation and immunotherapy. Lymphodepletion can be achieved by irradiation and/or chemotherapy. A “lymphodepleting agent” can be any molecule capable of reducing, depleting, or eliminating endogenous lymphocytes and/or T cells when administered to a subject. In some embodiments, the lymphodepleting agents are administered in an amount effective in reducing the number of lymphocytes by at least 10%, 20%, 30%, 40%, 50%, 60%, 70%, 80%, 90%, 95%, 96%, 96%, 97%, 98%, or at least 99% as compared to the number of lymphocytes prior to administration of the agents. In some embodiments, the lymphodepleting agents are administered in an amount effective in reducing the number of lymphocytes such that the number of lymphocytes in the subject is below the limits of detection. In some embodiments, the subject is administered at least one (e.g., 2, 3, 4, 5 or more) lymphodepleting agents.

In some embodiments, the lymphodepleting agents are cytotoxic agents that specifically kill lymphocytes. Examples of lymphodepleting agents include, without limitation, fludarabine, cyclophosphamide, bendamustin, 5-fluorouracil, gemcitabine, methotrexate, dacarbazine, melphalan, doxorubicin, vinblastine, cisplatin, oxaliplatin, paclitaxel, docetaxel, irinotecan, etopside phosphate, mitoxantrone, cladribine, denileukin diftitox, or DAB-IL2. In some instances, the lymphodepleting agent may be accompanied with low-dose irradiation. The lymphodepletion effect of the conditioning regimen can be monitored via routine practice.

In some embodiments, the method described herein involves a conditioning regimen that comprises one or more lymphodepleting agents, for example, fludarabine and cyclophosphamide. A human patient to be treated by the method described herein may receive multiple doses of the one or more lymphodepleting agents for a suitable period (e.g., 1-5 days) in the conditioning stage. The patient may receive one or more of the lymphodepleting agents once per day during the lymphodepleting period. In one example, the human patient receives fludarabine at about 20-50 mg/m² (e.g., 30 mg/m²) per day for 2-4 days (e.g., 3 days) and cyclophosphamide at about 300-600 mg/m² (e.g., 500 mg/m²) per day for 2-4 days (e.g., 3 days).

In one example, the human patient receives fludarabine at about 30 mg/m² per day for 3 days and cyclophosphamide at about 300 mg/m² per day for 3 days. In other examples, the human patient receives fludarabine at about 30 mg/m² per day for 3 days and cyclophosphamide at about 500 mg/m² per day for 3 days.

In some embodiments, the LD chemotherapy increases a serum level of IL-7, IL-15, IL-2, IL-21, IL-10, IL-5, IL-8, MCP-1, PLGF, CRP, sICAM-1, sVCAM-1, or a combination thereof in the subject. In some embodiments, the LD chemotherapy decreases a serum level of perforin, MIP-1b, or both in the subject. In some embodiments, the LD chemotherapy is associated with lymphopenia in the subject. In some embodiments, the LD chemotherapy is associated with a decrease of regulatory T cells in the subject.

Before the LD chemotherapy, the subject may be examined for conditions that may suggest delay of the LD chemotherapy. Exemplary conditions include: significant worsening of clinical status, requirement for supplemental oxygen to maintain a saturation level of greater than about 90%, uncontrolled cardiac arrhythmia, hypotension requiring vasopressor support, active infection, and/or grade≥2 acute neurological toxicity. If one or more of the conditions occur, LC chemotherapy to a subject should be delayed until improvement of the conditions.

(iii) Allogenic Anti-BCMA CAR-T Cell Therapy

After a subject has been conditioned for receiving allogenic CAR-T cell therapy (e.g., have undergone the LD chemotherapy), an effective amount of the population of genetically engineered anti-BCMA CAR-T cells (e.g., CTX120 cells) or a pharmaceutical composition comprising such as disclosed herein (e.g., comprising CTX120 cells suspended in a cryopreservation solution, which may comprise about 5% DMSO) may be given to the subject (e.g., a human MM patient) via suitable route and schedule. In some examples, the T cells are administered via intravenous infusion. “Allogenic T cell therapy” means that the T cells given to a recipient is derived from one or more donors of the species but not from the recipient. In the allogenic cell therapy disclosed herein, the genetically engineered anti-BCMA CAR-T cells (e.g., CTX120 cells) may be derived from one or more health human donors and are given to a human MM patient.

In some embodiments, the genetically engineered anti-BCMA CAR-T cells (e.g., CTX120 cells) can be administered to a subject (e.g., a human MM patient) at least 24 hours (one day) after the subject receives the LD chemotherapy. For example, administration of the genetically engineered anti-BCMA CAR-T cells (e.g., CTX120 cells) may be 2-7 days after the LD chemotherapy. In some embodiments, the allogeneic T cells are administered no more than ten days after administration of the LD chemotherapy, e.g., no more than nine days, no more than eight days, no more than seven days, no more than six days, no more than five days, no more than four days, no more than three days, no more than two days, or no more than one day. In some embodiments, the allogeneic T cells are administered within 24 hours to ten days, 24 hours to nine days, 30 hours to nine days, 30 hours to eight days, 36 hours to eight days, 36 hours to seven days, or 48 hours to seven days, after administration of the LD chemotherapy. In some embodiments, the allogeneic T cells are administered within 48 hours to seven days after administration of the LD chemotherapy.

After the LD chemotherapy and before administration of the genetically engineered anti-BCMA CAR-T cells, the subject (e.g., a human MM patient) may be examined for conditions that may suggest delay of the allogenic T cell administration. Exemplary conditions include: active uncontrolled infection, worsening of clinical status compared to the clinical status prior to the LD chemotherapy, and/or grade≥2 acute neurological toxicity. Administration of the anti-BCMA CAR-T cells should be delayed if one or more of such conditions occur until improvement is observed. If the delay extends beyond a certain period after the LD chemotherapy (e.g., at least 10 days, at least 12 days, at least 15 days, or at least 21 days after the LD chemotherapy), the LD chemotherapy may be repeated before administration of the anti-BCMA CAR-T cells.

To perform the allogenic T cell therapy, an effective amount of the population of genetically engineered anti-BCMA CAR-T cells as disclosed herein, for example, CTX120 cells, can be administered to a suitable subject (e.g., a human MM patient), who meets the requirements disclosed herein. The genetically engineered anti-BCMA CAR-T cells (e.g., CTX120 cells) may be suspended in a cryopreservation solution, which may comprise about 2-10% DMSO (e.g., about 5% DMSO), and optionally substantially free of serum. As used herein, the term “an effective amount” refers to an amount sufficient to provide a desired effect in treating MM. Non-limiting examples of the desired effects include preventing development of MM; reducing likelihoods of developing MM; slowing, delaying, arresting or reversing progression of MM; inhibiting, reducing, ameliorating, or alleviating a symptom of MM, or a combination thereof in the subject. The effective amount of a given case can be determined by one of ordinary skill in the art using routine experimentation, for example, by accessing a change in a relevant target level (e.g., by at least 10%), need for hospitalization or other medical interventions.

In some embodiments, a population of genetically engineered anti-BCMA CAR-T cells such as CTX120 cells comprising about 2.5×10⁷ to about 7.5×10⁸ CAR⁺ T cells are administered to a human MM patient (e.g., those disclosed herein) via intravenous infusion. For example, about 5×10⁷ to about 7.5×10⁸ genetically engineered anti-BCMA CAR-T cells (e.g., CTX120) expressing the anti-BCMA CAR may be administered to the patient by intravenous infusion. Exemplary effective amount of CAR⁺ T cells for use in the allogenic T cell therapy disclosed herein include about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, or about 7×10⁸. In some examples, a population of genetically engineered anti-BCMA CAR-T cells such as CTX120 cells comprising about 2.5×10⁷ CAR⁺ T cells are administered to the patient by intravenous infusion.

In some examples, a population of genetically engineered anti-BCMA CAR-T cells such as CTX120 cells comprising about 5×10⁷ CAR⁺ T cells are administered to the patient by intravenous infusion. In some examples, a population of genetically engineered anti-BCMA CAR-T cells such as CTX120 cells comprising about 1.5×10⁸ CAR⁺ T cells are administered to the patient by intravenous infusion. In some examples, a population of genetically engineered anti-BCMA CAR-T cells such as CTX120 cells comprising about 4.5×10⁸ CAR⁺ T cells are administered to the patient by intravenous infusion. In some examples, a population of genetically engineered anti-BCMA CAR-T cells such as CTX120 cells comprising about 6×10⁸ CAR⁺ T cells are administered to the patient by intravenous infusion. In some examples, a population of genetically engineered anti-BCMA CAR-T cells such as CTX120 cells comprising about 7.5×10⁸ CAR⁺ T cells are administered to the patient by intravenous infusion.

In some embodiments, an effective amount of the genetically engineered T cell population as disclosed herein (e.g., the CTX120 cells) may range from about 1.5×10⁸ to about 7.5×10⁸ CAR⁺ T cells, for example, about 1.5×10⁸ to about 4.5×10⁸ CAR⁺ T cells or about 4.5×10⁸ to about 7.5×10⁸ CAR⁺ T cells. In some embodiments, an effective amount of the genetically engineered T cell population as disclosed herein (e.g., the CTX120 cells) may range from about 4.5×10⁸ to about 6×10⁸ CAR⁺ T cells, or about 6×10⁸ to about 7.5×10⁸ CAR⁺ T cells.

In some embodiments, the effective amount of the genetically engineered anti-BCMA CAR-T cells disclosed herein such as CTX120 cells is sufficient to decrease serum M-protein levels by at least 25% in the subject, e.g., by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, or by at least 95% in the subject.

In some embodiments, the effective amount of the genetically engineered anti-BCMA CAR-T cells disclosed herein such as CTX120 cells is sufficient to decrease 24-hour urine M-protein levels by at least 50% in the subject, e.g., by at least 55%, by at least 60%, by at least 65%, by at least 70%, by at least 75%, by at least 80%, by at least 85%, by at least 90%, or by at least 95% in the subject. In some embodiments, the effective dosage is sufficient to decrease serum M-protein levels by at least 25%, 24-hour urine M-protein levels by at least 50%, or both in the subject. In some embodiments, the effective dosage is sufficient to decrease serum M-protein levels by at least 25% and 24-hour urine M-protein levels by at least 50% in the subject. In some embodiments, the effective dosage is sufficient to decrease serum M-protein levels by at least 50%, 24-hour urine M-protein levels by at least 90%, or both in the subject. In some embodiments, the effective dosage is sufficient to decrease serum M-protein levels by at least 50% and 24-hour urine M-protein levels by at least 90% in the subject.

In some embodiments, the effective amount of the genetically engineered anti-BCMA CAR-T cells disclosed herein such as CTX120 cells is sufficient to decrease 24-hour urine M-protein levels to less than 200 mg in the subject, e.g., to less than 190 mg, to less than 180 mg, to less than 170 mg, to less than 160 mg, to less than 150 mg, to less than 140 mg, to less than 130 mg, to less than 120 mg, to less than 110 mg, to less than 100 mg, to less than 90 mg, to less than 80 mg, to less than 70 mg, to less than 60 mg, or to less than 50 mg in the subject. In some embodiments, the effective dosage is sufficient to decrease serum M-protein levels by at least 90%, 24-hour urine M-protein levels to less than 100 mg, or both in the subject. In some embodiments, the effective dosage is sufficient to decrease serum M-protein levels by at least 90% and 24-hour urine M-protein levels to less than 100 mg in the subject.

In some embodiments, the effective amount of the genetically engineered anti-BCMA CAR-T cells disclosed herein such as CTX120 cells is sufficient to decrease soft tissue plasmacytomas sizes (SPD) by at least 30% in the subject, e.g., by at least 35%, by at least 40%, by at least 45%, by at least 50%, by at least 55%, by at least 60%, by at least 65%, by at least 70%, by at least 75%, by at least 80%, by at least 85%, by at least 90%, or by at least 95% in the subject. In some embodiments, the effective dosage is sufficient to decrease soft tissue plasmacytomas sizes (SPD) by at least 50% in the subject. In some embodiments, the effective dosage is sufficient to decrease soft tissue plasmacytomas to undetectable levels.

In some embodiments, the effective amount of the genetically engineered anti-BCMA CAR-T cells disclosed herein such as CTX120 cells is sufficient to decrease plasma cell counts by at least 20% in the subject, e.g., by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, or by at least 95% in the subject. In some embodiments, the effective dosage is sufficient to decrease plasma cell counts by at least 50% in the subject.

In some embodiments, the effective amount of the genetically engineered anti-BCMA CAR-T cells disclosed herein such as CTX120 cells is sufficient to decrease plasma cell counts to less than 10% of bone marrow (BM) aspirates in the subject, e.g., less than 9%, less than 8%, less than 7%, less than 6%, less than 5%, less than 4%, or less than 3% of BM aspirates in the subject. In some embodiments, the effective dosage is sufficient to decrease plasma cell counts to less than 5% of BM aspirates in the subject. In some embodiments, the effective dosage is sufficient to decrease serum M-proteins, urine M-proteins, and soft tissue plasmacytomas to undetectable levels, and plasma cell counts to less than 5% of BM aspirates in the subject.

In some embodiments, the effective amount of the genetically engineered anti-BCMA CAR-T cells disclosed herein such as CTX120 cells is sufficient to decrease differences between involved and uninvolved free light chain (FLC) levels by at least 20% in the subject, e.g., by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, or by at least 95% in the subject. In some embodiments, the effective dosage is sufficient to decrease differences between involved and uninvolved FLC levels by at least 50% in the subject.

In some embodiments, the subject has myeloma cells that produce kappa (κ) light chains, and the effective dosage is sufficient to decrease kappa-to-lambda light chain ratios (κ/λ ratios) to 6:1 or lower, e.g., 11:2 or lower, 11:2 or lower, 5:1 or lower, 9:2 or lower, 4:1 or lower, 7:2 or lower, 3:1 or lower, 5:2 or lower, 2:1 or lower, 3:2 or lower, or 1:1 or lower. In some embodiments, the subject has myeloma cells that produce κ light chains, and the effective dosage is sufficient to decrease κ/λ ratios to 4:1 or lower.

In some embodiments, the subject has myeloma cells that produce lambda (λ) light chains, and the effective dosage is sufficient to increase kappa-to-lambda light chain ratios (κ/λ ratios) to 1:4 or higher, e.g., 2:7 or higher, 1:3 or higher, 2:5 or higher, 1:2 or higher, 1:1 or higher, 3:2 or higher, or 2:1 or higher. In some embodiments, the subject has myeloma cells that produce λ light chains, and the effective dosage is sufficient to increase κ/λ ratios to 1:2 or higher.

In some embodiments, one or more assays may be performed to a subject before and/or after the treatment by the anti-BCMA CAR-T cells disclosed herein (e.g., CTX120 cells) for measuring any of the disease status indicators as disclosed herein, for example, soft tissue plasmacytomas sizes (SPD), serum M-protein levels, urine M-protein levels, free light chain (FLC) levels, plasma cell counts, kappa-to-lambda light chain ratios, or a combination thereof. Routine laboratory tests can be used to measure such indicators. In some examples, the subject may be examined for levels of serum and/or urine monoclonal protein (M-protein) before the anti-BCMA CAR-T cell treatment, after the anti-BCMA CAR-T cell treatment, or both. Alternatively or in addition, the subject may be examined for free light chain (FLC) levels before and/or after the CAR-T cell treatment. Alternatively or in addition, the subject may be examined for bone marrow plasma cell counts before and/or after the CAR-T cell treatment.

In some embodiments, the effective amount of the genetically engineered anti-BCMA CAR-T cells disclosed herein such as CTX120 cells comprises 1×10⁶ or less TCR⁺ T cells/kg (subject), e.g., 8×10⁵ or less, 6×10⁵ or less, 4×10⁵ or less, 2×10⁵ or less, 1×10⁵ or less, 8×10⁴ or less, 6×10⁴ or less, 4×10⁴ or less, 2×10⁴ or less, or 1×10⁴ or less TCR⁺ T cells/kg (subject). In some embodiments, the effective dosage comprises about 1×10⁴ to about 1×10⁶ TCR⁺ T cells/kg (subject), e.g., about 1×10⁴ to about 1×10⁶, about 2×10⁴ to about 1×10⁶, about 2×10⁴ to about 8×10⁵, about 4×10⁴ to about 8×10⁵, about 4×10⁴ to about 6×10⁵, about 6×10⁴ to about 6×10⁵, about 6×10⁴ to about 4×10⁵, about 8×10⁴ to about 4×10⁵, or about 1×10⁵ to about 2×10⁵ TCR⁺ T cells/kg (subject). In some embodiments, the effective dosage comprises 1×10⁵ or less TCR⁺ T cells/kg (subject). In some embodiments, the effective dosage comprises 7×10⁴ or less TCR⁺ T cells/kg (subject).

In some embodiments, the genetically engineered anti-BCMA CAR-T cells disclosed herein such as CTX120 cells T cells are injected, for example, infused intravenously. Non-limiting examples of routes of administration include intravenous, intrathecal, intraperitoneal, intraspinal, intracereberal, spinal, and intrasternal infusion. In some embodiments, the route is intravenous. In some embodiments, the genetically engineered anti-BCMA CAR-T cells disclosed herein such as CTX120 cells are administered directly into a target site, tissue, or organ. In some embodiments, the genetically engineered anti-BCMA CAR-T cells disclosed herein such as CTX120 cells are administered systemically (e.g., into the subject's circulatory system). In some embodiments, the systemic route comprises intraperitoneal administration, intravenous administration, or both. In some embodiments, the genetically engineered anti-BCMA CAR-T cells disclosed herein such as CTX120 cells are administered as a single intravenous infusion. In some embodiments, the allogeneic T cells are administered as two or more intravenous infusions.

After the allogenic T cell therapy disclosed herein, the subject shall be monitored for development of acute toxicity, for example, cytokine release syndrome (CRS), neurotoxicity, tumor lysis syndrome, hemophagocytic lymphohistiocytosis (HLH), Cytopenias, GvHD, hypotention, renal insufficiency, viral encephalitis, neutropenia, thrombocytopenia or a combination thereof. Toxicity management known to those medical practitioners shall be performed to the subject if toxicity is observed after administration of the genetically engineered anti-BCMA CAR-T cells such as CTX120 cells. See Example 6 for more details regarding toxicity management.

In some instances, a pharmacokinetic (PK) profile of the genetically engineered anti-BCMA CAR-T cells such as CTX120 cells in a human recipient after administration may be examined. The PK profile may evaluate an effectiveness of the allogenic T cell therapy on a human MM patient.

The genetically engineered CAR-T cells may undergo an expansion phase following administration to a subject. Expansion is a response to antigen recognition and signal activation (Savoldo, B. et al. (2011) J Clin Invest. 121:1822; van der Stegen, S. et al. (2015) Nat Rev Drug Discov. 14:499-509). Following expansion, the genetically engineered CAR-T cells undergo a contraction phase, where short-lived effector CAR-T cells are eliminated and that are long-lived memory CAR-T cells remain. The duration of the persistence phase provides a measure of the longevity of the CAR-T cells following expansion and contraction.

In some embodiments, the PK profile comprises the quantity of the genetically engineered anti-BCMA CAR-T cells in a tissue over time. Exemplary tissues suitable for this analysis include peripheral blood. The tissue sample may be collected daily or weekly. Alternatively or in addition, the tissue sample may be collected starting on day 1, day 2, day 3, or day 4 after T cell administration. Collection of the tissue sample may end not earlier than day 5 after the T cell administration, e.g., not earlier than day 8, not earlier than day 10, not earlier than day 15, or not earlier than day 20 after T cell administration. In some embodiments, collection of the tissue sample is performed at least once per week after T cell administration, e.g., at least twice, or at least 3 times per week after T cell administration. In some embodiments, collection of the tissue sample is performed for up to 16 weeks after T cell administration, e.g., up to 15 weeks, up to 12 weeks, up to 10 weeks, up to 8 weeks, or up to 6 weeks.

In some embodiments, evaluating the PK profile comprising obtaining a baseline measurement, which may be obtained before administration of the genetically engineered anti-BCMA CAR T cells, for example, no more than 15 days before T cell administration, e.g., no more than 10 days, no more than 5 days, no more than 1 day before T cell administration. In some embodiments, the baseline measurement is obtained within 0.25 to 48 hours before T cell administration, e.g., within 0.5-24 hours, within 1 to 36 hours, within 1-12 hours, or within 2-12 hours.

In some embodiments, the time course of the quantity of the genetically engineered anti-BCMA CAR-T cells in the tissue is measured by an area under the curve (AUC). A method of calculating an AUC is known to one skilled in the art and is comprised of approximating an AUC by a series of trapezoids, computing the area of the trapezoids, and summing the area of the trapezoids to determine the AUC. In some embodiments, an AUC is defined for a PK profile wherein the quantity of the genetically engineered anti-BCMA CAR-T cells is measured for a given tissue type over time. In some embodiments, an AUC is defined for a PK profile from one designated time point to another designated time point (i.e., AUC10-80 refers to the total area under a quantity-time curve depicting quantity from day 10 to day 80 following administration). In some embodiments, an AUC is determined for a preselected time period extending from time of administration (e.g., day 1) to a time ending on a day that is 1-7, 10-20 days, 15-45 days, 20-70 days, 25-100 days, or 40-180 days following administration. In some embodiments, an AUC measured for a PK profile in a recipient is indicative of a response in the recipient (e.g., CR or PR). In some embodiments, an AUC measured for a PK profile in a recipient is indicative of a risk of relapse in the recipient.

In some embodiments, the genetically engineered anti-BCMA CAR-T cells do not induce toxicity in non-cancer cells in the subject. Alternatively, the genetically engineered anti-BCMA CAR-T cells do not trigger complement mediated lysis, or does not stimulate antibody-dependent cell mediated cytotoxicity (ADCC).

In some embodiments, the allogenic anti-BCMA CAR-T cell therapy may be in combination with one or more anti-cancer therapies, for example, therapies commonly applied to multiple myeloma.

### IV. Kit for Allogeneic Anti-BCMA CAR-T Cell Therapy

The present disclosure also provides kits for use of a population of anti-BCMA CAR T cells such as CTX120 T cells as described herein in methods for treating multiple myeloma, such as refractory and/or relapsed multiple myeloma. Such kits may include a first container comprising a first pharmaceutical composition that comprises any of the populations of genetically engineered anti-BCMA CAR T cells (e.g., those described herein such as CTX120 cells), and a pharmaceutically acceptable carrier. The anti-BCMA CAR-T cells may be suspended in a cryopreservation solution such as those disclosed herein. Optionally, the kit may further comprise a second container comprising a second pharmaceutical composition that comprises one or more lymphodepleting agents.

In some embodiments, the kit can comprise instructions for use in any of the methods described herein. The included instructions can comprise a description of administration of the first and/or second pharmaceutical compositions to a subject to achieve the intended activity in a human MM patient. The kit may further comprise a description of selecting a human MM patient suitable for treatment based on identifying whether the human patient is in need of the treatment. In some embodiments, the instructions comprise a description of administering the first and second pharmaceutical compositions to a human patient who is in need of the treatment.

The instructions relating to the use of a population of anti-BCMA CAR-T cells such as CTX120 T cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the population of genetically engineered T cells is used for treating, delaying the onset, and/or alleviating a symptom of MM in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of the anti-BCMA CAR-T cells such as the CTX120 T cells as disclosed herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984»; Animal Cell Culture (R. I. Freshney, ed. (1986; Immobilized Cells and Enzymes (IRL Press, (1986; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Preparation of Anti-BCMA CAR T Cells

Genetically engineered T cells expressing a CAR specific for the BCMA antigen (e.g., CTX120 cells) were prepared from healthy donor PBMCs obtained via a standard leukapheresis procedure as described in WO2019/097305 and WO/2019/215500, the relevant disclosures of each of which are incorporated by reference for the purpose and subject matter referenced herein.

Briefly, mononuclear cells were enriched for T cells and activated with anti-CD3/CD28 antibody-coated beads. The enriched and activated T cells were then genetically modified using CRISPR/Cas9 to disrupt (e.g., generate a gene knockout) the coding sequences of the TRAC gene and the β2M gene, with simultaneous insertion of a CAR specific to BCMA that is expressed by human MM cells. The insertion of the CAR occurred by HDR of a DNA DSB generated by Cas9/gRNA. The CAR was encoded by donor DNA with left and right flanking homology arms that were specific to the TRAC gene, thus enabling insertion of the CAR into a DNA DSB generated at the TRAC gene. The CAR homology donor DNA was administered using rAAV6. Disruption of the TRAC gene yielded loss of function of the TCR and renders the gene-edited T cell non-alloreactive and suitable for allogeneic transplantation by minimizing the risk of GVHD, while disruption of the β2M gene yielded loss of expression of MHC I and prevents susceptibility of the gene-edited T cells to a HVG response. Insertion of an anti-BCMA CAR into the TRAC gene provides T cells that are reactive to MM tumor cells that express BCMA surface antigen.

To perform the gene-editing, primary human T cells were first electroporated with Cas9-sgRNA RNP complexes targeting the TRAC and β2M genes. Cas9 nuclease was mixed with TA-1 sgRNA (SEQ ID NO:1, targeting TCR) and with B2M-1 sgRNA (SEQ ID NO:5, targeting β2M) in separate microcentrifuge tubes. Each solution was incubated for no less than 10 minutes at room temperature to form each ribonucleoprotein complex. The two Cas9/gRNA mixtures were combined, and mixed with the cells, bringing Cas9, TA-1 and B2M-1 to a final concentration of 0.3 mg/mL, 0.08 mg/mL and 0.2 mg/mL, respectively. Cells were electroporated with the Cas9-sgRNA RNP. Following electroporation, cells were treated with rAAV6 encoding an anti-BCMA CAR with flanking left and right 800-bp homology arms specific to the TRAC locus. The encoded CAR was operably linked to a 5′ elongation factor EF-1α to function as a promoter and a 3′ polyadenylation sequence to promote mRNA transcription stability. The CAR comprised a humanized scFv derived from a murine antibody specific for human BCMA, a hinge region and transmembrane domain, a signaling domain comprising CD3ζ, and a 4-1BB co-stimulatory domain.

The target gene sequences, and sgRNAs, and the spacer sequences encoded by the sgRNAs are provided in Table 1.

TABLE 1
sgRNA Sequences and Target Gene Sequences

SEQ ID
NO:

sgRNA Sequences

| | | | |
|---|---|---|---|
| TRAC sgRNA | Modified | A*G*A*GCAACAGUGCUGUGGCCguuuuagagcuagaaauagcaaguuaa | 1 |
| | | aauaaggcuaguccguuaucaacuugaaaaaguggcaccgagucggugcU*U*U*U | |
| | Unmodified | AGAGCAACAGUGCUGUGGCCguuuuagagcuagaaauagcaaguuaaaau | 2 |
| | | aaggcuaguccguuaucaacuugaaaaaguggcaccgagucggugcUUUU | |
| TRAC sgRNA | Modified | A*G*A*GCAACAGUGCUGUGGCC | 3 |
| spacer | Unmodified | AGAGCAACAGUGCUGUGGCC | 4 |
| β2M sgRNA | Modified | G*C*U*ACUCUCUCUUUCUGGCCguuuuagagcuagaaauagcaaguuaa | 5 |
| | | aauaaggcuaguccguuaucaacuugaaaaaguggcaccgagucggugcU*U*U*U | |
| | Unmodified | GCUACUCUCUCUUUCUGGCCguuuuagagcuagaaauagcaaguuaaaau | 6 |
| | | aggcuaguccguuaucaacuugaaaaaguggcaccgagucggugcUUUU | |
| β2M sgRNA | Modified | G*C*U*ACUCUCUCUUUCUGGCC | 7 |
| spacer | Unmodified | GCUACUCUCUCUUUCUGGCC | 8 |

Target Sequences (PAM)

| | | |
|---|---|---|
| TRAC sgRNA | AGAGCAACAGTGCTGTGGCC(TGG) | 9 |
| TRAC sgRNA | AGAGCAACAGTGCTGTGGCC | 10 |
| β2M sgRNA | GCTACTCTCTCTTTCTGGCC(TGG) | 11 |
| β2M sgRNA | GCTACTCTCTCTTTCTGGCC | 12 |

A disrupted TRAC gene produced by a TRAC sgRNA in Table 1 above may comprise one of the edited TRAC gene sequences provided in Table 2 below (“-” indicates deletion and residues in boldface indicate mutation or insertion):

TABLE 2
Edited TRAC Gene Sequence

| | Sequence (Deletions indicated by dashes (-); | |
|---|---|---|
| Description | insertions indicated by bold) | SEQ ID NO: |
| TRAC gene edit | AA---------------------GAGCAACAAATCTGACT | 13 |
| TRAC gene edit | AAGAGCAACAGTGCTGT-GCCTGGAGCAACAAATCTGACT | 14 |
| TRAC gene edit | AAGAGCAACAGTG-------CTGGAGCAACAAATCTGACT | 15 |
| TRAC gene edit | AAGAGCAACAGT------GCCTGGAGCAACAAATCTGACT | 16 |
| TRAC gene edit | AAGAGCAACAGTG---------------------CTGACT | 17 |
| TRAC gene edit | AAGAGCAACAGTGCTGTGGGCCTGGAGCAACAAATCTGACT | 18 |
| TRAC gene edit | AAGAGCAACAGTGC--TGGCCTGGAGCAACAAATCTGACT | 19 |
| TRAC gene edit | AAGAGCAACAGTGCTGTGTGCCTGGAGCAACAAATCTGACT | 20 |

A portion of the genetically engineered anti-BCMA CAR-T cells may comprise an edited TRAC gene, a fragment of which may be replaced by the nucleotide sequence encoding the anti-BCMA CAR via homologous recombination at the regions corresponding to the left and right homology arms (see Table 4 below). As such, a portion of the genetically engineered anti-BCMA CAR-T cells disclosed herein (e.g., CTX120 cells) may comprise a disrupted TRAC gene, which has a deletion of at least the AGAGCAACAGTGCTGTGGCC (SEQ ID NO: 10) fragment. A nucleic acid comprising a nucleotide sequence encoding the anti-BCMA CAR (e.g., SEQ ID NO: 33; see Table 4 below) may be inserted into the TRAC gene locus. The CAR-coding sequence is in operably linkage to a EF-1a promoter such as SEQ ID NO: 38. A poly A sequence (e.g., SEQ ID NO:39) can be located downstream of the coding sequence. See Table 4 below.

Further, a portion of the genetically engineered anti-BCMA CAR-T cells (e.g., CTX120 cells) comprise a plurality of disrupted β2M genes, which collectively may comprise one or more of the edited β2M gene sequence listed in Table 3 below (“-” indicates deletion and residues in boldface indicate mutation or insertion):

TABLE 3
Edited β2M Gene Sequence

| | Sequence (Deletions indicated by dashes (-); | |
|---|---|---|
| Description | insertions indicated by bold) | SEQ ID NO: |
| β2M gene-edit | CGTGGCCTTAGCTGTGCTCGCGCTACTCTCTCTTTCT- | 21 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTTC-- | 22 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTT----- | 23 |
| | CTGGAGGCTATCCAGCGTGAGTCTCTCCTACCCTCCCGCT | |
| β2M gene-edit | CGTGGCCTTAGCTGTGCTCGCGCTACTCTCTCTTTCTGGATA | 24 |
| | GCCTGGAGGCTATCCAGCGTGAGTCTCTCCTACCCTCCCGCT | |
| β2M gene-edit | CGTGGCCTTAGCTGTGCTCGC------------------------- | 25 |
| | GCTATCCAGCGTGAGTCTCTCCTACCCTCCCGCT | |
| β2M gene-edit | CGTGGCCTTAGCTGTGCTCGCGCTACTCTCTCTTTCTGTGGC | 26 |
| | CTGGAGGCTATCCAGCGTGAGTCTCTCCTACCCTCCCGCT | |

The components of the rAAV encoding the anti-BCMA CAR, including nucleotide sequences and amino acid sequences are provided in Table 4 and Table 5, respectively.

TABLE 4
Gene Editing/CAR Construct Components (Nucleotide Sequences)

| Name | | SEQ ID |
|---|---|---|
| Description | Nucleotide Sequence | NO: |
| CTX-166b | CCTGCAGGCAGCTGCGCGCTCGCTCGCTCACTGAGGCCGCCCGGGCGTCGGGCGAC | 27 |
| rAAV | CTTTGGTCGCCCGGCCTCAGTGAGCGAGCGAGCGCGCAGAGAGGGAGTGGCCAAC | |
| | TCCATCACTAGGGGTTCCTGCGGCCGCACGCGTGAGATGTAAGGAGCTGCTGTGAC | |
| | TTGCTCAAGGCCTTATATCGAGTAAACGGTAGTGCTGGGGCTTAGACGCAGGTGTT | |
| | CTGATTTATAGTTCAAAACCTCTATCAATGAGAGAGCAATCTCCTGGTAATGTGAT | |
| | AGATTTCCCAACTTAATGCCAACATACCATAAACCTCCCATTCTGCTAATGCCCAG [symbol] | |
| | CTAAGTTGGGGAGACCACTCCAGATTCCAAGATGTACAGTTTGCTTTGCTGGGCCT | |
| | TTTCCCATGCCTGCCTTTACTCTGCCAGAGTTATATTGCTGGGGTTTTGAAGAAGAT | |
| | CCTATTAAATAAAAGAATAAGCAGTATTATTAAGTAGCCCTGCATTTCAGGTTTCC [symbol] | |
| | TGAGTGGCAGGCCAGGCCTGGCCGTGAACGTTCACTGAAATCATGGCCTCTTGGCC | |
| | AAGATTGATAGCTTGTGCCTGTCCCTGAGTCCCAGTCCATCACGAGCAGCTGGTTT [symbol] | |
| | TAAGATGCTATTTCCCGTATAAAGCATGAGACCGTGACTTGCCAGCCCCACAGAGC | |
| | CCCGCCCTTGTCCATCACTGGCATCTGGACTCCAGCCTGGGTTGGGGCAAAGAGGG | |
| | AAATGAGATCATGTCCTAACCCTGATCCTCTTGTCCCACAGATATCCAGAACCCTG | |
| | ACCCTGCCGTGTACCAGCTGAGAGACTCTAAATCCAGTGACAAGTCTGTCTGCCTA | |
| | TTCACCGATTTTGATTCTCAAACAAATGTGTCACAAAGTAAGGATTCTGATGTGTA [symbol] | |
| | ATCACAGACAAAACTGTGCTAGACATGAGGTCTATGGACTTCAGGCTCCGGTGCCC | |
| | GTCAGTGGGCAGAGCGCACATCGCCCACAGTCCCCGAGAAGTTGGGGGGAGGGGT | |
| | CGGCAATTGAACCGGTGCCTAGAGAAGGTGGCGCGGGGTAAACTGGGAAAGTGAT | |
| | GTCGTGTACTGGCTCCGCCTTTTTCCCGAGGGTGGGGGAGAACCGTATATAAGTGC | |
| | AGTAGTCGCCGTGAACGTTCTTTTTCGCAACGGGTTTGCCGCCAGAACACAGGTAA | |
| | GTGCCGTGTGTGGTTCCCGCGGGCCTGGCCTCTTTACGGGTTATGGCCCTTGCGTGC | |
| | CTTGAATTACTTCCACTGGCTGCAGTACGTGATTCTTGATCCCGAGCTTCGGGTTGG | |
| | AAGTGGGTGGGAGAGTTCGAGGCCTTGCGCTTAAGGAGCCCCTTCGCCTCGTGCTT | |
| | GAGTTGAGGCCTGGCCTGGGCGCTGGGGCCGCCGCGTGCGAATCTGGTGGCACCT | |
| | CGCGCCTGTCTCGCTGCTTTCGATAAGTCTCTAGCCATTTAAAATTTTTGATGACCT | |
| | GCTGCGACGCTTTTTTTCTGGCAAGATAGTCTTGTAAATGCGGGCCAAGATCTGCA [symbol] | |
| | ACTGGTATTTCGGTTTTTGGGGCCGCGGGCGGCGACGGGGCCCGTGCGTCCCAGCG | |
| | CACATGTTCGGCGAGGCGGGGCCTGCGAGCGCGGCCACCGAGAATCGGACGGGGG | |
| | TAGTCTCAAGCTGGCCGGCCTGCTCTGGTGCCTGGCCTCGCGCCGCCGTGTATCGC [symbol] | |
| | CCGCCCTGGGCGGCAAGGCTGGCCCGGTCGGCACCAGTTGCGTGAGCGGAAAGAT | |
| | GGCCGCTTCCCGGCCCTGCTGCAGGGAGCTCAAAATGGAGGACGCGGCGCTCGGG | |
| | AGAGCGGGCGGGTGAGTCACCCACACAAAGGAAAAGGGCCTTTCCGTCCTCAGCC | |
| | GTCGCTTCATGTGACTCCACGGAGTACCGGGCGCCGTCCAGGCACCTCGATTAGTT | |
| | CTCGAGCTTTTGGAGTACGTCGTCTTTAGGTTGGGGGGAGGGGTTTTATGCGATGG | |
| | AGTTTCCCCACACTGAGTGGGTGGAGACTGAAGTTAGGCCAGCTTGGCACTTGATG | |
| | TAATTCTCCTTGGAATTTGCCCTTTTTGAGTTTGGATCTTGGTTCATTCTCAAGCCTC | |
| | AGACAGTGGTTCAAAGTTTTTTTCTTCCATTTCAGGTGTCGTGACCACCATGGCGCT | |
| | TCCGGTGACAGCACTGCTCCTCCCCTTGGCGCTGTTGCTCCACGCAGCAAGGCCGC | |
| | AGGTGCAGCTGGTGCAGAGCGGAGCCGAGCTCAAGAAGCCCGGAGCCTCCGTGAA | |
| | GGTGAGCTGCAAGGCCAGCGGCAACACCCTGACCAACTACGTGATCCACTGGGTG | |
| | AGACAAGCCCCCGGCCAAAGGCTGGAGTGGATGGGCTACATCCTGCCCTACAACG | |
| | ACCTGACCAAGTACAGCCAGAAGTTCCAGGGCAGGGTGACCATCACCAGGGATAA | |
| | GAGCGCCTCCACCGCCTATATGGAGCTGAGCAGCCTGAGGAGCGAGGACACCGCT | |
| | GTGTACTACTGTACAAGGTGGGACTGGGACGGCTTCTTTGACCCCTGGGGCCAGGG | |
| | CACAACAGTGACCGTCAGCAGCGGCGGCGGAGGCAGCGGCGGCGGCGGCAGCGG [symbol] | |
| | GGAGGCGGAAGCGAAATCGTGATGACCCAGAGCCCCGCCACACTGAGCGTGAGCC | |
| | CTGGCGAGAGGGCCAGCATCTCCTGCAGGGCTAGCCAAAGCCTGGTGCACAGCAA | |
| | CGGCAACACCCACCTGCACTGGTACCAGCAGAGACCCGGACAGGCTCCCAGGCTG | |
| | CTGATCTACAGCGTGAGCAACAGGTTCTCCGAGGTGCCTGCCAGGTTTAGCGGCAG | |
| | CGGAAGCGGCACCGACTTTACCCTGACCATCAGCAGCGTGGAGTCCGAGGACTTCC | |
| | CCGTGTATTACTGCAGCCAGACCAGCCACATCCCTTACACCTTCGGCGGCGGCACC | |
| | AAGCTGGAGATCAAAAGTGCTGCTGCCTTTGTCCCGGTATTTCTCCCAGCCAAACC | |
| | GACCACGACTCCCGCCCCGCGCCCTCCGACACCCGCTCCCACCATCGCCTCTCAAC | |
| | CTCTTAGTCTTCGCCCCGAGGCATGCCGACCCGCCGCCGGGGGTGCTGTTCATACG | |
| | AGGGGCTTGGACTTCGCTTGTGATATTTACATTTGGGCTCCGTTGGCGGGTACGTGC | |
| | GGCGTCCTTTTGTTGTCACTCGTTATTACTTTGTATTGTAATCACAGGAATCGCAAA | |
| | CGGGGCAGAAAGAAACTCCTGTATATATTCAAACAACCATTTATGAGACCAGTACA | |
| | AACTACTCAAGAGGAAGATGGCTGTAGCTGCCGATTTCCAGAAGAAGAAGAAGGA | |
| | GGATGTGAACTGCGAGTGAAGTTTTCCCGAAGCGCAGACGCTCCGGCATATCAGCA | |
| | AGGACAGAATCAGCTGTATAACGAACTGAATTTGGGACGCCGCGAGGAGTATGAC | |
| | GTGCTTGATAAACGCCGGGGGAGAGACCCGGAAATGGGGGGTAAACCCCGAAGAA | |
| | AGAATCCCCAAGAAGGACTCTACAATGAACTCCAGAAGGATAAGATGGCGGAGGC | |
| | CTACTCAGAAATAGGTATGAAGGGCGAACGACGACGGGGAAAAGGTCACGATGGC | |
| | CTCTACCAAGGGTTGAGTACGGCAACCAAAGATACGTACGATGCACTGCATATGCA | |
| | GGCCCTGCCTCCCAGATAATAATAAAATCGCTATCCATCGAAGATGGATGTGTGTT | |
| | GGTTTTTTGTGTGTGGAGCAACAAATCTGACTTTGCATGTGCAAACGCCTTCAACAA | |
| | CAGCATTATTCCAGAAGACACCTTCTTCCCCAGCCCAGGTAAGGGCAGCTTTGGTG | |
| | CCTTCGCAGGCTGTTTCCTTGCTTCAGGAATGGCCAGGTTCTGCCCAGAGCTCTGGT | |
| | CAATGATGTCTAAAACTCCTCTGATTGGTGGTCTCGGCCTTATCCATTGCCACCAAA | |
| | ACCCTCTTTTTACTAAGAAACAGTGAGCCTTGTTCTGGCAGTCCAGAGAATGACAC | |
| | GGGAAAAAAGCAGATGAAGAGAAGGTGGCAGGAGAGGGCACGTGGCCCAGCCTC | |
| | AGTCTCTCCAACTGAGTTCCTGCCTGCCTGCCTTTGCTCAGACTGTTTGCCCCTTAC [symbol] | |
| | GCTCTTCTAGGCCTCATTCTAAGCCCCTTCTCCAAGTTGCCTCTCCTTATTTCTCCCT | |
| | GTCTGCCAAAAAATCTTTCCCAGCTCACTAAGTCAGTCTCACGCAGTCACTCATTAA | |
| | CCCACCAATCACTGATTGTGCCGGCACATGAATGCACCAGGTGTTGAAGTGGAGGA | |
| | ATTAAAAAGTCAGATGAGGGGTGTGCCCAGAGGAAGCACCATTCTAGTTGGGGGA | |
| | GCCCATCTGTCAGCTGGGAAAAGTCCAAATAACTTCAGATTGGAATGTGTTTTAAC | |
| | TCAGGGTTGAGAAAACAGCTACCTTCAGGACAAAAGTCAGGGAAGGGCTCTCTGA | |
| | AGAAATGCTACTTGAAGATACCAGCCCTACCAAGGGCAGGGAGAGGACCCTATAG | |
| | AGGCCTGGGACAGGAGCTCAATGAGAAAGGTAACCACGTGCGGACCGAGGCTGCA | |
| | GCGTCGTCCTCCCTAGGAACCCCTAGTGATGGAGTTGGCCACTCCCTCTCTGCGCG [symbol] | |
| | TCGCTCGCTCACTGAGGCCGGGCGACCAAAGGTCGCCCGACGCCCGGGCTTTGCCC | |
| | GGGCGGCCTCAGTGAGCGAGCGAGCGCGCAGCTGCCTGCAGG | |
| 5′ITR | CCTGCAGGCAGCTGCGCGCTCGCTCGCTCACTGAGGCCGCCCGGGCGTCGGGCGA [symbol] | 28 |
| | CTTTGGTCGCCCGGCCTCAGTGAGCGAGCGAGCGCGCAGAGAGGGAGTGGCCAAC | |
| | TCCATCACTAGGGGTTCCT | |
| 3′ITR | AGGAACCCCTAGTGATGGAGTTGGCCACTCCCTCTCTGCGCGCTCGCTCGCTCAC [symbol] | 29 |
| | AGGCCGGGCGACCAAAGGTCGCCCGACGCCCGGGCTTTGCCCGGGCGGCCTCAGT | |
| | GAGCGAGCGAGCGCGCAGCTGCCTGCAGG | |
| LHA to RHA | GAGATGTAAGGAGCTGCTGTGACTTGCTCAAGGCCTTATATCGAGTAAACGGTAGT | 30 |
| (CTX-166b) | GCTGGGGCTTAGACGCAGGTGTTCTGATTTATAGTTCAAAACCTCTATCAATGAGA | |
| | GAGCAATCTCCTGGTAATGTGATAGATTTCCCAACTTAATGCCAACATACCATAAA | |
| | CCTCCCATTCTGCTAATGCCCAGCCTAAGTTGGGGAGACCACTCCAGATTCCAAGA | |
| | TGTACAGTTTGCTTTGCTGGGCCTTTTTCCCATGCCTGCCTTTACTCTGCCAGAGTTA | |
| | TATTGCTGGGGTTTTGAAGAAGATCCTATTAAATAAAAGAATAAGCAGTATTATTA | |
| | AGTAGCCCTGCATTTCAGGTTTCCTTGAGTGGCAGGCCAGGCCTGGCCGTGAACGT | |
| | TCACTGAAATCATGGCCTCTTGGCCAAGATTGATAGCTTGTGCCTGTCCCTGAGTC [symbol] | |
| | CAGTCCATCACGAGCAGCTGGTTTCTAAGATGCTATTTCCCGTATAAAGCATGAGA | |
| | CCGTGACTTGCCAGCCCCACAGAGCCCCGCCCTTGTCCATCACTGGCATCTGGACT [symbol] | |
| | CAGCCTGGGTTGGGGCAAAGAGGGAAATGAGATCATGTCCTAACCCTGATCCTCTT | |
| | GTCCCACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATGTGTCA | |
| | CAAAGTAAGGATTCTGATGTGTATATCACAGACAAAACTGTGCTAGACATGAGGT [symbol] | |
| | TATGGACTTCAGGCTCCGGTGCCCGTCAGTGGGCAGAGCGCACATCGCCCACAGT [symbol] | |
| | CCCGAGAAGTTGGGGGGAGGGGTCGGCAATTGAACCGGTGCCTAGAGAAGGTGGC | |
| | GCGGGGTAAACTGGGAAAGTGATGTCGTGTACTGGCTCCGCCTTTTTCCCGAGGGT | |
| | GGGGGAGAACCGTATATAAGTGCAGTAGTCGCCGTGAACGTTCTTTTTCGCAACGG | |
| | GTTTGCCGCCAGAACACAGGTAAGTGCCGTGTGTGGTTCCCGCGGGCCTGGCCTCT | |
| | TTACGGGTTATGGCCCTTGCGTGCCTTGAATTACTTCCACTGGCTGCAGTACGTGAT | |
| | TCTTGATCCCGAGCTTCGGGTTGGAAGTGGGTGGGAGAGTTCGAGGCCTTGCGCTT | |
| | AAGGAGCCCCTTCGCCTCGTGCTTGAGTTGAGGCCTGGCCTGGGCGCTGGGGCCGC | |
| | CGCGTGCGAATCTGGTGGCACCTTCGCGCCTGTCTCGCTGCTTTCGATAAGTCTCTA | |
| | GCCATTTAAAATTTTTGATGACCTGCTGCGACGCTTTTTTTCTGGCAAGATAGTCTT | |
| | GTAAATGCGGGCCAAGATCTGCACACTGGTATTTCGGTTTTTGGGGCCGCGGGCGG | |
| | CGACGGGGCCCGTGCGTCCCAGCGCACATGTTCGGCGAGGCGGGGCCTGCGAGCG | |
| | CGGCCACCGAGAATCGGACGGGGGTAGTCTCAAGCTGGCCGGCCTGCTCTGGTGC [symbol] | |
| | TGGCCTCGCGCCGCCGTGTATCGCCCCGCCCTGGGCGGCAAGGCTGGCCCGGTCGG | |
| | CACCAGTTGCGTGAGCGGAAAGATGGCCGCTTCCCGGCCCTGCTGCAGGGAGCTCA | |
| | AAATGGAGGACGCGGCGCTCGGGAGAGCGGGCGGGTGAGTCACCCACACAAAGGA | |
| | AAAGGGCCTTTCCGTCCTCAGCCGTCGCTTCATGTGACTCCACGGAGTACCGGGCG | |
| | CCGTCCAGGCACCTCGATTAGTTCTCGAGCTTTTGGAGTACGTCGTCTTTAGGTTGG | |
| | GGGGAGGGGTTTTATGCGATGGAGTTTCCCCACACTGAGTGGGTGGAGACTGAAGT | |
| | TAGGCCAGCTTGGCACTTGATGTAATTCTCCTTGGAATTTGCCCTTTTTGAGTTTGG | |
| | ATCTTGGTTCATTCTCAAGCCTCAGACAGTGGTTCAAAGTTTTTTTCTTCCATTTCA [symbol] | |
| | GTGTCGTGACCACCATGGCGCTTCCGGTGACAGCACTGCTCCTCCCCTTGGCGCTG [symbol] | |
| | TGCTCCACGCAGCAAGGCCGCAGGTGCAGCTGGTGCAGAGCGGAGCCGAGCTCAA | |
| | GAAGCCCGGAGCCTCCGTGAAGGTGAGCTGCAAGGCCAGCGGCAACACCCTGACC | |
| | AACTACGTGATCCACTGGGTGAGACAAGCCCCCGGCCAAAGGCTGGAGTGGATGG | |
| | GCTACATCCTGCCCTACAACGACCTGACCAAGTACAGCCAGAAGTTCCAGGGCAG [symbol] | |
| | GTGACCATCACCAGGGATAAGAGCGCCTCCACCGCCTATATGGAGCTGAGCAGCCT | |
| | GAGGAGCGAGGACACCGCTGTGTACTACTGTACAAGGTGGGACTGGGACGGCTTCT | |
| | TTGACCCCTGGGGCCAGGGCACAACAGTGACCGTCAGCAGCGGCGGCGGAGGCAG | |
| | CGGCGGCGGCGGCAGCGGCGGAGGCGGAAGCGAAATCGTGATGACCCAGAGCCCC | |
| | GCCACACTGAGCGTGAGCCCTGGCGAGAGGGCCAGCATCTCCTGCAGGGCTAGCC | |
| | AAAGCCTGGTGCACAGCAACGGCAACACCCACCTGCACTGGTACCAGCAGAGACC | |
| | CGGACAGGCTCCCAGGCTGCTGATCTACAGCGTGAGCAACAGGTTCTCCGAGGTG [symbol] | |
| | CTGCCAGGTTTAGCGGCAGCGGAAGCGGCACCGACTTTACCCTGACCATCAGCAG [symbol] | |
| | GTGGAGTCCGAGGACTTCGCCGTGTATTACTGCAGCCAGACCAGCCACATCCCTTA | |
| | CACCTTCGGCGGCGGCACCAAGCTGGAGATCAAAAGTGCTGCTGCCTTTGTCCCGG | |
| | TATTTCTCCCAGCCAAACCGACCACGACTCCCGCCCCGCGCCCTCCGACACCCGCT [symbol] | |
| | CCACCATCGCCTCTCAACCTCTTAGTCTTCGCCCCGAGGCATGCCGACCCGCCGCC [symbol] | |
| | GGGGTGCTGTTCATACGAGGGGCTTGGACTTCGCTTGTGATATTTACATTTGGGCT [symbol] | |
| | CGTTGGCGGGTACGTGCGGCGTCCTTTTGTTGTCACTCGTTATTACTTTGTATTGTA | |
| | ATCACAGGAATCGCAAACGGGGCAGAAAGAAACTCCTGTATATATTCAAACAACC | |
| | ATTTATGAGACCAGTACAAACTACTCAAGAGGAAGATGGCTGTAGCTGCCGATTTC | |
| | CAGAAGAAGAAGAAGGAGGATGTGAACTGCGAGTGAAGTTTTCCCGAAGCGCAGA | |
| | CGCTCCGGCATATCAGCAAGGACAGAATCAGCTGTATAACGAACTGAATTTGGGA [symbol] | |
| | GCCGCGAGGAGTATGACGTGCTTGATAAACGCCGGGGGAGAGACCCGGAAATGGG | |
| | GGGTAAACCCCGAAGAAAGAATCCCCAAGAAGGACTCTACAATGAACTCCAGAAG | |
| | GATAAGATGGCGGAGGCCTACTCAGAAATAGGTATGAAGGGCGAACGACGACGG [symbol] | |
| | GAAAAGGTCACGATGGCCTCTACCAAGGGTTGAGTACGGCAACCAAAGATACGTA | |
| | CGATGCACTGCATATGCAGGCCCTGCCTCCCAGATAATAATAAAATCGCTATCCAT | |
| | CGAAGATGGATGTGTGTTGGTTTTTTGTGTGTGGAGCAACAAATCTGACTTTGCAT [symbol] | |
| | TGCAAACGCCTTCAACAACAGCATTATTCCAGAAGACACCTTCTTCCCCAGCCCAG | |
| | GTAAGGGCAGCTTTGGTGCCTTCGCAGGCTGTTTCCTTGCTTCAGGAATGGCCAGG [symbol] | |
| | TCTGCCCAGAGCTCTGGTCAATGATGTCTAAAACTCCTCTGATTGGTGGTCTCGGC [symbol] | |
| | TTATCCATTGCCACCAAAACCCTCTTTTTACTAAGAAACAGTGAGCCTTGTTCTGGC | |
| | AGTCCAGAGAATGACACGGGAAAAAAGCAGATGAAGAGAAGGTGGCAGGAGAGG | |
| | GCACGTGGCCCAGCCTCAGTCTCTCCAACTGAGTTCCTGCCTGCCTGCCTTTGCTCA | |
| | GACTGTTTGCCCCTTACTGCTCTTCTAGGCCTCATTCTAAGCCCCTTCTCCAAGTTG [symbol] | |
| | CTCTCCTTATTTCTCCCTGTCTGCCAAAAAATCTTTCCCAGCTCACTAAGTCAGTCT [symbol] | |
| | ACGCAGTCACTCATTAACCCACCAATCACTGATTGTGCCGGCACATGAATGCACCA | |
| | GGTGTTGAAGTGGAGGAATTAAAAAGTCAGATGAGGGGTGTGCCCAGAGGAAGCA | |
| | CCATTCTAGTTGGGGGAGCCCATCTGTCAGCTGGGAAAAGTCCAAATAACTTCAGA | |
| | TTGGAATGTGTTTTAACTCAGGGTTGAGAAAACAGCTACCTTCAGGACAAAAGTCA | |
| | GGGAAGGGCTCTCTGAAGAAATGCTACTTGAAGATACCAGCCCTACCAAGGGCAG | |
| | GGAGAGGACCCTATAGAGGCCTGGGACAGGAGCTCAATGAGAAAGG | |
| TRAC-LHA | GAGATGTAAGGAGCTGCTGTGACTTGCTCAAGGCCTTATATCGAGTAAACGGTAGT | 31 |
| (800 bp) | GCTGGGGCTTAGACGCAGGTGTTCTGATTTATAGTTCAAAACCTCTATCAATGAGA | |
| | GAGCAATCTCCTGGTAATGTGATAGATTTCCCAACTTAATGCCAACATACCATAAA | |
| | CCTCCCATTCTGCTAATGCCCAGCCTAAGTTGGGGAGACCACTCCAGATTCCAAGA | |
| | TGTACAGTTTGCTTTGCTGGGCCTTTTTCCCATGCCTGCCTTTACTCTGCCAGAGTTA | |
| | TATTGCTGGGGTTTTGAAGAAGATCCTATTAAATAAAAGAATAAGCAGTATTATTA | |
| | AGTAGCCCTGCATTTCAGGTTTCCTTGAGTGGCAGGCCAGGCCTGGCCGTGAACGT | |
| | TCACTGAAATCATGGCCTCTTGGCCAAGATTGATAGCTTGTGCCTGTCCCTGAGTCC | |
| | CAGTCCATCACGAGCAGCTGGTTTCTAAGATGCTATTTCCCGTATAAAGCATGAGA | |
| | CCGTGACTTGCCAGCCCCACAGAGCCCCGCCCTTGTCCATCACTGGCATCTGGACT [symbol] | |
| | CAGCCTGGGTTGGGGCAAAGAGGGAAATGAGATCATGTCCTAACCCTGATCCTCTT | |
| | GTCCCACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATGTGTCA | |
| | CAAAGTAAGGATTCTGATGTGTATATCACAGACAAAACTGTGCTAGACATGAGGT [symbol] | |
| | TATGGACTTCA | |
| TRAC-RHA | TGGAGCAACAAATCTGACTTTGCATGTGCAAACGCCTTCAACAACAGCATTATTCC | 32 |
| (800 bp) | AGAAGACACCTTCTTCCCCAGCCCAGGTAAGGGCAGCTTTGGTGCCTTCGCAGGCT | |
| | GTTTCCTTGCTTCAGGAATGGCCAGGTTCTGCCCAGAGCTCTGGTCAATGATGTCT | |
| | AAACTCCTCTGATTGGTGGTCTCGGCCTTATCCATTGCCACCAAAACCCTCTTTTTA | |
| | CTAAGAAACAGTGAGCCTTGTTCTGGCAGTCCAGAGAATGACACGGGAAAAAAGC | |
| | AGATGAAGAGAAGGTGGCAGGAGAGGGCACGTGGCCCAGCCTCAGTCTCTCCAAC | |
| | TGAGTTCCTGCCTGCCTGCCTTTGCTCAGACTGTTTGCCCCTTACTGCTCTTCTAGG [symbol] | |
| | CTCATTCTAAGCCCCTTCTCCAAGTTGCCTCTCCTTATTTCTCCCTGTCTGCCAAAAA | |
| | ATCTTTCCCAGCTCACTAAGTCAGTCTCACGCAGTCACTCATTAACCCACCAATCA [symbol] | |
| | TGATTGTGCCGGCACATGAATGCACCAGGTGTTGAAGTGGAGGAATTAAAAAGTC [symbol] | |
| | GATGAGGGGTGTGCCCAGAGGAAGCACCATTCTAGTTGGGGGAGCCCATCTGTCA [symbol] | |
| | CTGGGAAAAGTCCAAATAACTTCAGATTGGAATGTGTTTTAACTCAGGGTTGAGAA | |
| | AACAGCTACCTTCAGGACAAAAGTCAGGGAAGGGCTCTCTGAAGAAATGCTACTT [symbol] | |
| | AAGATACCAGCCCTACCAAGGGCAGGGAGAGGACCCTATAGAGGCCTGGGACAG [symbol] | |
| | AGCTCAATGAGAAAGG | |
| Anti-BCMA | ATGGCGCTTCCGGTGACAGCACTGCTCCTCCCCTTGGCGCTGTTGCTCCACGCAGCA | 33 |
| CAR | AGGCCGCAGGTGCAGCTGGTGCAGAGCGGAGCCGAGCTCAAGAAGCCCGGAGCCT | |
| (CTX-166b) | CCGTGAAGGTGAGCTGCAAGGCCAGCGGCAACACCCTGACCAACTACGTGATCCA | |
| | CTGGGTGAGACAAGCCCCCGGCCAAAGGCTGGAGTGGATGGGCTACATCCTGCCCT | |
| | ACAACGACCTGACCAAGTACAGCCAGAAGTTCCAGGGCAGGGTGACCATCACCAG | |
| | GGATAAGAGCGCCTCCACCGCCTATATGGAGCTGAGCAGCCTGAGGAGCGAGGAC | |
| | ACCGCTGTGTACTACTGTACAAGGTGGGACTGGGACGGCTTCTTTGACCCCTGGGG | |
| | CCAGGGCACAACAGTGACCGTCAGCAGCGGCGGCGGAGGCAGCGGCGGCGGCGG [symbol] | |
| | AGCGGCGGAGGCGGAAGCGAAATCGTGATGACCCAGAGCCCCGCCACACTGAGCG | |
| | TGAGCCCTGGCGAGAGGGCCAGCATCTCCTGCAGGGCTAGCCAAAGCCTGGTGCA [symbol] | |
| | AGCAACGGCAACACCCACCTGCACTGGTACCAGCAGAGACCCGGACAGGCTCCCA | |
| | GGCTGCTGATCTACAGCGTGAGCAACAGGTTCTCCGAGGTGCCTGCCAGGTTTAGC | |
| | GGCAGCGGAAGCGGCACCGACTTTACCCTGACCATCAGCAGCGTGGAGTCCGAGG | |
| | ACTTCGCCGTGTATTACTGCAGCCAGACCAGCCACATCCCTTACACCTTCGGCGGC | |
| | GGCACCAAGCTGGAGATCAAAAGTGCTGCTGCCTTTGTCCCGGTATTTCTCCCAGC | |
| | CAAACCGACCACGACTCCCGCCCCGCGCCCTCCGACACCCGCTCCCACCATCGCCT | |
| | CTCAACCTCTTAGTCTTCGCCCCGAGGCATGCCGACCCGCCGCCGGGGGTGCTGTT [symbol] | |
| | ATACGAGGGGCTTGGACTTCGCTTGTGATATTTACATTTGGGCTCCGTTGGCGGGTA | |
| | CGTGCGGCGTCCTTTTGTTGTCACTCGTTATTACTTTGTATTGTAATCACAGGAATC | |
| | GCAAACGGGGCAGAAAGAAACTCCTGTATATATTCAAACAACCATTTATGAGACCA | |
| | GTACAAACTACTCAAGAGGAAGATGGCTGTAGCTGCCGATTTCCAGAAGAAGAAG | |
| | AAGGAGGATGTGAACTGCGAGTGAAGTTTTCCCGAAGCGCAGACGCTCCGGCATA [symbol] | |
| | CAGCAAGGACAGAATCAGCTGTATAACGAACTGAATTTGGGACGCCGCGAGGAGT | |
| | ATGACGTGCTTGATAAACGCCGGGGGAGAGACCCGGAAATGGGGGGTAAACCCCG | |
| | AAGAAAGAATCCCCAAGAAGGACTCTACAATGAACTCCAGAAGGATAAGATGGCG | |
| | GAGGCCTACTCAGAAATAGGTATGAAGGGCGAACGACGACGGGGAAAAGGTCAC [symbol] | |
| | ATGGCCTCTACCAAGGGTTGAGTACGGCAACCAAAGATACGTACGATGCACTGCAT | |
| | ATGCAGGCCCTGCCTCCCAGA | |
| Anti-BCMA | CAGGTGCAGCTGGTGCAGAGCGGAGCCGAGCTCAAGAAGCCCGGAGCCTCCGTGA | 34 |
| scFv | AGGTGAGCTGCAAGGCCAGCGGCAACACCCTGACCAACTACGTGATCCACTGGGT | |
| (CTX-166 & | GAGACAAGCCCCCGGCCAAAGGCTGGAGTGGATGGGCTACATCCTGCCCTACAAC | |
| CTX-166b) | GACCTGACCAAGTACAGCCAGAAGTTCCAGGGCAGGGTGACCATCACCAGGGATA | |
| | AGAGCGCCTCCACCGCCTATATGGAGCTGAGCAGCCTGAGGAGCGAGGACACCGC | |
| | TGTGTACTACTGTACAAGGTGGGACTGGGACGGCTTCTTTGACCCCTGGGGCCAGG | |
| | GCACAACAGTGACCGTCAGCAGCGGCGGCGGAGGCAGCGGCGGCGGCGGCAGCG [symbol] | |
| | CGGAGGCGGAAGCGAAATCGTGATGACCCAGAGCCCCGCCACACTGAGCGTGAGC | |
| | CCTGGCGAGAGGGCCAGCATCTCCTGCAGGGCTAGCCAAAGCCTGGTGCACAGCA | |
| | ACGGCAACACCCACCTGCACTGGTACCAGCAGAGACCCGGACAGGCTCCCAGGCT | |
| | GCTGATCTACAGCGTGAGCAACAGGTTCTCCGAGGTGCCTGCCAGGTTTAGCGGCA | |
| | GCGGAAGCGGCACCGACTTTACCCTGACCATCAGCAGCGTGGAGTCCGAGGACTTC | |
| | GCCGTGTATTACTGCAGCCAGACCAGCCACATCCCTTACACCTTCGGCGGCGGCAC | |
| | CAAGCTGGAGATCAAA | |
| 4-1BB | AAACGGGGCAGAAAGAAACTCCTGTATATATTCAAACAACCATTTATGAGACCAGT | 35 |
| | ACAAACTACTCAAGAGGAAGATGGCTGTAGCTGCCGATTTCCAGAAGAAGAAGAA | |
| | GGAGGATGTGAACTG | |
| CD28 | TCAAAGCGGAGTAGGTTGTTGCATTCCGATTACATGAATATGACTCCTCGCCGGCC | 36 |
| | TGGGCCGACAAGAAAACATTACCAACCCTATGCCCCCCCACGAGACTTCGCTGCGT | |
| | ACAGGTCC | |
| CD3-zeta | CGAGTGAAGTTTTCCCGAAGCGCAGACGCTCCGGCATATCAGCAAGGACAGAATC | 37 |
| | AGCTGTATAACGAACTGAATTTGGGACGCCGCGAGGAGTATGACGTGCTTGATAAA | |
| | CGCCGGGGGAGAGACCCGGAAATGGGGGGTAAACCCCGAAGAAAGAATCCCCAA | |
| | GAAGGACTCTACAATGAACTCCAGAAGGATAAGATGGCGGAGGCCTACTCAGAAA | |
| | TAGGTATGAAGGGCGAACGACGACGGGGAAAAGGTCACGATGGCCTCTACCAAGG | |
| | GTTGAGTACGGCAACCAAAGATACGTACGATGCACTGCATATGCAGGCCCTGCCT [symbol] | |
| | CCAGA | |
| EF-1α | GGCTCCGGTGCCCGTCAGTGGGCAGAGCGCACATCGCCCACAGTCCCCGAGAAGTT | 38 |
| promoter | GGGGGGAGGGGTCGGCAATTGAACCGGTGCCTAGAGAAGGTGGCGCGGGGTAAA [symbol] | |
| | TGGGAAAGTGATGTCGTGTACTGGCTCCGCCTTTTTCCCGAGGGTGGGGGAGAACC | |
| | GTATATAAGTGCAGTAGTCGCCGTGAACGTTCTTTTTCGCAACGGGTTTGCCGCCA [symbol] | |
| | AACACAGGTAAGTGCCGTGTGTGGTTCCCGCGGGCCTGGCCTCTTTACGGGTTATG | |
| | GCCCTTGCGTGCCTTGAATTACTTCCACTGGCTGCAGTACGTGATTCTTGATCCCGA | |
| | GCTTCGGGTTGGAAGTGGGTGGGAGAGTTCGAGGCCTTGCGCTTAAGGAGCCCCTT | |
| | CGCCTCGTGCTTGAGTTGAGGCCTGGCCTGGGCGCTGGGGCCGCCGCGTGCGAATC | |
| | TGGTGGCACCTTCGCGCCTGTCTCGCTGCTTTCGATAAGTCTCTAGCCATTTAAAAT | |
| | TTTTGATGACCTGCTGCGACGCTTTTTTTCTGGCAAGATAGTCTTGTAAATGCGGGC | |
| | CAAGATCTGCACACTGGTATTTCGGTTTTTGGGGCCGCGGGCGGCGACGGGGCCCG | |
| | TGCGTCCCAGCGCACATGTTCGGCGAGGCGGGGCCTGCGAGCGCGGCCACCGAGA | |
| | ATCGGACGGGGGTAGTCTCAAGCTGGCCGGCCTGCTCTGGTGCCTGGCCTCGCGCC | |
| | GCCGTGTATCGCCCCGCCCTGGGCGGCAAGGCTGGCCCGGTCGGCACCAGTTGCGT | |
| | GAGCGGAAAGATGGCCGCTTCCCGGCCCTGCTGCAGGGAGCTCAAAATGGAGGAC | |
| | GCGGCGCTCGGGAGAGCGGGCGGGTGAGTCACCCACACAAAGGAAAAGGGCCTTT | |
| | CCGTCCTCAGCCGTCGCTTCATGTGACTCCACGGAGTACCGGGCGCCGTCCAGGCA | |
| | CCTCGATTAGTTCTCGAGCTTTTGGAGTACGTCGTCTTTAGGTTGGGGGGAGGGGTT | |
| | TTATGCGATGGAGTTTCCCCACACTGAGTGGGTGGAGACTGAAGTTAGGCCAGCTT | |
| | GGCACTTGATGTAATTCTCCTTGGAATTTGCCCTTTTTGAGTTTGGATCTTGGTTCAT | |
| | TCTCAAGCCTCAGACAGTGGTTCAAAGTTTTTTTCTTCCATTTCAGGTGTCGTGA | |
| 3′ poly A | AATAAAATCGCTATCCATCGAAGATGGATGTGTGTTGGTTTTTTGTGTG | 39 |

[symbol]

indicates data missing or illegible when filed

TABLE 5
Anti-BCMA CAR Construct Components (Amino acid sequences)

| Name | | SEQ ID |
|---|---|---|
| Description | Amino Acid Sequence | NO: |
| CAR (CTX-166b) | MALPVTALLLPLALLLHAARPQVQLVQSGAELKKPGASVKVSCKASGNTLT | 40 |
| | NYVIHWVRQAPGQRLEWMGYILPYNDLTKYSQKFQGRVTITRDKSASTAYM | |
| | ELSSLRSEDTAVYYCTRWDWDGFFDPWGQGTTVTVSSGGGGSGGGGSGGG | |
| | GSEIVMTQSPATLSVSPGERASISCRASQSLVHSNGNTHLHWYQQRPGQAPRL | |
| | LIYSVSNRFSEVPARFSGSGSGTDFTLTISSVESEDFAVYYCSQTSHIPYTFGGG | |
| | TKLEIKSAAAFVPVFLPAKPTTTPAPRPPTPAPTIASQPLSLRPEACRPAAGGA [symbol] | |
| | HTRGLDFACDIYIWAPLAGTCGVLLLSLVITLYCNHRNRKRGRKKLLYIFKQ [symbol] | |
| | FMRPVQTTQEEDGCSCRFPEEEEGGCELRVKFSRSADAPAYQQGQNQLYNEL | |
| | NLGRREEYDVLDKRRGRDPEMGGKPRRKNPQEGLYNELQKDKMAEAYSEI [symbol] | |
| | MKGERRRGKGHDGLYQGLSTATKDTYDALHMQALPPR | |
| scFv | QVQLVQSGAELKKPGASVKVSCKASGNTLTNYVIHWVRQAPGQRLEWMGY | 41 |
| (CTX-166 (BCMA-11, | ILPYNDLTKYSQKFQGRVTITRDKSASTAYMELSSLRSEDTAVYYCTRWDWD | |
| & CTX-166b) | GFFDPWGQGTTVTVSSGGGGSGGGGSGGGGSEIVMTQSPATLSVSPGERASIS | |
| | CRASQSLVHSNGNTHLHWYQQRPGQAPRLLIYSVSNRFSEVPARFSGSGSGT | |
| | DFTLTISSVESEDFAVYYCSQTSHIPYTFGGGTKLEIK | |
| V_{H} (CTX-166) | QVQLVQSGAELKKPGASVKVSCKASGNTLTNYVIHWVRQAPGQRLEWMGY | 42 |
| | ILPYNDLTKYSQKFQGRVTITRDKSASTAYMELSSLRSEDTAVYYCTRWDWD | |
| | GFFDPWGQGTTVTVSS | |
| V_{L} (CTX-166) | EIVMTQSPATLSVSPGERASISCRASQSLVHSNGNTHLHWYQQRPGQAPRLLI | 43 |
| | YSVSNRFSEVPARFSGSGSGTDFTLTISSVESEDFAVYYCSQTSHIPYTFGGGT | |
| | KLEIK | |
| V_{L} CDR1 (Kabat or | RASQSLVHSNGNTHLH | |
| V_{L} CDR2 Chothia) | SVSNR | 45 |
| V_{L} CDR3 | SQTSHIPYT | 46 |
| V_{H} CDR1 (Kabat) | NYVIH | 47 |
| V_{H} CDR2 | YILPYNDLTKYSQKFQG | 48 |
| V_{H} CDR3 | WDWDGFFDP | 49 |
| V_{H} CDR1 (Chothia) | GNTLTNY | 50 |
| V_{H} CDR2 | LPYNDL | 51 |
| V_{H} CDR3 | WDWDGFFDP | 52 |
| linker | GGGGSGGGGSGGGGS | |
| Signal peptide-1 | MLLLVTSLLLCELPHPAFLLIP | 54 |
| CD8 signal peptide | MALPVTALLLPLALLLHAARP | 55 |
| CD8a transmembrane | IYIWAPLAGTCGVLLLSLVITLY | 56 |
| domain | | |
| 4-1BB | KRGRKKLLYIFKQPFMRPVQTTQEEDGCSCRFPEEEEGGCEL | 57 |
| CD28 | SKRSRLLHSDYMNMTPRRPGPTRKHYQPYAPPRDFAAYRS | 58 |
| CD3-zeta | RVKFSRSADAPAYQQGQNQLYNELNLGRREEYDVLDKRRGRDPEMGGKPR | 59 |
| | RKNPQEGLYNELQKDKMAEAYSEIGMKGERRRGKGHDGLYQGLSTATKDT | |
| | YDALHMQALPPR | |

[symbol]

indicates data missing or illegible when filed

At least a portion of the resultant genetically engineered anti-BCMA CAR-T cells (e.g., CTX120 cells) may comprise a disrupted TRAC gene, which has a deletion of at least the sequence of SEQ ID NO: 10, a disrupted β2M gene, and express an anti-BCMA CAR (e.g., SEQ ID NO: 40). Further, a portion of the cells in the CTX120 cell population may comprise a plurality of disrupted β2M genes, which collectively may comprise one or more of the sequences of SEQ ID NOs: 21-26. Further, the genetically engineered anti-BCMA CAR-T cells comprise the nucleotide sequence coding for the anti-BCMA CAR. In some examples, the CAR-coding sequence may be inserted into the TRAC gene locus (e.g., SEQ ID NO: 33, coding for the anti-BCMA CAR of SEQ ID NO:40). The anti-BCMA CAR coding sequence is in operable linkage to an EF-1a promoter, which may comprise the nucleotide sequence of SEQ ID NO: 38. Further, a poly A sequence (e.g., SEQ ID NO: 39) is located downstream of the coding sequence.

The resultant genetically engineered T cells were characterized for incorporation of the desired gene edits: loss of TCR, loss of MHC I expression, and expression of an anti-BCMA CAR. Approximately one week after gene-editing, allogeneic T cells were assessed for surface expression of TCR, β2M, and anti-BCMA CAR using flow cytometry. The allogeneic cells were stained with biotinylated recombinant human BCMA (Acro Biosystems Cat: #BC7-H82F0) and tagged with fluorescent streptavidin and with fluorescent antibodies targeting cell surface markers. The percentage of cells that were TCR⁻, β2M⁻, and anti-BCMA CAR⁺ was determined. Nine lots of CTX120 cells were prepared from eight healthy donors.

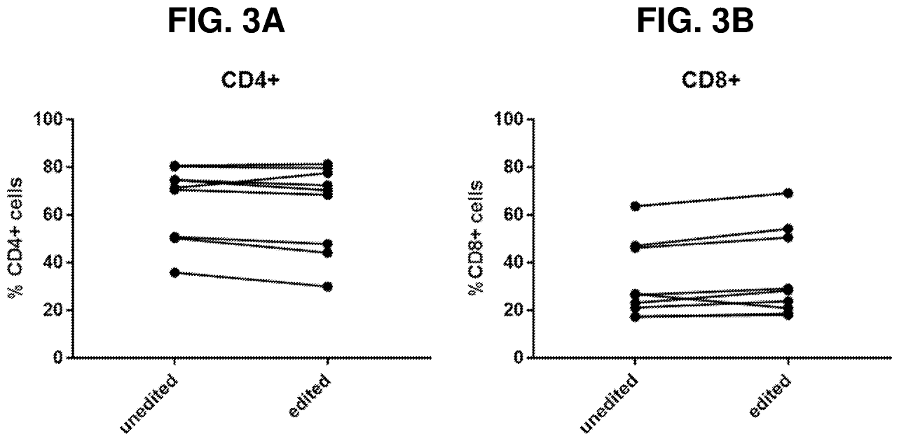

As shown in FIG. 2, reduction in TCR expression was nearly quantitative (96-99% of cells TCR⁻); reduction in β2M expression was also high (72-86% of cells β2M⁻); and anti-BCMA CAR incorporation ranged 46-79%. The percentage of CTX120 cells including the triple gene edits (TCR⁻, β2M⁻, and anti-BCMA CARP) was between 38% and 67%.

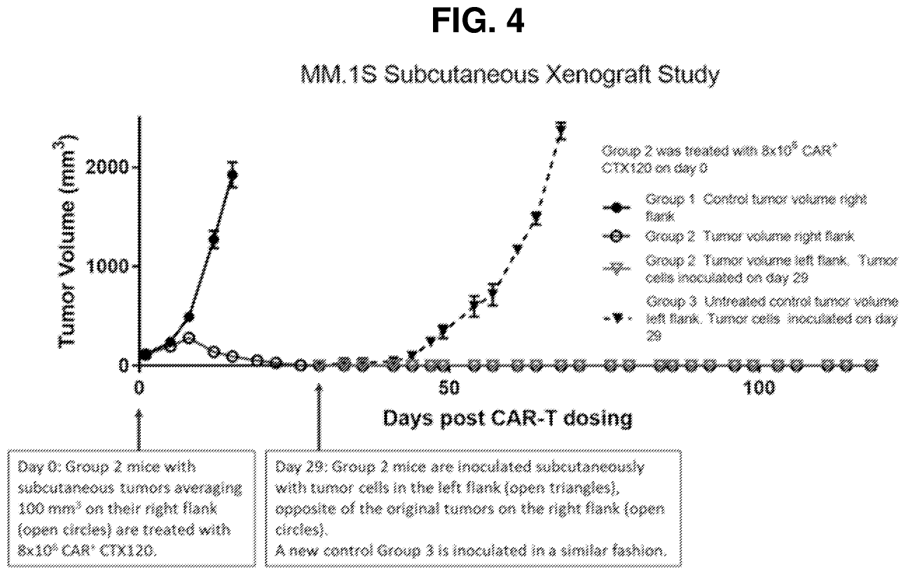

The percentage of the CTX120 cells that were CD4+ or CD8+ was also determined by flow cytometry. As shown in FIGS. 3A-3B, the percentage of CD4+ T cells (FIG. 3A) or CD8+ T cells (FIG. 3B) remained unchanged after the gene-editing process.

### Example 2: Anti-BCMA CAR T Cells Reduces Tumor Volume and Protects Against Re-Challenge in the MM.1S Tumor Model

The ability of CTX120 cells to limit growth of human BCMA-expressing MM tumors was evaluated in immunocompromised mice. The efficacy of CTX120 cells against the subcutaneous MM.1S tumor xenograft model in NOG mice (NOD.Cg-Prkdc^{scid}I12rg^{tm1Sug}/JicTac) was evaluated. In brief, 5 to 8-week old female NOG mice were individually housed in ventilated microisolator cages and maintained under pathogen-free conditions. The animals each received a subcutaneous inoculation in the right flank of 5×10⁶ MM.1S cells in 50% Matrigel. When the mean tumor volume reached 100 mm³ (approximately 75 to 125 mm³), the mice were randomized into two groups with 5 mice per group. One group was untreated, while the second group was dosed by intravenous injection of 8×10⁶ CTX120 CAR⁺ T cells.

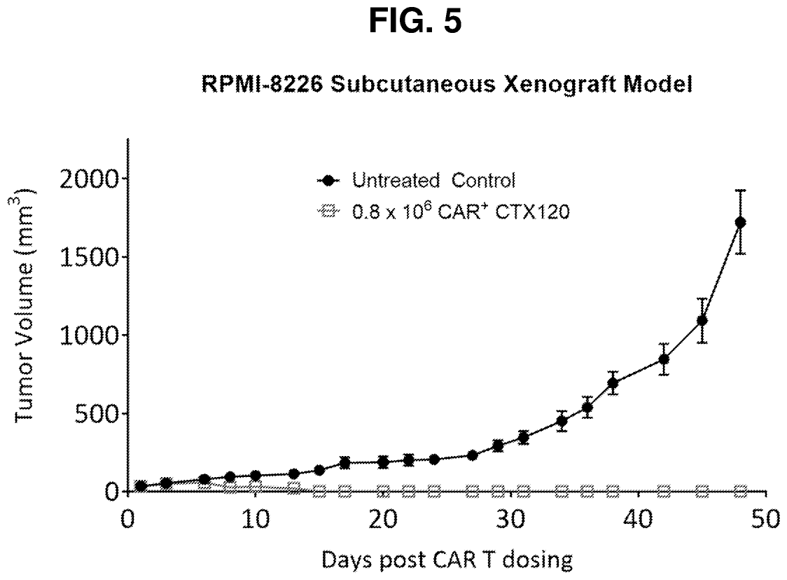

Tumor volume and body weights were measured twice weekly and individual mice were euthanized when their tumor volume reached ≥2000 mm³. By day 15, animals treated with CTX120 cells showed tumor regression from the starting volumes while animals in the control group had tumors averaging greater than 1000 mm³. By day 29, all animals in the control group had reached the tumor volume endpoint of ≥2000 mm³, whereas all treated animals had rejected the primary tumor burden (FIG. 4).

On day 29, all mice from the group receiving CTX120 treatment were further subjected to a second inoculation of MM.15 tumor cells (e.g., a tumor re-challenge). The mice received a second subcutaneous inoculation in the left flank of 5×10⁶ MM.1S cells in 50% Matrigel. Given that the first untreated group succumbed to tumor burden, a second cohort of tumor-free animals was administered the re-challenge inoculation in the left flank as a positive control.

All mice were monitored for tumor growth in both the initial right flank tumor and the re-challenge tumor in the left flank Animals treated with CTX120 cells successfully eliminated tumor growth in both the initial right flank tumor and in the re-challenge left flank tumor for the duration of the study, while untreated animals succumbed to tumor burden when given an inoculation of tumor cells in either the right or the left flank (FIG. 4).

### Example 3: Eradication of RPMI-8226 Tumors with Treatment of Anti-BCMA CAR T Cells

The efficacy of CTX120 was further evaluated in a second model of BCMA-expressing human MM, using the RPMI-8226 tumor xenograft model in NOG mice. In brief, 5 to 8-week old female, NOG (NOD.Cg-Prkdc^{scid}I12rg^{tm1Sug}/JicTac) mice were individually housed in ventilated microisolator cages and maintained under pathogen-free conditions. At 10 days prior to treatment, the mice received a subcutaneous inoculation of 10×10⁶ RPMI-8226 cells/mouse in the right flank. On day 1, the mice were randomized into groups (n=5 mice per group) and were either untreated or dosed with an intravenous injection of 0.8×10⁶ CAR-expressing CTX120 cells.

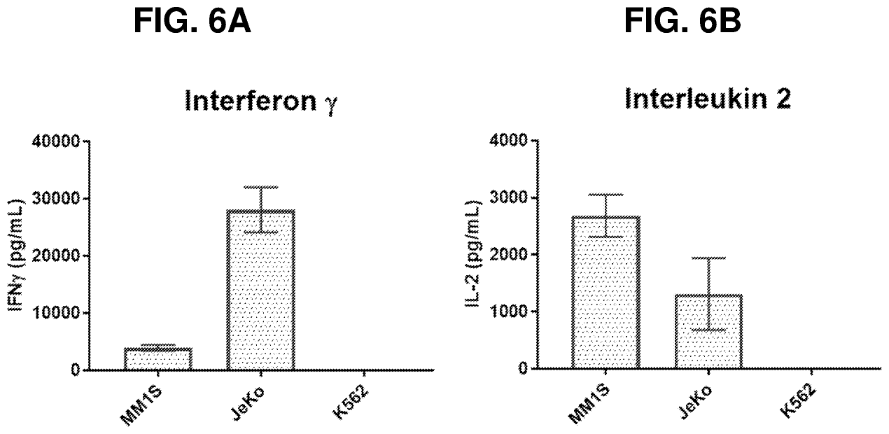

Tumor volume was measured twice weekly Animals treated with CTX120 cells demonstrated complete eradication of tumor burden, while tumors in untreated animals reached a tumor volume exceeding 1500 mm³ by the end of the study duration (FIG. 5).

### Example 4: Evaluation of Safety and Tolerability of the Anti-BCMA CAR T Cells

The selectivity of CTX120 cells for activation in response to BCMA-expressing cells and tissues was evaluated. To do so, the humanized mouse antibody, from which the scFv portion of the CTX120 CAR was derived, was evaluated for cross-reactivity to human tissues. Briefly, a standard panel of 32 human tissues (Adrenal, Bladder, Blood cells, Bone Marrow, Breast, Brain—cerebellum, Brain—cerebral cortext, Colon, Endothelium—blood vessels, eye, fallopian tube, GI: Tract: stomach, GI Tract small intestine, Heart, Kidney—glomerulus, Kidney—tubule, Liver, lung, lymph node, Nerve—peripheral, ovary, pancreas, parathyroid, parotid (salivary) gland, Pituitary, placenta, prostate, skin, spinal cord, spleen, striated muscle, testis, thymus, thyroid, tonsil, ureter, uterus—cervix, uterus—endometrium) was evaluated for binding of the antibody following exposure to two concentrations of antibody: an optimal concentration (5.0 μg/mL) and a high concentration (50.0 μg/mL). Binding was evaluated by an immunohistochemistry-based assay, wherein tissue staining was evaluated by a pathologist and positive staining was indicative of reactivity of the antibody to the tissue. As a positive control, staining was evaluated against purified BCMA protein absorbed to a tissue slide. For each tissue tested for antibody binding, tissue sections from three different human donors were evaluated. While robust staining was observed against the purified BCMA protein, no positive staining was observed in any of the human tissues. Thus, the antigen-binding scFv of the anti-BCMA CAR is highly-selective for tissues expressing BCMA.

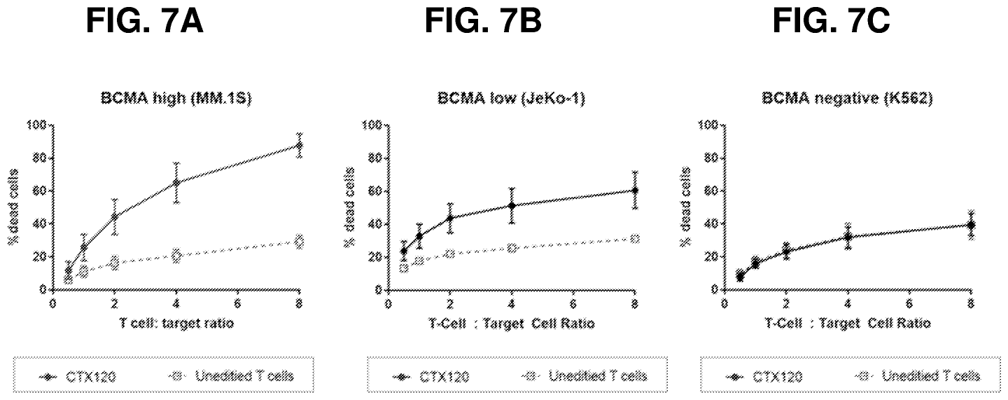

The selectivity of CTX120 cells for activation in response to BCMA-expressing cell lines was evaluated in vitro. To do so, CTX120 cells were co-cultured for 24 hours with 50,000 target cells with high BCMA expression (MM.1S cells), low BCMA expression (Jeko-1 cells), or no BCMA expression (K562 cells) at a ratio of 2:1 CART cells to target cells. Levels of IFNγ and IL-2 that were produced by activated anti-BCMA CAR T cells were measured in the co-culture supernatant using a Luminex-based assay (Milliplex, Millipore Sigma, MA, USA). Cytokine production in response to co-culture with target cells was evaluated for CTX120 cells derived from four individual donors, with the average±the standard error shown in FIGS. 6A-6B. As shown, no cytokine expression was measured when CTX120 cells were co-cultured with K562 cells that lack BCMA expression. In contrast, significant levels of both IFNγ and IL-2 were measured in co-cultures of CTX120 cells co-cultured with BCMA-expressing MM.1S or JeKo-1 cells (FIGS. 6A-6B).

Further, the selectivity of CTX120 cells for inducing target cell killing of BCMA-expressing cell lines was evaluated in vitro. To do so, CTX120 cells or unedited T cells were co-cultured for 24 hours with 50,000 target cells (e.g., MM.1S, JeKo-1 or K562 cells) at a ratio of 8:1, 4:1, 2:1, 1:1, or 0.5:1 T cells to target cells. Prior to co-culture, the target cells were labeled with 5 μM efluor670 (eBiosciences). Following co-culture, the cells were washed, suspended in 200 μL media containing a 1:500 dilution of 4′,6-diamidino-2-phenylindole (DAPI, Molecular Probes) for enumeration of dead/dying cells. 25 μL of CountBright beads (Life technologies) was added per sample. Cells were assessed for labeling by flow cytometry, and the percentage of target cells succumbing to cell lysis was determined using the following calculation:

Cells/μL=((number of live target cell events)/(number of bead events))×((Assigned bead count of lot(beads/50 μL))/(volume of sample))

Total target cells were calculated by multiplying cells/μL×the total volume of cells. The percent cell lysis was then calculated with the following equation:

% Cell lysis=(1−((Total Number of Target Cells in Test Sample)/(Total Number of Target Cells in Control Sample))×100.

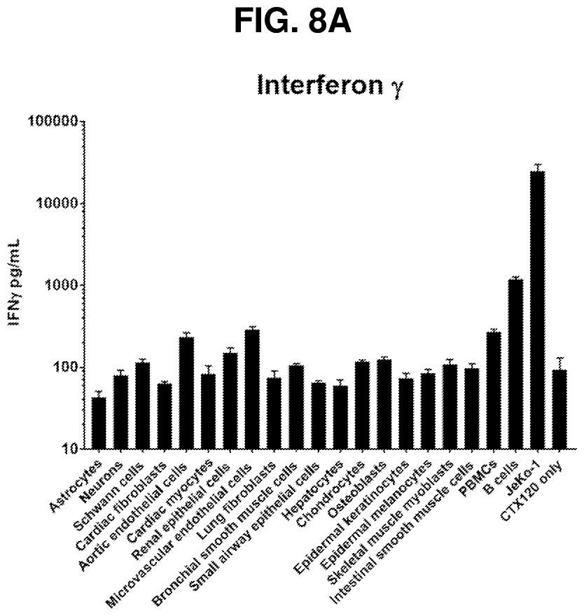

Cell killing was evaluated for unedited and edited T cells derived from four different donors, with the average % cell lysis±the standard deviation shown in FIGS. 7A-7C. For cells expressing BCMA (MM.1S in FIG. 7A and JeKo-1 in FIG. 7B), cell lysis induced by edited CTX120 cells was significantly higher than that induced by unedited T cells, even at low T cell to target cell ratios. In contrast, no difference in cell lysis was observed between unedited and edited T cells for K562 cells lacking BCMA expression (FIG. 7C). Thus, cytotoxicity induced by CTX120 cells is dependent upon expression of BCMA by the target cell.

The potential for primary non-tumor human cells to activate CTX120 cells was further evaluated. Of the primary human cells, only B cells are expected to comprise BCMA expressing cells. Activation of CTX120 cells was measured by quantifying levels of IFNγ and IL-2 following co-culture with primary human cells listed in Table 6 below.

TABLE 6
Primary Human Cells Evaluated for the Ability to Activate CTX120 Cells

| Organ | Cell Type |
|---|---|
| Central Nervous System | Astrocytes |
| | Neurons |
| | Schwann |
| Heart | Cardiac fibroblasts |
| | Aortic endothelial |
| | Cardiac myocytes |
| Kidney | Renal epithelial |
| Lung | Microvascular endothelial cells |
| | Lung fibroblasts |
| | Bronchial smooth muscle |
| | Small airway epithelial |
| Liver | Hepatocytes |
| Bone | Chondrocytes |
| | Osteoblasts |
| Skin | Epidermal keratinocytes |
| | Epidermal melanocytes |
| Skeletal muscle | Skeletal muscle myoblasts |
| Intestinal | Intestinal smooth muscle cells |
| Blood | PBMCs |
| | B cells |

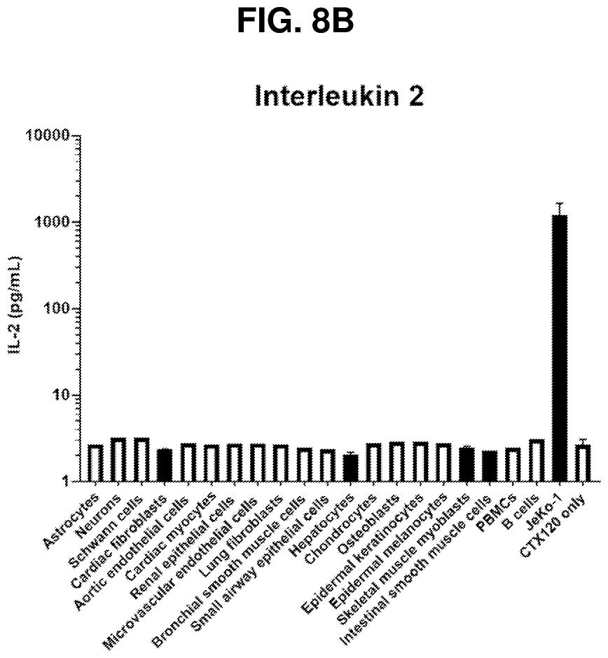

To do so, primary human cells were seeded at 25,000 cells per well in 96-well flat-bottom plates in preferred media and incubated overnight. After 24 hours, the primary cell media was removed, and 50,000 CTX120 cells were added in T cell growth media. Co-cultures were incubated for 24 hours, and assayed for production of IFNγ and IL-2 using a Luminex-based assay (Milliplex, Millipore Sigma, MA, USA). As a positive control, activation of CTX120 cells was evaluated in response to cells with low BCMA expression (e.g., Jeko-1 cells). The average±the standard deviation production of IFNγ and IL-2 is shown in FIG. 8A and FIG. 8B respectively. Open bars indicated that the values were below the limit of quantification. As shown in FIG. 8A, no co-culture between primary human cells and CTX120 cells resulted in significant secretion of IFNγ when compared to the Jeko-1 positive control cell line, except for co-culture with primary B cells that are known to comprise CD19+/BCMA⁺ cells. As shown in FIG. 8B, no-culture resulted in significant IL-2 production as compared to the Jeko-1 positive control. Based upon this outcome, CTX120 cells are not activated in the presence of normal, non-BCMA expressing human cells.

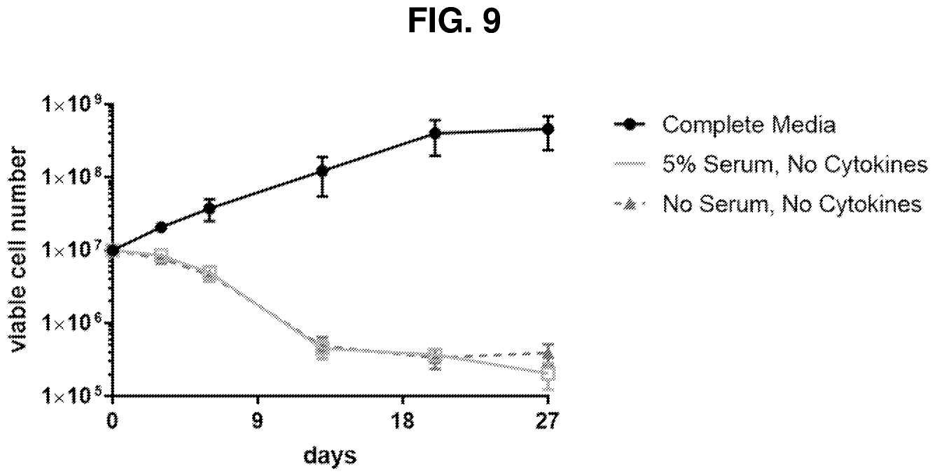

Transformed cells proliferate in a cytokine-independent manner Thus, to determine whether gene-editing results in oncogenic transformation, CTX120 cells were evaluated for the ability to grow in the absence of cytokines. To do so, the growth of CTX120 cells in ex vivo culture was evaluated over 27 days in complete media comprising serum and the cytokines IL-2 and IL-7, in media comprising serum but lacking cytokines (e.g., no IL-2 or IL-7), or in media lacking both serum and cytokines (e.g., no serum, IL-2, or IL-7). 5×10⁶ CTX120 cells were plated at approximately 2 weeks following gene-editing (day 0). At various time points, the number of viable CTX120 cells was enumerated using flow cytometry. While T cell growth plateaued when cultured in complete media, the number of viable T cells decreased over time when grown in media lacking cytokines (either with or without serum) as shown in FIG. 9. Shown is the average number of viable cells±the standard error for CTX120 cells derived from four different donors. Thus, the gene-editing approach used to generate CTX120 cells does not result in undesirable oncogenic transformation.

### Example 5: Analysis of Immune Reactivity with Administration of Anti-BCMA CAR T Cells

The potential for unedited T cells and edited CTX120 cells to cause GvHD following a single dosage was evaluated in mice. Edited CTX120 cells were prepared as described in Example 1. The CTX120 anti-BCMA CAR does not recognize mouse BCMA. However, evaluation for GvHD symptoms in mice (e.g., weight loss, decreased survival, and/or increased morbidity) in response to treatment with unedited or edited T cells is indicative of a GvHD toxicity induced by off-target reactivity of the T cells (e.g., due to TCR reactivity towards alloantigens). As a positive control, mice were treated with unedited allogeneic T cells that cause GvHD toxicity due to reactivity of the TCR with mouse tissue antigens. Treatment with allogeneic CTX120 cells that have very low expression of TCR was evaluated for inducing GvHD toxicity.

To evaluate a GvHD response, NSG mice (NOD.Cg-Prkdc^{scid}I12rg^{tm1Wjl}/SzJ) were first exposed to total body irradiation (total irradiation dosage of 200 cGy), then treated with vehicle only (e.g., no T cells), unedited T cells, or edited CTX120 cells (e.g., TCR⁻ β2M⁻ CAR⁺ T cells) as shown in Table 7. T cells were administered approximately 6 hours post radiation on day 1 in a 250 μL volume of phosphate-buffered saline (PBS) via an intravenous slow bolus injection. Radiation was delivered at a rate of 160 cGy/min.

TABLE 7
Design of an In Vivo Study to Evaluate GvHD Response to CTX120

| | | | | Number |
|---|---|---|---|---|
| | | | | of |
| | | Tcell | Total | animals |
| | T cell dosage | concentration | irradiation | (Male/ |
| Group | (cells/mouse) | (cells/mL) | dosage | Female) |
| Vehicle - no radiation | 0 | 0 | 0 cGy | 5/5 |
| Vehicle - radiation | 0 | 0 | 200 cGy | 15/15 |
| Unedited T cells | 1 × 10⁷ | 4 × 10⁷ | | 15/15 |
| CTX120 cells | 4 × 10⁷ | 8 × 10⁷ | | 15/15 |

Following treatment, the animals were evaluated for up to 84 days after radiation for survival, appearance of GvHD symptoms, and body weight. GvHD symptoms were defined as changes to the skin (e.g., pallor and/or redness), decreased activity, hunched back posture, slight to moderate thinness, and increased respiratory rate.

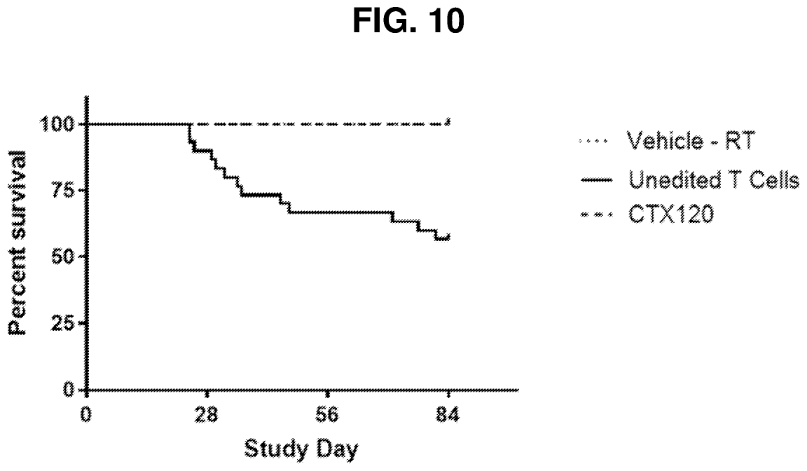

No mortality was observed in untreated animals or animals exposed to radiation alone or radiation combined with a dosage of CTX120 cells. However, significant mortality was observed for animals receiving radiation in combination with a dosage of unedited T cells as shown in FIG. 10. Additionally, weight loss was observed in several animals treated with unedited T cells, but not in animals treated with vehicle or CTX120 cells. Additionally, no GvHD symptoms were observed in animals treated with CTX120 cells. Thus, these results confirm that CTX120 cells edited to eliminate TCR-expressing cells do not induce off-target reactivity that results in a GvHD response.

Alloreactivity towards human cells was compared for unedited T cells and T cells edited to be TCR and β2M negative according to the gene-editing methods described in Example 1. Specifically, primary human T cells were electroporated with Cas9-sgRNA RNP complexes targeting the TRAC and β2M gene loci. However, the cells were not treated with rAAV encoding an anti-BCMA CAR, thus providing a population of cells comprising T cells with a disrupted TRAC and β2M gene (TRAC⁻/β2M⁻ T cells) for use in evaluating the effect of a TCR knockout on alloreactivity.

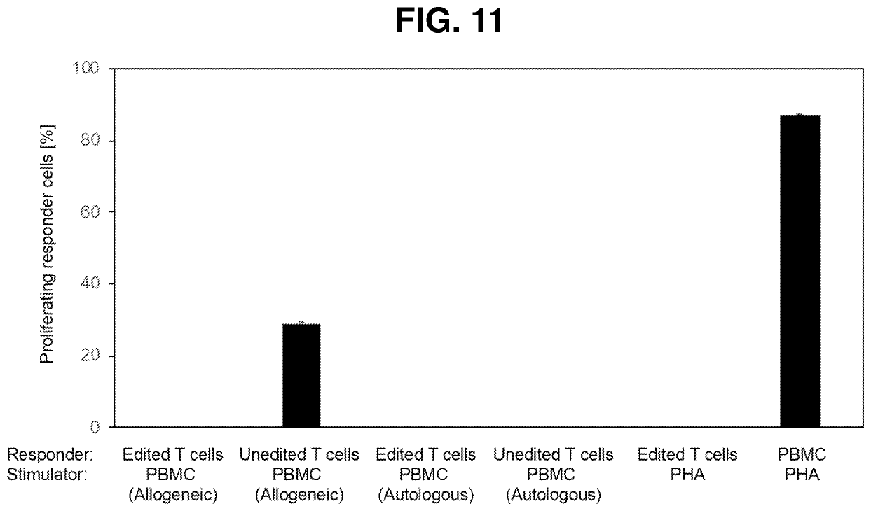

To evaluate alloreactivity, unedited T cells or edited T cells were incubated with PBMCs that were derived from the same donor (e.g., autologous or matched PBMCs) or a different donor (e.g., allogeneic or unmatched PBMCs) and activation was evaluated by measuring T cell proliferation using a flow cytometry-based assay measuring incorporation of 5-ethynyl-2′-deoxyuridine (EdU: Invitrogen) according to the manufacturer's protocol. As a positive control, T cells were treated with phytohaemagglutinin-L (PHA) that functions to cross-link the TCR and induce T cell activation. Treatment with PHA resulted in robust proliferation in unedited T cells, but as expected, not in edited T cells that lack TCR expression (FIG. 11). Also, as expected, neither edited nor unedited T cells proliferated in the presence of autologous PBMCs. However, unedited T cells proliferate in the presence of allogenic PBMCs, indicating alloreactivity to unmatched human cells. In contrast, edited T cells demonstrated no proliferation in response to allogeneic PBMCs (FIG. 11). Thus, loss of TCR expression in edited T cells corresponds to lack of activation in response to unmatched human cells.

### Example 6. A Phase I Dose Escalation and Cohort Expansion Study of Safety and Efficacy of Anti-BCMA Allogenic CRISPR-Cas9-Engineered T Cells (CTX120) in Subjects with Relapsed or Refractory Multiple Myeloma

This study evaluates the safety, efficacy, pharmacokinetics, and pharmacodynamic effects of CTX120, an allogeneic chimeric antigen receptor (CAR) T cell therapy directed towards B cell maturation antigen (BCMA) in subjects with relapsed or refractory multiple myeloma (MM).

MM is a malignancy of terminally differentiated plasma cells in the bone marrow that represents about 10% of all hematologic malignancies, and is the second most common hematologic malignancy after non-Hodgkin lymphoma (Kumar et al., 2017, Leukemia 31, 2443-2448; Rajkumar and Kumar, 2016, Mayo Clin Proc 91, 101-119).

CTX120 is a BCMA-directed T cell immunotherapy comprised of allogeneic T cells that are genetically modified ex vivo using CRISPR-Cas9 gene editing components (sgRNA and Cas9 nuclease). The modifications include disruption of the T cell receptor alpha constant (TRAC) and beta-2 microglobulin (B2M) loci, and the simultaneous insertion of an anti-BCMA CAR transgene into the TRAC locus. The CAR is comprised of a humanized scFv specific for BCMA, followed by a CD8 hinge and transmembrane region that is fused to the intracellular signaling domains for CD137 (4-1BB) and CD3. The gene knockouts are intended to reduce the probability of GvHD, redirect the modified T cells towards BCMA-expressing tumor cells, and increase the persistence of the allogeneic cells.

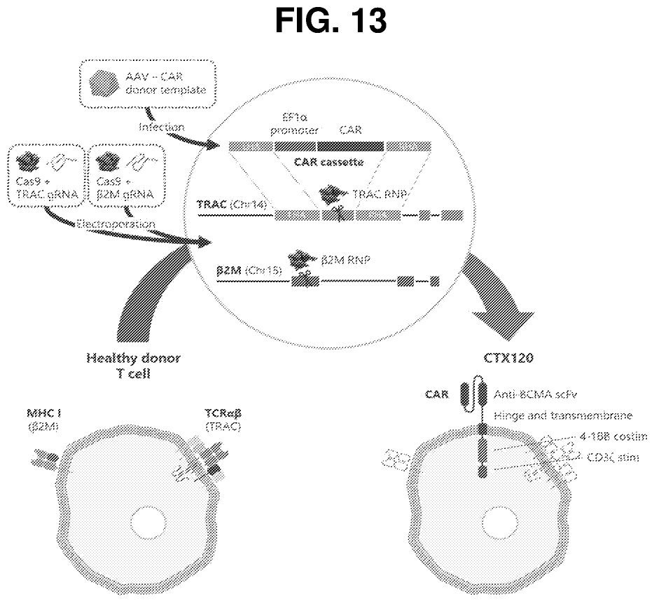

CTX120 is prepared from healthy donor peripheral blood mononuclear cells obtained via a standard leukapheresis procedure. The mononuclear cells are enriched for T cells and activated with anti-CD3/CD28 antibody—coated beads, then electroporated with CRISPR-Cas9 ribonucleoprotein complexes, and transduced with a CAR gene—containing recombinant adeno-associated virus (AAV) vector. The modified T cells are expanded in cell culture, purified, formulated into a suspension, and cryopreserved. The product is stored onsite and thawed immediately prior to administration. CTX120 production is summarized in FIG. 13.

The specificity and antitumor cytotoxicity of CTX120 was assessed using in vitro and in vivo pharmacology studies. CTX120 cells released effector cytokines when cocultured with BCMA⁺ tumor cells in vitro resulted in tumor cell death. CTX120 inhibited tumor growth in vivo in human tumor xenograft mouse models. In vitro and in vivo safety assessments were performed to assess the risk of immune reactivity and oncogenesis. No off target edits were identified. Safety studies demonstrated that CTX120 did not cause any clinical or histopathological GvHD in mice and confirmed that CTX120 cells did not grow in the absence of cytokines after gene editing.

This first-in-human trial in subjects with relapsed or refractory multiple myeloma evaluates the safety and efficacy of this CRISPR-Cas9-modified allogeneic CAR T cell approach.

### 1. Study Objectives

Primary objective, Part A (dose escalation): assesses the safety of escalating doses of CTX120 in combination with various lymphodepleting and immunomodulatory agents in subjects with relapsed or refractory multiple myeloma to determine the maximum tolerated dose (MTD) and/or recommended dose for cohort expansion.

Primary objective, Part B (cohort expansion): assesses the efficacy of CTX120 in subjects with relapsed or refractory multiple myeloma, as measured by ORR according to International Myeloma Working Group (IMWG) response criteria (Kumar et al., 2016). Secondary objectives further characterize the efficacy, safety, and pharmacokinetics of CTX120. Exploratory objectives identify genomic, metabolic, and/or proteomic biomarkers associated with CTX120 that may indicate or predict clinical response, resistance, safety, disease, or pharmacodynamic activity.

Secondary objectives (Parts A and B): To further characterize the efficacy, safety, and pharmacokinetics of CTX120.

Exploratory objectives (Parts A and B): To identify genomic, metabolic, and/or proteomic biomarkers associated with CTX120 that may indicate or predict clinical response, resistance, safety, disease, or pharmacodynamic activity.

### 2. Subject Eligibility

2.1 Inclusion Criteria

To be considered eligible to participate in this study, a subject must meet all the inclusion criteria listed below:

1. Age≥18 years

2. Able to understand and comply with protocol-required study procedures and voluntarily sign a written informed consent document

3. Diagnosis of multiple myeloma with relapsed or refractory disease, as defined by IMWG response criteria (Table 22 below), and at least 1 of the following:
a) Have had at least 2 prior lines of therapy, including an IMiD (e.g., lenalidomide, pomalidomide), PI (e.g., bortezomib, carfilzomib), and a CD38-directed monoclonal antibody (e.g., daratumumab; if approved and available in country/region)
b) Multiple myeloma that is triple-refractory, defined as progression on or within 60 days of treatment with PI, IMiD, and anti-CD38 antibody, as part of the same or different regimens; or multiple myeloma that is double-refractory to PI and IMiD, as part of the same or different regimens.
c) Multiple myeloma relapsed within 12 months after autologous SCT
d) At least 1 of the above criteria (3a, b, or c) AND previously received a CD38-directed monoclonal antibody

4. Measurable disease, including at least 1 of the following criteria:
Serum M-protein≥0.5 g/dL
Urine M-protein≥200 mg/24 hours
Serum free light chain (FLC) assay: Involved FLC level≥10 mg/dL (100 mg/L) provided serum FLC ratio is abnormal

5. Eastern Cooperative Oncology Group (ECOG) performance status 0 or 1 (Appendix B)

6. Meets criteria to undergo LD chemotherapy and CAR T cell infusion.

7. Adequate organ function:
Renal: Estimated glomerular filtration rate>50 mL/min/1.73 m2
Liver: Aspartate transaminase or alanine transaminase<3×upper limit of normal (ULN); total bilirubin<2×ULN
Cardiac: Hemodynamically stable and left ventricular ejection fraction≥45% by echocardiogram
Pulmonary: Oxygen saturation level on room air >91% per pulse oximetry

8. Female subjects of childbearing potential (postmenarcheal with an intact uterus and at least 1 ovary, who are less than 1 year postmenopausal) must agree to use acceptable method(s) of contraception from enrollment through at least 12 months after CTX120 infusion.

9. Male subjects must agree to use effective contraception from enrollment through at least 12 months after CTX120 infusion.

2.2 Exclusion Criteria

To be eligible for entry into the study, the subject must not meet any of the exclusion criteria listed below:

1. Prior allogeneic SCT

2. Less than 60 days from autologous SCT at time of screening and with unresolved serious complications

3. Plasma cell leukemia (>2.0×109/L circulating plasma cells by standard differential), or nonsecretory MM, or Waldenström's macroglobulinemia or POEMS (polyneuropathy, organomegaly, endocrinopathy, monoclonal protein, and skin changes) syndrome, or amyloidosis with end organ involvement and damage

4. Prior treatment with any of the following therapies:
Any gene therapy or genetically modified cell therapy, including CAR T cells or natural killer cells
Prior treatment with BCMA-directed therapy, including BCMA-directed antibody, bispecific T cell engager, or antibody-drug conjugate
Radiation therapy within 14 days of enrollment. Palliative radiation therapy for symptom management is permitted.

5. Known contraindication to cyclophosphamide, fludarabine, or any of the excipients of CTX120 product

6. Evidence of direct central nervous system (CNS) involvement by multiple myeloma

7. History or presence of clinically relevant CNS pathology such as a seizure disorder, cerebrovascular ischemia/hemorrhage, dementia, cerebellar disease, any autoimmune disease with CNS involvement, or another condition that may increase CAR T cell-related toxicities

8. Unstable angina, clinically significant arrhythmia, or myocardial infarction within 6 months of enrollment

9. Presence of bacterial, viral, or fungal infection that is uncontrolled or requires IV anti-infectives

10. Positive for presence of human immunodeficiency virus (HIV) type 1 or 2, or active hepatitis B virus (HBV) or hepatitis C virus (HCV) infection. Subjects with prior history of HBV or HBC infection who have documented undetectable viral load (by quantitative polymerase chain reaction [PCR] or nucleic acid testing) are permitted. Infectious disease testing (HIV-1, HIV-2, HCV antibody and PCR, HBV surface antigen, HBV surface antibody, HBV core antibody) performed within 30 days of signing the informed consent form (ICF) may be considered for subject eligibility.

11. Previous or concurrent malignancy, except basal cell or squamous cell skin carcinoma, adequately resected and in situ carcinoma of cervix, or a previous malignancy that was completely resected and has been in remission for ≥5 years

12. Received live vaccine within 28 days of enrollment

13. Use of systemic antitumor therapy or investigational agent within 14 days prior to enrollment. Use of physiological doses of steroids (e.g., ≤10 mg/day prednisone or equivalent) is permitted for subjects previously on steroids if clinically indicated.

14. Primary immunodeficiency disorder or active autoimmune disease requiring steroids and/or other immunosuppressive therapy

15. Diagnosis of significant psychiatric disorder or other medical condition that could impede the subject's ability to participate in the study

16. Women who are pregnant or breastfeeding

### 3. Study Design

3.1 Investigational Plan

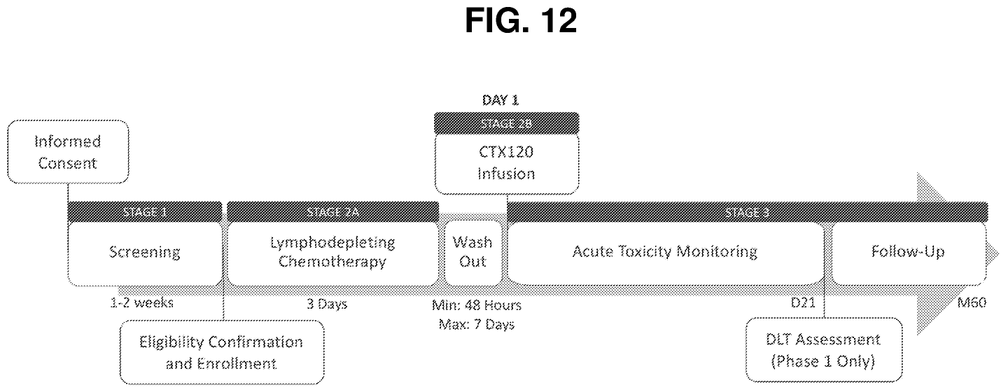

This is an open-label, multicenter, Phase 1 study evaluating the safety and efficacy of escalating doses of CTX120 in combination with various LD and immunomodulatory agents in subjects with relapsed or refractory multiple myeloma (Table 8 below). The study is divided into 2 parts: dose escalation (Part A), followed by cohort expansion (Part B). A schematic illustration of the treatment schedule is provided in FIG. 12.

In Part A, dose escalation begins in adult subjects with 1 of the following: relapsed or refractory MM after at least 2 prior lines of therapy, including an IMiD, PI, and CD38-directed monoclonal antibody; progressive MM that is triple-refractory to PI, IMiD, and anti-CD38 antibody; or MM relapsed within 12 months after autologous SCT. Dose escalation is performed according to the criteria outlined below.

In Part B, an expansion cohort is to be initiated to further assess the safety and efficacy of CTX120 using an optimal Simon 2-stage design. In the first stage, up to 27 subjects are to be enrolled and treated with the recommended dose of CTX120 for Part B cohort expansion (at or below the MTD determined in Part A).

3.1.1 Study Design

During both dose escalation (Part A) followed by cohort expansion (Part B), the study consists of 3 main stages as follows:
Stage 1: Screening to determine eligibility for treatment (1-2 weeks).
Stage 2: Treatment (Stage 2A and Stage 2B); see Table 2 for treatment by cohort (1-2 weeks)
Stage 3: Follow-up for all cohorts (5 years)

Part A investigates escalating doses of CTX120 in multiple independent cohorts. These cohorts allow preliminary evaluation of the safety and pharmacokinetics of CTX120 when used with different LD and immunomodulatory agents, as summarized in the following Table 8.

TABLE 8
Part A Dose Cohort

| Cohort | Treatment (Stage 2A + Stage 2B) |
|---|---|
| A | Stage 2A |
| | LD chemotherapy: Co-administration of fludarabine 30 mg/m² + cyclophosphamid |
| | 300 mg/m² IV daily for 3 days. Both agents should be started on the same day and |
| | administered for 3 consecutive days. LD chemotherapy must be completed at least 4 |
| | hours (but no more than 7 days) prior to CTX120 infusion. |
| | Stage 2B |
| | CTX120 starting at DL1. Administered at least 48 hours (but no more than 7 days) |
| | after completion of the 3-day course of LD chemotherapy. |
| B | Stage 2A |
| | LD chemotherapy: Co-administration of fludarabine 30 mg/m² + cyclophosphamid |
| | 500 mg/m² IV daily for 3 days. Both agents should be started on the same day and |
| | administered for 3 consecutive days. LD chemotherapy must be completed at least 4 |
| | hours (but no more than 7 days) prior to CTX120 infusion. |
| | Stage 2B |
| | CTX120 starting at DL2 or DL3. Administered at least 48 hours (but no more than 7 |
| | days) after completion of the 3-day course of LD chemotherapy. |

DL1/2/3 : Dose Level 1, 2, or 3;
IV: intravenous(ly);
LD: lymphodepleting.
Notes:
Subjects should meet the criteria specified in herein prior to both the initiation of LD chemotherapy and infusion of CTX120.
CTX120 infusion may begin at DL1 for Cohort A and may begin at DL2 or DL3 for Cohort B after assessment.

During the post-CTX120 infusion period, subjects are monitored for acute toxicities, including CRS, neurotoxicity, GvHD, and other adverse events (AEs). Toxicity management guidelines are provided below. During Part A (dose escalation), all subjects are hospitalized for observation for the first 7 days following CTX120 infusion. In Parts A and B, the length of hospitalization for observation may be extended where required by local regulation or site practice. In both Parts A and B, subjects must remain within proximity of the investigative site (i.e., 1-hour transit time) for 28 days after CTX120 infusion.

3.1.2. Study Subjects

Approximately 6 to 60 subjects are treated in Part A (dose escalation). Approximately 70 subjects are treated in Part B (cohort expansion).

3.1.3. Study Duration

Subjects participate in this study for 5 years. After completion of this study, all subjects are required to participate in a separate long-term follow-up study for an additional 10 years to assess long-term safety and survival.

3.2 CTX120 Dose Escalation

Dose escalation is performed using a standard 3+3 design, in which 3 to 6 subjects are to be enrolled at each dose level depending on the occurrence of DLT, as defined herein. The DLT evaluation period begins with CTX120 infusion and last for 28 days.

Table 9 lists the CAR+ T cell doses of CTX120, based on the total number of CAR+ T cells that may be evaluated in this study, beginning with DL1 for Cohort A. Dose levels for Cohort B may be initiated after the corresponding dose level (e.g., DL2 or DL3) in Cohort A has completed the DLT evaluation period for all subjects. If 1 of 3 subjects in DL4 experiences a DLT, the treatment may expand to treat 3 more subjects at DL4 or de-escalate to a lower dose level consisting of 6×10⁸ CAR+ T cells. In addition, the dose of CTX120 may escalate to the dose level with 6×10⁸ CAR+ T cells or DL4 after evaluating the data from DL3. Dosing can be staggered between the 1^{st} and the 2^{nd} subject within each cohort at the starting dose level and/or at subsequent dose levels, such that the 2^{nd} subject in each dose level only receives CTX120 when the 1^{st} subject has completed the DLT evaluation period.

TABLE 9
Dose Escalation of CTX120

| Dose Level | Total CAR+ T Cell Dose |
|---|---|
| −1 (de-escalation) | 2.5 × 10⁷ |
| 1 | 5 × 10⁷ |
| 2 | 1.5 × 10⁸ |
| 3 | 4.5 × 10⁸ |
| 4 | 7.5 × 10⁸* |

CAR: chimeric antigen receptor.
*A lower dose level consisting of 6 × 10⁸ CAR+ T cells may be used for de-escalation from Dose Level 4.

In DL1 (and DL−1, if required), subjects are treated in a staggered manner, such that the 2nd and 3rd subjects only receive CTX120 once the previous subject has completed the DLT evaluation period. If DL1 or −1 is expanded after 3 subjects, the additional 3 subjects in the cohort may be enrolled and dosed concurrently. If no DLT occurs at DL1, dose escalation progresses to the subsequent level. For subsequent Dose Levels 2, 3, and 4, dosing between the 1^{st} and 2^{nd} subject are staggered by 28 days for each dose level.

Dose escalation is performed according to the following rules:
If 0 of 3 subjects experience a DLT, escalate to the next dose level.
If 1 of 3 subjects experiences a DLT, expand the current dose level to 6 subjects.
   If 1 of 6 subjects experiences a DLT, escalate to the next dose level.
   If ≥2 of 6 subjects experience a DLT:
      If in DL−1, evaluate alternative dosing schema or declare inability to determine recommended dose for Part B cohort expansion.
      If in DL1, de-escalate to DL−1.
      If in DL2, DL3, or DL4 declare previous dose level as the MTD.
If ≥2 of 3 subjects experience a DLT:
   If in DL−1, evaluate alternative dosing schema or declare inability to determine the recommended dose for Part B cohort expansion.
   If in DL1, decrease to DL−1.
   If in DL2, DL3, or DL4, declare previous dose level the MTD.
No dose escalation beyond highest dose listed in Table 9 above.

At least 6 subjects are administered CTX120 before a recommended dose for Part B cohort expansion is declared.

3.2.1 Maximum Tolerated Dose Definition

The MTD is the highest dose for which DLTs are observed in less than 33% of subjects. An MTD may not be determined in this study. A decision to move to the Part B expansion cohort may be made in the absence of an MTD provided the dose is at or below the maximum dose studied in Part A of the study.

3.2.2 DLT Definitions

Toxicities are graded and documented according to National Cancer Institute Common Terminology Criteria for Adverse Events (CTCAE) version 5, with the following exceptions:
CRS:
   Part A: Lee criteria (Lee et al., 2014, Blood 124, 188-195)
   Part B: American Society for Transplantation and Cellular Therapy (ASTCT) criteria (Lee et al., 2019, Biol Blood Marrow Transplant 25, 625-638)
Neurotoxicity, Parts A and B:
   CTCAE v5.0
   Immune effector cell-associated neurotoxicity syndrome (ICANS) criteria (Lee et al., 2019)
GvHD, Parts A and B:
   Mount Sinai Acute GVHD International Consortium (MAGIC) criteria (Harris et al., 2016, Biol Blood Marrow Transplant 22, 4-10)

AEs that have no plausible causal relationship with CTX120 are be considered DLTs.

A DLT is defined as any of the following CTX120-related events occurring during the DLT evaluation period that persists beyond the specified duration (relative to the time of onset):
A. Grade 4 CRS
B. Grade 3 or 4 neurotoxicity (based on ICANS criteria)
C. Grade≥2 GvHD that is steroid-refractory (e.g., progressive disease after 3 days of steroid treatment [e.g., 1 mg/kg/day], or having no response after 7 days of treatment)
D. Death during the DLT period (except due to disease progression)
E. Any CTX120-related grade≥3 vital organ toxicity (e.g., pulmonary, cardiac) of any duration, except as listed below.

The following are NOT be considered as DLTs:
1. Grade 3 CRS that improves to grade≤2 within 72 hours
2. Grade≤3 tumor lysis syndrome lasting <7 days
3. Grade 3 or 4 fever
4. Grade≥3 allergic reaction improving to grade≤2 within 48 hours of instituting supportive care
5. Grade 3 fatigue lasting <7 days
6. Bleeding in the setting of thrombocytopenia (platelet count<50×109/L); documented bacterial infections or fever in the setting of neutropenia (absolute neutrophil count [ANC]<1000/mm3)
7. Grade 3 or 4 hypogammaglobulinemia
8. Grade 3 or 4 liver function studies that improve to grade≤2 within 7 days.
9. Grade 3 or 4 renal insufficiency that improves to grade≤2 within 7 days.
10. Grade 3 or 4 cardiac arrythmia that improves to grade≤2 within 48 hours.
11. Grade 3 or 4 pulmonary toxicity that resolves to grade≤2 within 72 hours. Grade 3 or 4 events that are isolated, CTX120-related, and not secondary to supportive treatment as part of CRS are considered DLTs.
12. Grade 3 or 4 thrombocytopenia or neutropenia is assessed retrospectively. After at least 6 subjects are infused, if ≥50% of subjects have prolonged cytopenias (i.e., lasting more than 28 days postinfusion), dose escalation is to be suspended. Grade≥3 cytopenias that were present at the start of LD chemotherapy may not be considered a DLT. Another etiology may be identified.

### 4. Study Treatment

4.1 Lymphodepleting (LD) Chemotherapy

All subjects receive LD chemotherapy prior to the infusion of CTX120. LD chemotherapy consists of: (1) Fludarabine 30 mg/m² IV daily for 3 doses, and (2) Cyclophosphamide 300 mg/m² IV daily for 3 doses.

Both agents are started on the same day and administered for 3 consecutive days for all cohorts. Subjects should start LD chemotherapy within 7 days of study enrollment. Adult subjects with moderate impairment of renal function (creatinine clearance [CrCl] 30-70 mL/min/1.73 m2) should have a 20% dose reduction of fludarabine and be monitored closely per the applicable prescribing information.

Both LD chemotherapy agents are started on the same day and administered for 3 consecutive days. Subjects should start LD chemotherapy within 7 days of study enrollment.

Reference the local prescribing information for fludarabine and cyclophosphamide for guidance regarding the storage, preparation, administration, supportive care instructions, and toxicity management associated with LD chemotherapy.

LD chemotherapy may be delayed if any of the following signs or symptoms are present:
Significant worsening of clinical status that increases the potential risk of AEs associated with LD chemotherapy
Requirement for supplemental oxygen to maintain a saturation level>91%
New uncontrolled cardiac arrhythmia
Hypotension requiring vasopressor support
Active infection: Positive blood cultures for bacteria, fungus, or virus not responding to treatment
Neurotoxicity known to increase risk of ICANS (e.g., seizures, stroke, change in mental status). Neurotoxicity of benign origin (e.g., headache), lasting less than 48 hours and considered reversible may be allowed.

4.2. Administration of CTX120

CTX120 consists of allogeneic T cells modified with CRISPR-Cas9, resuspended in cryopreservative solution (CryoStor CS-5), and supplied in a 6-mL infusion vial. A flat dose of CTX120 (based on number of CAR⁺ T cells) is administered as a single IV infusion. The total dose may be contained in multiple vials. Infusion should preferably occur through a central venous catheter. A leukocyte filter must not be used.

Prior to the start of CTX120 infusion, the site pharmacy ensures that 2 doses of tocilizumab and emergency equipment are available for each specific subject treated. Subjects are premedicated per the site standard of practice with acetaminophen PO (i.e., paracetamol or its equivalent per site formulary) and diphenhydramine hydrochloride IV or PO (or another H1-antihistamine per site formulary) approximately 30-60 minutes prior to CTX120 infusion. Prophylactic systemic corticosteroids are not administered, as they may interfere with the activity of CTX120.

There is a dose limit of 7×10⁴ TCR⁺ cells/kg imposed for all dose levels. Based on the percentage of CAR⁺ cells in the CTX120 lot to be administered, enrollment at higher dose levels (e.g., DL4) can be restricted to subjects with a minimum weight to ensure the TCR⁺ cell limit is not exceeded. See below for medications that must be discontinued prior to CTX120 infusion.

CTX120 infusion may be delayed if any of the following signs or symptoms are present:
New active uncontrolled infection
Worsening of clinical status compared to prior to start of LD chemotherapy that places the subject at increased risk of toxicity
Neurotoxicity known to increase risk of ICANS (e.g., seizures, stroke, change in mental status). Neurotoxicity of benign origin (e.g., headache) lasting less than 48 hours and considered reversible is to be allowed.

CTX120 cells are administered at least 48 hours (but no more than 7 days) after the completion of LD chemotherapy. If CTX120 infusion is delayed by more than 10 days, LD chemotherapy must be repeated.

Refer to the Infusion Manual for detailed instructions on preparation, storage, handling, and administration of CTX120.

4.2.1 CTX120 Postinfusion Monitoring

Following CTX120 infusion, subjects' vitals should be monitored every 30 minutes for 2 hours after infusion or until resolution of any potential clinical symptoms.

Subjects in Part A are hospitalized for observation for a minimum of 7 days after CTX120 infusion. Postinfusion hospitalization in Part B are considered based on the safety information obtained during dose escalation and may be performed. In Part B, hospitalization for observation can be considered. In Parts A and B, the length of hospitalization for observation may be extended where required by local regulation or site practice. In both Parts A and B, subjects must remain in proximity of the investigative site (i.e., 1-hour transit time) for at least 28 days after CTX120 infusion. Management of acute CTX120-related toxicities should occur ONLY at the study site.

Subjects are monitored for signs of CRS, tumor lysis syndrome (TLS), neurotoxicity, GvHD, and other AEs according to the schedule of assessments (Table 18 and Table 19 below). Guidelines for the management of CAR T cell-related toxicities are described herein. Subjects should remain hospitalized until CTX120-related nonhematologic toxicities (e.g., fever, hypotension, hypoxia, ongoing neurological toxicity) return to grade 1. Subjects may remain hospitalized for longer periods.

4.3. Prior and Concomitant Medications

4.3.1 Allowed Medications

Necessary supportive measures for optimal medical care may be given throughout the study, including IV antibiotics to treat infections, growth factors, blood components, and bone-directed therapies (including zoledronic acid or denosumab), except for prohibited medications listed below.

All concurrent therapies, including prescription and nonprescription medication, and medical procedures must be recorded from the date of signed informed consent through 3 months after CTX120 infusion. Beginning 3 months post-CTX120 infusion, only the following selected concomitant medications may be collected: IV immunoglobulins, vaccinations, anticancer treatments (e.g., chemotherapy, radiation, immunotherapy), immunosuppressants (including steroids), bone-directed therapies, and any investigational agents.

4.3.2 Prohibited Medications

The following medications are prohibited during certain periods of the study as specified below:
Corticosteroid therapy at a pharmacologic dose (>10 mg/day of prednisone or equivalent doses of other corticosteroids) and other immunosuppressive drugs should be avoided after CTX120 administration unless medically indicated to treat new toxicity or as part of management of CRS or neurotoxicity associated with CTX120, as described herein.
Granulocyte-macrophage colony-stimulating factor (GM-CSF) following CTX120 infusion due to the potential to worsen symptoms of CRS
Care should be taken with administration of G-CSF following CTX120
Live vaccine within 28 days of enrollment to 3 months following CTX120 infusion
Any anticancer therapy (e.g., chemotherapy, immunotherapy, targeted therapy, radiation, or other investigational agents), or LD chemotherapy prior to disease progression. Palliative radiation therapy for symptom management is permitted depending on extent, dose, and site(s). Site(s), dose, and extent should be defined and reported to the medical monitor for determination.

### 5. Toxicity Management

5.1 General Guidance

Prior to LD chemotherapy, infection prophylaxis (e.g., antiviral, antibacterial, antifungal agents) should be initiated according to institutional standard of care for MM patients in an immunocompromised setting.

Subjects must be closely monitored for at least 28 days after CTX120 infusion. Significant toxicities have been reported with autologous CAR T cell therapies. Although this is a first-in-human study and the clinical safety profile of CTX120 has not been described, the following general recommendations are provided:
Fever is the most common early manifestation of CRS; however, subjects may also experience weakness, hypotension, or confusion as first presentation.
Diagnosis of CRS should be based on clinical symptoms and NOT laboratory values.
In subjects who do not respond to CRS-specific management, always consider sepsis and resistant infections. Subjects should be continually evaluated for resistant or emergent bacterial infections, as well as fungal or viral infections.
CRS, HLH, and TLS may occur at the same time following CAR T cell infusion. Subjects should be consistently monitored for signs and symptoms of all the conditions and managed appropriately.
Neurotoxicity may occur at the time of CRS, during CRS resolution, or following resolution of CRS. Grading and management of neurotoxicity may be performed separately from CRS.
Tocilizumab must be administered within 2 hours from the time of order.

In addition, signs of GvHD are to be monitored closely due to the allogeneic nature of CTX120 (see descriptions below).

5.2 Toxicity-Specific Guidance

5.2.1 Infusion Reactions

If an infusion reaction occurs, acetaminophen (paracetamol) and diphenhydramine hydrochloride (or another H1-antihistamine) may be repeated every 6 hours after CTX120 infusion.

Nonsteroidal anti-inflammatory medications may be prescribed as needed if the subject continues to have fever not relieved by acetaminophen. Systemic steroids should NOT be administered except in cases of life-threatening emergency, as this intervention may have a deleterious effect on CAR T cells.

5.2.2 Febrile Reaction and Infection Prophylaxis

Infection prophylaxis should occur according to the institutional standard of care for MM patients in an immunocompromised setting.

In the event of febrile reaction, an evaluation for infection should be initiated and the subject managed appropriately with antibiotics, fluids, and other supportive care as medically indicated and determined by the treating physician. Viral and fungal infections should be considered throughout a subject's medical management if fever persists. If a subject develops sepsis or systemic bacteremia following CTX120 infusion, appropriate cultures and medical management should be initiated. Additionally, consideration of CRS should be given in any instances of fever following CTX120 infusion within 30 days postinfusion.

Viral encephalitis (e.g., human herpes virus [HHV]-6 encephalitis) must be considered in the differential diagnosis for subjects who experience neurocognitive symptoms after receiving CTX120. A lumbar puncture (LP) is required for any Grade 3 or higher neurocognitive toxicity and is strongly recommended for Grade 1 and Grade 2 events. Whenever a lumbar puncture is performed, an infectious disease panel will review data from the following assessments (at a minimum): quantitative testing for HSV 1&2, Enterovirus, Human Parechovirus, VZV, CMV, and HHV-6. Lumbar puncture must be performed within 48 hours of symptom onset and results from the infectious disease panel must be available within 4 days of the LP in order to appropriately manage the subject.

5.2.3 Tumor Lysis Syndrome

Subjects receiving CAR T cell therapy may be at increased risk of TLS. Subjects should be closely monitored for TLS via laboratory assessments and symptoms from the start of LD chemotherapy until 28 days following CTX120 infusion.

Subjects at increased risk of TLS should receive prophylactic allopurinol (or a non-allopurinol alternative such as febuxostat) and increased oral/IV hydration during screening and before initiation of LD chemotherapy. Prophylaxis can be stopped after 28 days following CTX120 infusion or once the risk of TLS passes.

Sites should monitor and treat TLS as per their institutional standard of care, or according to published guidelines (Cairo and Bishop, 2004, Br J Haematol 127, 3-11). TLS management, including administration of rasburicase, should be instituted promptly when clinically indicated.

5.2.4 Cytokine Release Syndrome

CRS is due to hyperactivation of the immune system in response to CAR engagement of the target antigen, resulting in multi-cytokine elevation from rapid T cell stimulation and proliferation (Frey et al., 2014, Blood 124, 2296; Maude et al., 2014a, Cancer J 20, 119-122). When cytokines are released, a variety of clinical signs and symptoms associated with CRS may occur, including cardiac, gastrointestinal (GI), neurological, respiratory (dyspnea, hypoxia), skin, cardiovascular (hypotension, tachycardia), and constitutional (fever, rigors, sweating, anorexia, headaches, malaise, fatigue, arthralgia, nausea, and vomiting) symptoms, and laboratory (coagulation, renal, and hepatic) abnormalities.

The goal of CRS management is to prevent life-threatening sequelae while preserving the potential for the anticancer effects of CTX120. Symptoms usually occur 1 to 14 days after autologous CAR T cell therapy, but the timing of symptom onset has not been fully defined for allogeneic BCMA CAR T cells.

CRS should be identified and treated based on clinical presentation and not laboratory cytokine measurements. If CRS is suspected, grading and management should be performed as follows:
In Part A, grading and management should be performed according to the recommendations in Tables 10 and 12 below, which are adapted from the 2014 Lee criteria for CRS grading (Lee et al., 2014).
In Part B, grading should be applied according to the 2019 ASTCT (formerly known as American Society for Blood and Marrow Transplantation) consensus recommendations (Table 11 below) (Lee et al., 2019), and management should be performed according to the recommendations in Tables 10 and 12, which are adapted from published guidelines (Lee et al., 2014; Lee et al., 2019).

At the time of the original protocol version (V1.0), the established 2014 Lee criteria for CRS grading were applied (Lee et al., 2014). However, this has been updated to the ASTCT criteria (Lee et al., 2019), which have become the worldwide standard for CRS grading. Therefore, the ASTCT criteria is to be used during Part B (cohort expansion) of the trial. Both published CRS grading criteria (Lee et al., 2014; Lee et al., 2019) are to be used for future reporting of CRS.

Neurotoxicity is graded and managed as described in herein. End organ toxicity in the context of CRS management (Lee et al., 2019) refers only to hepatic and renal systems (as in the Penn Grading criteria) (Porter et al., 2018).

TABLE 10
Cytokine Release Syndrome Grading and Management Guidance, Part A

| CRS Severity¹ | Tocilizumab | Corticosteroids |
|---|---|---|
| Grade 1 | Tocilizumab may be considered in | N/A |
| Symptoms require symptomatic | consultation with the medical | |
| treatment only (e.g., fever, fatigue, | monitor. | |
| headache, myalgia, malaise). | | |
| Grade 2 | Administer tocilizumab 8 mg/kg [symbol] | Manage per grade 3 if no |
| Symptoms require and respond to | over 1 hour (not to exceed 800 mg) | improvement within 24 hours |
| moderate intervention. Oxygen | Repeat tocilizumab every 8 hours | after starting tocilizumab. |
| requirement <40% FiO₂ or | needed if not responsive to IV flui [symbol] | |
| hypotension responsive to fluids or | or increasing supplemental oxygen | |
| low dose of 1 vasopressor or grade | Limit to ≤3 doses in a 24-hour | |
| organ toxicity.² | period; maximum total of 4 doses. | |
| Grade 3 | Per grade 2. | If no improvement within 24 |
| Symptoms require and respond to | | hours after starting tocilizumab |
| aggressive intervention. Oxygen | | administer methylprednisolone |
| requirement ≥40% FiO₂ or | | l mg/kg IV twice daily. |
| hypotension requiring high-dose or | | Continue corticosteroids use |
| multiple vasopressors⁴ or grade 3 | | until the event is grade ≤1, then |
| organ toxicity or grade 4 | | taper over 3 days. |
| transaminitis. | | |
| Grade 4 | Per grade 2. | Per grade 3. |
| Life-threatening symptoms. | If no response to multiple doses of | |
| Requirements for ventilator support | tocilizumab and steroids, consider | |
| continuous veno-venous | using other anti-cytokine therapies | |
| hemodialysis or grade 4 organ | (e.g., siltuximab). | |
| toxicity (excluding transaminitis). | | |

CRS: cytokine release syndrome;
FiO2: fraction of inspired oxygen;
IV: intravenously;
N/A: not applicable.
¹See (Lee et al., 2014).
²Refer to 6.2.5 for management of neurologic toxicity. Organ toxicity refers to hepatic and renal systems only.
³Refer to tocilizumab prescribing information.
⁴See Table 13 for information on high-dose vasopressors. [symbol]
indicates data missing or illegible when filed

TABLE 11
ASTCT Cytokine Release Syndrome Grading Criteria, Part B

| CRS Parameter | Grade 1 | Grade 2 | Grade 3 | Grade 4 |
|---|---|---|---|---|
| Fever¹ | Temperature ≥38° C. | Temperature ≥38° [symbol] | Temperature ≥38° [symbol] | Temperature ≥38° [symbol] |
| With hypotension | None | Not requiring | Requiring a | Requiring multiple |
| | | vasopressors | vasopressor with or | vasopressors |
| | | | without vasopressin | (excluding |
| | | | | vasopressin) |
| And/or hypoxia² | None | Requiring low-flow | Requiring high-flov | Requiring positive |
| | | nasal cannula³ or | nasal cannula,³ | pressure (e.g., CPA |
| | | blow-by | facemask, | BiPAP, |
| | | | nonrebreather mask | intubation, and |
| | | | or Venturi mask | mechanical |
| | | | | ventilation) |

ASTCT: American Society for Transplantation and Cellular Therapy;
BiPAP: bilevel positive airway pressure;
C: Celsius;
CPAP: continuous positive airway pressure;
CRS: cytokine release syndrome;
CTCAE: Common Terminology Criteria for Adverse Events.
Note:
Organ toxicides associated with CRS may be graded according to CTCAE v5.0 but do not influence CRS grading.
¹Fever is defined as temperature ≥38° C. not attributable to any other cause. In subjects who have CRS then receive antipyretics or anticytokine therapy such as tocilizumab or steroids, fever is no longer required to grade subsequent CRS severity. In this case, CRS grading is driven by hypotension and/or hypoxia.
²CRS grade is determined by the more severe event: hypotension or hypoxia not attributable to any other cause. For example, a subject with temperature of 39.5° C., hypotension requiring 1 vasopressor, and hypoxia requiring low-flow nasal cannula is classified as grade 3 CRS.
³Low-flow nasal cannula is defined as oxygen delivered at ≤6 L/minute. Low-flow also includes blow-by oxygen delivery, sometimes used in pediatrics. High-flow nasal cannula is defined as oxygen delivered at >6 L/minute. [symbol]
indicates data missing or illegible when filed

TABLE 12
Cytokine Release Syndrome Grading and Management Guidance, Part B

| CRS Severity¹ | Tocilizumab | Corticosteroids |
|---|---|---|
| Grade 1 | Tocilizumab² may be considered in | N/A |
| | consultation with a medical practioner. | |
| Grade 2 | Administer tocilizumab 8 mg/kg IV over 1 hour | If no improvement within 24 hours |
| | (not to exceed 800 mg).² | after starting |
| | Repeat tocilizumab every 8 hours as needed | tocilizumab, administe [symbol] methylprednisolone |
| | if not responsive to IV fluids or increasing | 1 mg/kg IV twice daily. |
| | supplemental oxygen. | Continue corticosteroid |
| | Limit to ≤3 doses in a 24-hour period; | use until the event is grade ≤1, then |
| | maximum total of 4 doses. | taper over 3 days. |
| Grade 3 | Per grade 2. | Per grade 2. |
| Grade 4 | Per grade 2. | Per grade 2. |
| | If no response to multiple doses of | |
| | tocilizumab and steroids, consider using | |
| | other anticytokine therapies (e.g., | |
| | siltuximab, anakinra). | |

CRS: cytokine release syndrome;
IV: intravenously;
N/A: not applicable.
¹See Lee et al., 2019.
²Refer to tocilizumab prescribing information.
Throughout the duration of CRS, subjects should be provided with supportive care consisting of antipyretics, IV fluids, and oxygen. Subjects who experience grade ≥2 CRS (e.g., hypotension, not responsive to fluids, or hypoxia requiring supplemental oxygenation) should be monitored with continuous cardiac telemetry and pulse oximetry. For subjects experiencing grade 3 CRS, consider performing an echocardiogram to assess cardiac function. For grade 3 or 4 CRS, consider intensive care supportive therapy. Intubation for airway protection due to neurotoxicity (e.g., seizure) and not due to hypoxia should not be captured as grade 4 CRS. Similarly, prolonged intubation due to neurotoxicity without other signs of CRS (e.g., hypoxia) is not considered grade 4 CRS. The potential of an underlying infection in cases of severe CRS should be considered, as the presentation (fever, hypotension, hypoxia) is similar.
Resolution of CRS is defined as resolution of fever (temperature ≥38° C.), hypoxia, and hypotension (Lee et al., 2019). [symbol]
indicates data missing or illegible when filed

TABLE 13
High-dose Vasopressors

| Pressor | Dose* |
|---|---|
| Norepinephrine monotherapy | ≥20 μg/min |
| Dopamine monotherapy | ≥10 μg/kg/min |
| Phenylephrine monotherapy | ≥200 μg/min |
| Epinephrine monotherapy | ≥10 μg/min |
| If on vasopressin | Vasopressin + norepinephrine equivalent of ≥10 μg/min* |
| If on combination | Norepinephrine equivalent |
| vasopressors (not vasopressin) | of ≥20 μg/min** |

*All doses are required for ≥3 hours.
**VASST Trial vasopressor equivalent equation: norepinephrine equivalent dose = [norepinephrine (μg/min)] + [dopamine (μg/min)/2] + [epinephrine (μg/min)] + [phenylephrine (μg/min)/10]

5.2.5 Immune Effector Cell-Associated Neurotoxicity Syndrome (ICANS)

Neurotoxicity may occur at the time of CRS, during the resolution of CRS, or following resolution of CRS, and its pathophysiology is unclear. The recent ASTCT consensus recommendations further defined neurotoxicity associated with CRS as ICANS, a disorder characterized by a pathologic process involving the CNS following any immune therapy that results in activation or engagement of endogenous or infused T cells and/or other immune effector cells (Lee et al., 2019).

Signs and symptoms can be progressive and may include aphasia, altered level of consciousness, impairment of cognitive skills, motor weakness, seizures, and cerebral edema. ICANS grading (Table 15) was developed based on CAR T cell-therapy-associated TOXicity (CARTOX) working group criteria used previously in autologous CAR T cell trials (Neelapu et al., 2018). ICANS incorporates assessment of level of consciousness, presence/absence of seizures, motor findings, presence/absence of cerebral edema, and overall assessment of neurologic domains by using a modified tool called the ICE (immune effector cell-associated encephalopathy) assessment tool (see disclosures herein and Table 21).

Evaluation of any new onset neurotoxicity should include a neurological examination (including ICE assessment tool, Table 21), brain magnetic resonance imaging (MRI), and examination of the CSF (via lumbar puncture) as clinically indicated. Infectious etiology should be ruled out by performing a lumbar puncture whenever possible (especially for subjects with Grade 3 or 4 ICANS). If a brain MRI is not possible, all subjects should receive a noncontrast CT scan to rule out intracerebral hemorrhage. Electroencephalogram should also be considered as clinically indicated. Endotracheal intubation may be needed for airway protection in severe cases.

Nonsedating, antiseizure prophylaxis (e.g., levetiracetam) should be considered, especially in subjects with a history of seizures, for at least 21 days following CTX120 infusion or upon resolution of neurological symptoms (unless the antiseizure medication is considered to be contributing to the detrimental symptoms). Subjects who experience grade≥2 ICANS should be monitored with continuous cardiac telemetry and pulse oximetry. For severe or life-threatening neurologic toxicities, intensive care supportive therapy should be provided. Neurology consultation should always be considered. Monitor platelets and for signs of coagulopathy and transfuse blood products appropriately to diminish risk of intracerebral hemorrhage. Table 14 provides neurotoxicity grading and Table 15 provides management guidance.

For subjects who receive active steroid management for more than 3 days, antifungal and antiviral prophylaxis is recommended to mitigate a risk of severe infection with prolonged steroid use. Consideration for antimicrobial prophylaxis should also be given.

TABLE 14
ICANS Grading

| Neurotoxicity | | | | |
|---|---|---|---|---|
| Domain | Grade 1 | Grade 2 | Grade 3 | Grade 4 |
| ICE score ¹ | 7-9 | 3-6 | 0-2 | 0 (subject is unarousab [symbol] |
| | | | | and unable to undergo |
| | | | | ICE assessment) |
| Depressed level of | Awakens | Awakens | Awakens only to | Subject is unarousable |
| consciousness² | spontaneously | to voice | tactile stimulus | or requires vigorous or |
| | | | | repetitive tactile stimul [symbol] |
| | | | | to arise; stupor or coma [symbol] |
| Seizure | N/A | N/A | Any clinical seizure, | Life-threatening |
| | | | focal or generalized, | prolonged seizure (>5 |
| | | | that resolves rapidly | min) or repetitive |
| | | | or nonconvulsive | clinical or electrical |
| | | | seizures on EEG that | seizures without return |
| | | | resolve with | to baseline in between |
| | | | intervention | |
| Motor findings³ | N/A | N/A | N/A | Deep focal motor |
| | | | | weakness such as |
| | | | | hemiparesis or |
| | | | | paraparesis |
| Elevated ICP/ | N/A | N/A | Focal/local edema o [symbol] | Diffuse cerebral edema |
| cerebral edema | | | neuroimaging⁴ | on neuroimaging, |
| | | | | decerebrate or |
| | | | | decorticate posturing, |
| | | | | cranial nerve VI palsy, |
| | | | | papilledema, or |
| | | | | Cushing’s triad |

CTCAE: Common Terminology Criteria for Adverse Events;
EEG: electroencephalogram;
ICANS: immune effector cell-associated neurotoxicity syndrome;
ICE: immune effector cell-associated encephalopathy (assessment tool);
ICP: intracranial pressure;
N/A: not applicable.
Note:
ICANS grade is determined by the most severe event (ICE score, level of consciousness, seizure, motor findings, raised ICP/cerebral edema) not attributable to any other cause.
¹ A subject with an ICE score of 0 may be classified as grade 3 ICANS if awake with global aphasia, but a subject with an ICE score of 0 may be classified as grade 4 ICANS if unarousable (Table 21 for ICE assessment tool).
²Depressed level of consciousness should be attributable to no other cause (e.g., sedating medication).
³Tremors and myoclonus associated with immune effector therapies should be graded according to CTCAE v5.0 but do not influence ICANS grading.
⁴Intracranial hemorrhage with or without associated edema is not considered a neurotoxicity feature and is excluded from ICANS grading. It may be graded according to CTCAE v5.0. [symbol]
indicates data missing or illegible when filed

TABLE 15
ICANS Management Guidance

| Severity | Management |
|---|---|
| Grade 1 | Provide supportive care per institutional practice. |
| Grade 2 | Consider administering dexamethasone 10 mg IV every 6 hours (or equivalent |
| | methylprednisolone) unless subject already on equivalent dose of steroids for CRS. |
| | Continue dexamethasone use until event is grade ≤1, then taper over 3 days. |
| Grade 3 | Administer dexamethasone 10 mg IV every 6 hours, unless subject already on equivalent |
| | dose of steroids for CRS. |
| | Continue dexamethasone use until event is grade ≤1, then taper over 3 days. |
| Grade 4 | Administer methylprednisolone 1000 mg IV per day for 3 days; if improves, then manage |
| | as above. |

CRS: cytokine release syndrome;
ICANS: immune effector cell-associated neurotoxicity syndrome;
IV: intravenously.
Headache, which may occur in a setting of fever or after chemotherapy, is a nonspecific symptom. Headache alone may not necessarily be a manifestation of ICANS and further evaluation should be performed. Weakness or balance problem resulting from deconditioning and muscle loss are excluded from definition of ICANS. Similarly, intracranial hemorrhage with or without associated edema may occur due to coagulopathies in these subjects and are also excluded from definition of ICANS. These and other neurotoxicities should be captured in accordance with CTCAE v5.0.

6.2.6 Hemophagocytic Lymphohistiocytosis

Hemophagocytic lymphohistiocytosis is a clinical syndrome that is a result of an inflammatory response following infusion of CAR T cells in which cytokine production from activated T cells leads to excessive macrophage activation. Signs and symptoms of HLH may include fevers, cytopenias, hepatosplenomegaly, hepatic dysfunction with hyperbilirubinemia, coagulopathy with significantly decreased fibrinogen, and marked elevations in ferritin and C-reactive protein (CRP).

CRS and HLH may possess similar clinical syndromes with overlapping clinical features and pathophysiology. HLH may likely occur at the time of CRS or as CRS is resolving. HLH should be considered if there are unexplained elevated liver function tests or cytopenias with or without other evidence of CRS. Monitoring of CRP and ferritin may assist with diagnosis and define the clinical course.

If HLH is suspected:
Frequently monitor coagulation parameters, including fibrinogen. These tests may be done more frequently than indicated in the schedule of assessments, and frequency should be driven based on laboratory findings.
Fibrinogen should be maintained ≥100 mg/dL to decrease risk of bleeding.
Coagulopathy should be corrected with blood products.
Given the overlap with CRS, subjects should also be managed per CRS treatment guidance in Table 10 for Part A and Table 12 for Part B.

5.2.7 Cytopenias

Subjects receiving CTX120 are monitored for neutropenia (e.g., Grade 3) and/or thrombocytopenia, and appropriately supported. Monitor platelets and for signs of coagulopathy, and transfuse blood products appropriately to diminish risk of hemorrhage. Consideration should be given to antimicrobial and antifungal prophylaxis for any subject with prolonged neutropenia.

During dose escalation, G-CSF may be considered in cases of grade 3 or 4 neutropenia post-CTX120 infusion. During dose expansion G-CSF may be administered.

5.2.8 Graft Versus Host Disease

GvHD is seen in the setting of allogeneic SCT and is the result of immunocompetent donor T cells (the graft) recognizing the recipient (the host) as foreign. The subsequent immune response activates donor T cells to attack the recipient to eliminate foreign antigen-bearing cells. GvHD is divided into acute, chronic, and overlap syndromes based on both the time from allogeneic SCT and clinical manifestations. Signs of acute GvHD may include a maculopapular rash; hyperbilirubinemia with jaundice due to damage to the small bile ducts, leading to cholestasis; nausea, vomiting, and anorexia; and watery or bloody diarrhea and cramping abdominal pain (Zeiser and Blazar, 2017; N Engl J Med 377, 2167-2179). To support the proposed clinical study, a 12-week nonclinical Good Laboratory Practice-compliant GvHD and tolerability study was performed in immunocompromised mice treated with a single IV dose of 4×10⁷ CTX120 cells per mouse (approximately 1.6×10⁹ cells/kg). This dose level exceeds the proposed highest clinical dose by more than 100-fold when normalized for body weight. CTX120 did not induce clinical GvHD in immunocompromised (NSG) mice during the course of the 12-week study.

Further, due to the specificity of CAR insertion at the TRAC locus, it is highly unlikely for a T cell to be both CAR⁺ and TCR⁺. Remaining TCR⁺ cells are removed during the manufacturing process by immunoaffinity chromatography on an anti-TCR antibody column to achieve <0.5% TCR⁺ cells in the final product. A dose limit of 7×10⁴ TCR⁺ cells/kg may be imposed for all dose levels. This limit is lower than the limit of 1×10⁵ TCR⁺ cells/kg based on published reports on the number of allogeneic cells capable of causing severe GvHD during SCT with haploidentical donors (Bertaina et al., 2014, Blood 124, 822-826). Subjects should be monitored closely for signs of acute GvHD following infusion of CTX120.

Diagnosis and grading of GvHD should be based on the published MAGIC criteria (Harris et al., 2016), as outlined in Table 16 below.

TABLE 16
Criteria for Grading Acute GvHD

| | Skin | | | | Lower GI |
|---|---|---|---|---|---|
| | (active erythema | Liver | | | (stool |
| Stage | only) | (bilirubin) | | Upper GI | output/day) |
| 0 | No active | <2 | mg/dL | No or intermittent | <500 mL/day or |
| | (erythematous) | | | nausea, vomiting, or | <3 episodes/day |
| | GvHD rash | | | anorexia | |
| 1 | Maculopapular rash | 2-3 | mg/dL | Persistent nausea, | 500-999 mL/day or |
| | <25% BSA | | | vomiting, or anorexia | 3-4 episodes/day |
| 2 | Maculopapular rash | 3.1-6 | mg/dL | — | 1000-1500 mL/day or |
| | 25-50% BSA | | | | 5-7 episodes/day |
| 3 | Maculopapular rash | 6.1-15 | mg/dL | — | >1500 mL/day or |
| | >50% BSA | | | | >7 episodes/day |
| 4 | Generalized | >15 | mg/dL | — | Severe abdominal pain |
| | erythroderma | | | | with or without ileus, o [symbol] |
| | (>50% | | | | grossly bloody stool |
| | BSA) plus bullous | | | | (regardless of stool |
| | formation and | | | | volume) |
| | desquamation >5% | | | | |
| | BSA | | | | |

BSA: body surface area;
GI: gastrointestinal;
GvHD: graft versus host disease.
Overall GvHD grade may be determined based on most severe target organ involvement.
Grade 0: No stage 1-4 of any organ
Grade 1: Stage 1-2 skin without liver, upper GI, or lower GI involvement
Grade 2: Stage 3 rash and/or stage 1 liver and/or stage 1 upper GI and/or stage 1 lower GI
Grade 3: Stage 2-3 liver and/or stage 2-3 lower GI, with stage 0-3 skin and/or stage 0-1 upper GI
Grade 4: Stage 4 skin, liver, or lower GI involvement, with stage 0-1 upper GI [symbol]
indicates data missing or illegible when filed

Potential confounding factors that may mimic GvHD such as infections and reactions to medications should be ruled out. Skin and/or GI biopsy should be obtained for confirmation before or soon after treatment has been initiated. In instance of liver involvement, liver biopsy should be attempted if clinically feasible. Sample(s) of all biopsies may also be sent to a central laboratory for pathology assessment. Details of sample preparation and shipment are contained in the Laboratory Manual.

Recommendations for management of acute GvHD are outlined in Table 17 below. To allow for intersubject comparability at the end of the trial, these recommendations should be followed except in specific clinical scenarios in which following them could put the subject at risk.

TABLE 17
Acute GvHD Management

| Grade | Management |
|---|---|
| 1 | Skin: Topical steroids or immunosuppressants; if stage 2: prednisone 1 mg/kg (or equivalent |
| | dose) |
| 2-4 | Initiate prednisone 2 mg/kg daily (or equivalent dose). |
| | IV form of steroid such as methylprednisolone should be considered if there are concerns |
| | with malabsorption. |
| | Steroid taper may begin after improvement is seen after ≥3 days of steroids. Taper should b [symbol] |
| | 50% decrease of total daily steroid dose every 5 days. |
| | GI: In addition to steroids, start anti-diarrheal agents as per standard practice. |

GI: gastrointestinal;
IV: intravenous. [symbol]
indicates data missing or illegible when filed

Decisions to initiate second-line therapy should be made sooner for subjects with more severe GvHD. For example, secondary therapy may be indicated after 3 days with progressive manifestations of GvHD, after 1 week with persistent grade 3 GvHD, or after 2 weeks with persistent grade 2 GvHD. Second-line systemic therapy may be indicated earlier in subjects who cannot tolerate high-dose glucocorticoid treatment (Martin et al., 2012, Biol Blood Marrow Transplant 18, 1150-1163). Choice of secondary therapy and when to initiate are based on medical practitioner's judgment.

Management of refractory acute GvHD or chronic GvHD is per institutional guidelines. Anti-infective prophylaxis measures should be instituted per local guidelines when treating subjects with immunosuppressive agents (including steroids).

5.2.9 Hypotension and Renal Insufficiency

Hypotension and renal insufficiency is to be monitored in subjects receiving CTX120 cells and should be treated with IV administration of normal saline boluses according to institutional practice guidelines. Dialysis should be considered when appropriate.

### 6. Study Procedures

Both the dose escalation and expansion parts of the study consist of 3 distinct stages: (1) screening and eligibility confirmation,
(2) treatment with various LD/immunomodulatory agents and CTX120 infusion, and (3) follow-up.

During the screening period, subjects are assessed according to the eligibility criteria outlined above. After enrollment, subjects receive various regimens of LD/immunomodulatory agents, followed by CTX120 infusion. After completing the treatment period, subjects are assessed for MM response, disease progression, and survival. Throughout all study periods, subjects are regularly monitored for safety.

A complete schedule of assessments is provided in Table 18 and Table 19 below. Descriptions of all required study procedures are provided in this section. In addition to protocol-mandated assessments, subjects should be followed per institutional guidelines, and unscheduled assessments should be performed when clinically indicated.

Certain assessments for visits after Day 8 may be performed as in-home or alternate-site visits. Assessments include hospital utilization, changes in health and/or changes in medications, body system assessment, vital signs, weight, PRO questionnaire distribution, and blood sample collections for local and central laboratory assessments.

For the purposes of this protocol, there is no Day 0. All visit dates and windows are to be calculated using Day 1 as the date of CTX120 infusion.

TABLE 18
Schedule of Assessments: Screening, Treatment, and Primary Follow-up (Screening to Month 24)

| | | Treatment | | | | | | | | | | |
|---|---|---|---|---|---|---|---|---|---|---|---|---|
| | | (Stage 2) | | | | | | | | | | |
| | | D-14 | D-5 | | Follow-up (Stage 3) | | | | | | | |
| Study Stage | Screening¹ | to | to D | | | D3 + | D5 ± | D8 ± | D10 ± | D14 ± | D21 ± | D28 ± |
| Day | (Stage 1) | D-6 ⁽²⁾ | 3 | D1⁽³⁾ | D2 | 1d | 2d | 2d | 2d | 2d | 2d | 4d |
| Informed | | | | | | | | | | | | |
| consent [symbol] | | | | | | | | | | | | |
| Medical history | | | | | | | | | | | | |
| Physical exam | | X | X | X | X | | | X | X | X | X | X ⁵ |
| Vital signs ⁶ | | X | X | X | X | | | X | X | X | X | X ⁵ |
| Height, weight | | X | X | X | | | | X | | X | | X |
| Pregnancy test | | X | X | | | | | | | | | |
| ECOG status | | | | X | | | | | | | | X |
| Echo- | | | | | | | | | | | | |
| cardiogram [symbol] | | | | | | | | | | | | |
| 12-lead ECG ¹⁰ | | X | X | X | | | | | | | | X |
| ICE | | | | X | X | | | X | | | | X |
| assessment ¹¹ | | | | | | | | | | | | |
| PRO ¹² | | | | | | | | | | | | X |

| Concomitant | Continuous | |
|---|---|---|
| meds ¹³ | | |
| Adverse events | Continuous | |
| Hospital | | Continuous |
| utilization | | |

Treatment

| | | |
|---|---|---|
| LD | X | |
| chemotherapy [symbol] | | |
| CTX120 | | X |
| infusion ¹⁸ | | |

MM Disease/Response Assessments (Central)

| | | |
|---|---|---|
| Serum/urine | | X |
| biochemistry ¹⁹ | | |
| Whole body | | X |
| PET/CT scan ²⁰ | | |
| BM | X ²² | X |
| aspirate/biopsy [symbol] | | |

Laboratory Assessments (Local) ⁸

| | | | | | | | | | |
|---|---|---|---|---|---|---|---|---|---|
| CBC w/ | X | X | X | X | X | X | X | X | X²⁴ |
| differential | | | | | | | | | |
| 23, 24 | | | | | | | | | |
| Serum | X | X | X | X | X | X | X | X | X⁵ |
| chemistry [symbol] | | | | | | | | | |
| Coagulation | X | X | X | X | X | X | X | | X |
| parameters | | | | | | | | | |
| Viral serology ²⁵ | | | | | | | | | |
| Immunoglobulin | | | X | | | | | | X |
| Ferritin, CRP | | | X | X | X | X | X | X | X |
| Lymphocyte | X | X | X | | X | | X | X | X |
| subsets ²⁶ | | | | | | | | | |
| Serum B2M ²⁷ | | | | | | | | | |
| BM cytogenetics ²⁷ | | | | | | | | | |

Biomarkers (Blood, Central)

| CTX120 | | | | X²⁹ | X | | | X | X | X | X | X |
|---|---|---|---|---|---|---|---|---|---|---|---|---|
| levels ² [symbol] | | | | pre/p | | | | | | | | |
| Cytokines ³⁰ | | | | X | X | | | X | X | X | X | X |
| Anti-CTX120 ³¹ | | | | | | | | | | | | X |
| DNA | | | | | | | | | | | | |
| Cell-free DNA | | | | | | | | | | | | X |
| Exploratory | | | X ³³ | X | X | | | X | X | X | X | X |
| biomarkers ³⁴ | | | | | | | | | | | | |
| | | Follow-up (Stage 3) | | | | | | | | | | |
| Study Stage | Screening¹ | M2 ± | M3 ± | M4 ± | M5 ± | M6 ± | M9 ± | M12 ± | M15 ± | M18 ± | M21 ± | M24 ± |
| Day | (Stage 1) | 7d | 7d | 7d | 7d | 14d | 14d | 14d | 14d | 14d | 14d | 21d |
| Informed | | | | | | | | | | | | |
| consent [symbol] | | | | | | | | | | | | |
| Medical history | | | | | | | | | | | | |
| Physical exam | | X ⁵ | X ⁵ | X ⁵ | X ⁵ | | X | X | X | X | X | X |
| Vital signs ⁶ | | X ⁵ | X ⁵ | X ⁵ | X ⁵ | | X | X | X | X | X | X |
| Height, weight | | X ⁷ | X ⁷ | X ⁷ | X ⁷ | | | | | | | |
| Pregnancy test | | | | | | | | | | | | |
| ECOG status | | | X | | | | | | | | | |
| Echo- | | | | | | | | | | | | |
| cardiogram [symbol] | | | | | | | | | | | | |
| 12-lead ECG ¹⁰ | | | | | | | | | | | | |
| ICE | | | | | | | | | | | | |
| assessment ¹¹ | | | | | | | | | | | | |
| PRO ¹² | | | X | | | | | X | | | | X |
| Concomitant | | Continuous | | | | | | | | | | |
| meds ¹³ | | | | | | | | | | | | |
| Adverse events | | Continuous | | | | | | | | | | |
| Hospital | | Continuous | | | | | | | | | | |
| utilization | | | | | | | | | | | | |
| Treatment | | | | | | | | | | | | |
| LD | | | | | | | | | | | | |
| chemotherapy [symbol] | | | | | | | | | | | | |
| CTX120 | | | | | | | | | | | | |
| infusion ¹⁸ | | | | | | | | | | | | |
| MM Disease/Response Assessments (Central) | | | | | | | | | | | | |
| Serum/urine | | X | X | X | X | | X | X | X | X | X | X |
| biochemistry ¹⁹ | | | | | | | | | | | | |
| Whole body | | | X | | | | X | X | X | X | X | X |
| PET/CT scan ²⁰ | | | | | | | | | | | | |
| BM | | | X | | | | | X | | | | |
| aspirate/biopsy [symbol] | | | | | | | | | | | | |
| Laboratory Assessments (Local) ⁸ | | | | | | | | | | | | |
| CBC w/ | | | X | X | X | | | X | X | X | X | X |
| differential | | | | | | | | | | | | |
| 23, 24 | | | | | | | | | | | | |
| Serum | | X [symbol] | X ⁵ | X⁵ | X ⁵ | | | X | X | X | X | X |
| chemistry [symbol] | | | | | | | | | | | | |
| Coagulation | | | | | | | | | | | | |
| parameters | | | | | | | | | | | | |
| Viral serology ²⁵ | | | | | | | | | | | | |
| Immunoglobulin | | | X | X | X | | | X | X | X | X | X |
| Ferritin, CRP | | | | X | X | | | | | | | |
| Lymphocyte | | | X | X | X | | | X | | | | |
| subsets ²⁶ | | | | | | | | | | | | |
| Serum B2M ²⁷ | | | | | | | | | | | | |
| BM cytogenetics ²⁷ | | | | | | | | | | | | |
| Biomarkers (Blood, Central) | | | | | | | | | | | | |
| CTX120 | | | X | X | X | | | X | X | X | X | X |
| levels ² [symbol] | | | | | | | | | | | | |
| Cytokines ³⁰ | | | | | | | | | | | | |
| Anti-CTX120 ³¹ | | | X | | | | | X | | | | X |
| DNA | | | | | | | | | | | | |
| Cell-free DNA | | | X | | | | | X | | X | X | X |
| Exploratory | | | X | X | X | | | X | X | X | X | X |
| biomarkers ³⁴ | | | | | | | | | | | | |

Ab: antibody;
AE: adverse event;
AESI: adverse event of special interest;
ANC: absolute neutrophil count;
B2M: beta-2 microglobulin;
BM: bone marrow;
CBC: complete blood count;
chemo: chemotherapy;
CNS: central nervous system;
con meds: concomitant medications;
CR: complete response;
CRP: C-reactive protein;
CRS: cytokine release syndrome;
CT: computed tomography;
D or d: day;
DL: Dose Level;
ECG: electrocardiogram;
ECOG: Eastern Cooperative Oncology Group;
EORTC QLQ-C30 and QLQ-MY20: European Organisation for Research and Treatment of Cancer QLQ-C30 and QLQ-MY20 questionnaires;
FLC: free light chain;
HBV: hepatitis B virus;
HCV: hepatitis C virus;
HIV: human immunodeficiency virus;
ICE: immune effector cell-associated encephalopathy;
IMWG: International Myeloma Working Group;
IV: intravenously;
LD: lymphodepleting;
M: month;
M-protein: monoclonal protein;
MM: multiple myeloma;
MRI: magnetic resonance imaging;
PCR: polymerase chain reaction;
PET: positron emission tomography;
PK: pharmacokinetics;
PRO: patient-reported outcome;
SAE: serious adverse event;
SPEP: serum M-protein quantitation by electrophoresis;
TBNK: T, B, and natural killer cells;
UPEP: urine M-protein quantitation by electrophoresis.
NOTE:
Certain assessments for visits after Day 8 may be performed as in-home or alternate-site visits. Assessments include hospital utilization, changes in health and/or changes in medications, body system assessment, vital signs, weight, PRO questionnaire distribution, and blood sample collections for local and central laboratory assessments.
¹Screening assessments to be completed within 14 days of informed consent. Subjects may be allowed a one-time rescreening, which may take place within 3 months of initial consent.
³All baseline assessments on Day 1 are to be performed prior to CTX120 infusion unless otherwise specified; refer to Laboratory Manual for details.
⁴ Includes complete surgical and cardiac history.
⁶ Includes sitting blood pressure, heart rate, respiratory rate, pulse oximetry, and temperature.
⁷ Height at screening only.
⁸ Assessed at local laboratory. Refer to Table 25 for hematology and serum chemistry assessments.
⁹ For female subjects of childbearing potential. Serum pregnancy test conducted at screening. Serum or urine pregnancy test conducted within 72 hours before start of LD chemotherapy.
¹⁰ Prior to LD chemotherapy, and prior to CTX120 infusion.
¹¹ On Day 1 prior to CTX120 administration. If CNS symptoms persist, ICE assessment (Table 22) should continue to be performed approximately every 2 days until symptom resolution to grade 1 or baseline.
¹² EORTC QLQ-C30 and QLQ-MY20, and EuroQol EQ-5D-5L questionnaires.
¹³ All concomitant medications to be collected up to 3 months post-CTX120 infusion, then only select concomitant medications may be collected.
¹⁴ Collect all AEs from informed consent to Month 3 visit, collect all SAEs and AESIs after Month 3 visit to Month 60 visit. All SAEs and AESIs should be reported for up to 3 months after CTX120 infusion if a subject starts a new anticancer therapy before Month 3 study visit. If a subject starts a new anticancer therapy after Month 3 study visit, only CTX120-related SAEs and AESIs should be reported.
¹⁶ Start LD chemotherapy within 7 days of study enrollment (i.e., confirmation of eligibility). After completion of LD chemotherapy, ensure washout period of ≥ 48 hours (but ≤ 7 days) before CTX120 infusion. Physical exam, weight, and coagulation laboratories performed prior to first dose of LD chemotherapy. Vital signs, CBC, clinical chemistry, and AEs/concomitant medications assessed and recorded daily (i.e., 3 times) during LD chemotherapy.
¹⁸ CTX120 administered 48 hours to 7 days after completion of LD chemotherapy. Start dose escalation at DL1 for Cohort A and at DL2 or DL3 for Cohort B.
¹⁹ Central lab testing and review of M-protein measurements in serum and urine: SPEP, serum immunofixation, serum FLC (kappa and lambda), 24-h UPEP, urine immunofixation, and quantitative immunoglobulins. Note: Screening to determine eligibility does NOT require central review, but samples for baseline disease assessment should be collected for central lab testing. Note: Screening 24-hour urine collection may begin the day before informed consent.
²⁰ Baseline whole body (vertex to toes) PET/CT to be performed at screening (i.e., within 28 days prior to CTX120 infusion) and upon suspected CR. For subjects with evidence of extramedullary disease (e.g., extramedullary plasmacytoma or myelomatous lesion with soft tissue involvement), postinfusion scans may be conducted per the schedule of assessments, per IMWG response criteria, and as clinically indicated. In subjects with extramedullary disease, the CT portion of PET/CT should be of diagnostic quality (e.g., CT with IV contrast) sufficient for tumor size measurement. MRI with contrast may be used for the CT portion when CT is clinically contraindicated or as required by local regulation.
²¹ BM biopsy to confirm CR (by immunohistochemistry, central testing) as part of disease evaluation. BM biopsy should be performed at time of disease relapse whenever clinically feasible. For any BM aspirates collected, samples should be sent for CTX120 levels and/or other exploratory analyses. BM sample collection (aspirate and biopsy) at screening should be performed during the 14-day screening period. All other bone marrow sample collection should be performed ± 5 days of visit date.
²² Optional for subjects who consent. If performed, serum and urine MM response assessments should also be performed for this time point.
23 In case of grade 3 or 4 neutropenia and thrombocytopenia, collect samples weekly until resolution to grade ≤ 2.
²⁵ Infectious disease testing (HIV-1, HIV-2, HCV antibody and PCR, HBV surface antigen, HBV surface antibody, HBV core antibody) performed within 30 days of providing informed consent may be considered for subject eligibility.
²⁶ Lymphocyte subset assessment at screening before first day of LD chemotherapy, before CTX120 infusion, and all listed time points may be assessed at local laboratory. To include 6-color TBNK panel, or equivalent for T, B, and natural killer cells (see Laboratory Manual).
²⁷ Serum B2M and cytogenetics (bone marrow) at screening only and assessed locally.
²⁸ If CRS occurs, samples for assessment of CTX120 levels may be collected every 48 hours between scheduled visits until CRS resolves. Continue sample collection for all listed time points.
²⁹ Two samples are to be collected on Day 1: one before CTX120 infusion and another 20 (±5) min after the end of CTX120 infusion.
³⁰ Additional cytokine samples should be collected daily for the duration of CRS.
³¹ Continue sample collection for all listed time points.
³³ Prior to first day of LD chemotherapy only.
³⁴ Samples for exploratory biomarkers should also be sent from any lumbar puncture. BM sample collection (aspirate/biopsy), or suspected GvHD tissue biopsy performed following CTX120 infusion. If CRS occurs, collect samples for exploratory biomarker assessment every 48 hours between scheduled visits until CRS resolves. [symbol]
indicates data missing or illegible when filed

TABLE 19
Schedule of Assessments: Progressive Disease and Secondary Follow-up (Months 30-60)

| | M30 | M36 | M42 | M48 | M54 | M60 | Progressive | Secondary |
|---|---|---|---|---|---|---|---|---|
| Assessments | (±21d) | (±21d) | (±21d) | (±21d) | (±21d) | (±21d) | Disease ¹ | Follow-Up ² |
| Physical exam | | | X | | | | | X |
| Vital signs ³ | | | X | | | | | X |
| PRO ⁴ | | | | | | | | |
| Concomitant medications ⁵ | | | X | | | | | X |
| AEs ⁶ | | | X | | | | | X |
| MM disease/response assessment ⁷ | | | X | | | | | |
| CBC with differential ⁸ | | | X | | | | | X |
| Serum chemistry ⁸ | | | X | | | | | X |
| Immunoglobulins ^{8,9} | | | X | | | | | |
| CTX120 levels (blood, central) ^{9,10} | | | X | | | | | X |
| Anti-CTX120 (blood, central) ⁹ | | | | | | | | |
| Lymphocyte subsets ⁸ | | | X | | | | | |
| Exploratory biomarkers | | | X | | | | | X |
| (blood, central) ¹¹ | | | | | | | | |

Ab: antibody;
AE: adverse event;
BM: bone marrow;
CBC: complete blood count;
CT: computed tomography;
d: days;
EORTC QLQ-C30 and QLQ-MY20: European Organisation for Research and Treatment of Cancer QLQ-C30 and QLQ-MY20 questionnaires;
IMWG: International Myeloma Working Group;
M: month;
M-protein: monoclonal protein;
MM: multiple myeloma;
PD: progressive disease;
PET: positron emission tomography;
PRO: patient-reported outcome;
SAE: serious adverse event;
SCT: stem cell transplant.
NOTE:
Certain assessments for visits after Day 8 may be performed as in-home or alternate-site visits. Assessments include hospital utilization, changes in health and/or changes in medications, body system assessment, vital signs, weight, PRO questionnaire distribution, blood sample collections for local and central laboratory assessments.
¹ Subjects with PD may discontinue the normal schedule of assessments, undergo study assessments listed, then secondary follow-up (see footnote 2).
² Subjects with PD or who partially withdraw consent may discontinue the normal schedule of assessments, attend annual study visits, and undergo secondary follow-up consisting of these procedures at a minimum: abbreviated physical exam, CBC with differential, serum chemistry, disease assessment/survival status, CTX120 persistence, select concomitant medications/procedures (anticancer therapy, disease-related surgery, SCT), and select AEs (treatment-related AEs and SAEs, new malignancies, new/worsening autoimmune, immune deficiency, or neurological disorders).
3 Includes temperature, blood pressure, pulse rate, and respiratory rate.
⁴ EORTC QLQ-C30, QLQ-MY20, and EuroQol EQ-5D-5L questionnaires.
⁵ Only select concomitant medications may be collected.
⁶ Collect all AEs from informed consent to Month 3 visit, collect all SAEs and AESIs after Month 3 visit to Month 60 visit. All SAEs and AESIs should be reported for up to 3 months after CTX120 infusion if a subject starts a new anticancer therapy before Month 3 study visit. If a subject starts a new anticancer therapy after the Month 3 study visit, only CTX120-related SAEs and AESIs should be reported.
⁷ Disease evaluations based on assessments in accordance with IMWG response criteria (Kumar et al., 2016) and may include serum and urine M-protein measurements, and, if deemed appropriate, whole body PET/CT and BM aspirate and biopsy as clinically indicated.
⁸ Assessed at local laboratory. To include 6-color TBNK panel, or equivalent for T, B, and NK cells (see Lab Manual for additional details).
⁹ Continue sample collection for all listed time points.
¹⁰ In addition to time points listed, samples for analysis of CTX120 levels and/or exploratory analyses should be sent to the central laboratory from any unscheduled collection of blood, BM aspirate, or biopsy of extramedullary plasmacytoma.
¹¹ Samples for exploratory biomarkers should be sent from any lumbar puncture. BM sample collection (aspirate/biopsy), or suspected GvHD tissue biopsy performed following

6.1 Subject Screening and Enrollment

The screening period begins on the date that the subject signs the ICF and continues through confirmation of eligibility and enrollment into the study. Once informed consent has been obtained, the subject may be screened to confirm study eligibility as outlined in the schedule of assessments (Table XXX). Screening assessments should be completed within 14 days of a subject signing the informed consent.

Subjects are allowed a one-time rescreening, which may take place within 3 months of the initial consent.

6.2 Study Assessments

Refer to the schedule of assessments (Table 18 and Table 19) for the timing of the required procedures. Demographic data, including age, sex, race, and ethnicity, are collected. Medical history, including a full history of the subject's disease, previous cancer treatments, and response to treatment from date of diagnosis are obtained. Cardiac, neurological, and surgical history are obtained. For trial entry, all subjects must fulfill all inclusion criteria and have none of the exclusion criteria as described herein.

6.2.1. Physical Examination

Physical examination, including examination of major body systems, including general appearance, skin, neck, head, eyes, ears, nose, throat, heart, lungs, abdomen, lymph nodes, extremities, and nervous system, are performed at every study visit and the results documented. Changes noted from the exam performed at screening are recorded as an AE.

Vital signs are recorded at every study visit and include sitting blood pressure, heart rate, respiratory rate, pulse oximetry, and temperature. Weight are obtained according to the schedule in Table 18, and height is only to be obtained at screening.

6.2.2 ECOG Performance Status

Performance status is assessed at the screening, CTX120 infusion (Day 1, prior to infusion), Day 28, and Month 3 visits using the ECOG scale to determine the subject's general well-being and ability to perform activities of daily life (Table 20 below).

TABLE 20
ECOG Performance Status Scale

| Grade | Description |
|---|---|
| 0 | Fully active, able to carry on all pre-disease performance without restriction |
| 1 | Restricted in physically strenuous activity but ambulatory and able to carry out work |
| | of a light or sedentary nature, e.g., light house work, office work |
| 2 | Ambulatory and capable of all self-care but unable to carry out any work activities; u [symbol] |
| | and about more than 50% of waking hours |
| 3 | Capable of only limited self-care; confined to bed or chair more than 50% of waking |
| | hours |
| 4 | Completely disabled; cannot carry on any self-care; totally confined to bed or chair |
| 5 | Dead |

[symbol]

indicates data missing or illegible when filed

### Developed by the Eastern Cooperative Oncology Group, Robert L. Comis, MD, Group Chair (Oken et al., 1982, Am J Clin Oncol 5, 649-655).

6.2.3. Echocardiogram

A transthoracic cardiac echocardiogram (for assessment of left ventricular ejection fraction) may be performed and read by trained medical personnel at screening to confirm eligibility.

Additional cardiac assessments should be performed during grade 3 or 4 CRS for all subjects who require >1 fluid bolus for hypotension, who are transferred to the intensive care unit for hemodynamic management, or who require any dose of vasopressor for hypotension (Brudno and Kochenderfer, 2016, Blood 127, 3321-3330).

6.2.4 Electrocardiogram

Twelve (12)-lead electrocardiograms (ECGs) are obtained during screening, prior to LD chemotherapy on the first day of treatment, prior to CTX120 administration on Day 1, and on Day 28. QTc and QRS intervals are determined from ECGs. Additional ECGs may be obtained.

6.2.5 Immune Effector Cell-Associated Encephalopathy Assessment

Neurocognitive assessment may be performed using ICE assessment. The ICE assessment tool is a slightly modified version of the CARTOX-10 screening tool, which now includes a test for receptive aphasia (Neelapu et al., 2018, N Engl J Med 377, 2531-2544). ICE assessment examines various areas of cognitive function: orientation, naming, following commands, writing, and attention (Table 21).

TABLE 21
ICE Assessment

| Domain | Assessment | Maximum Score |
|---|---|---|
| Orientation | Orientation to year, month, city, hospital | 4 points |
| Naming | Name 3 objects (e.g., point to clock, pen, button) | 3 points |
| Following | Ability to follow commands | 1 point |
| command | (e.g., “Show me 2 fingers” or “Close | |
| | your eyes and stick out your tongue”) | |
| Writing | Ability to write a standard sentence (includes a noun and verb) | 1 point |
| Attention | Ability to count backward from 100 by 10 | 1 point |

ICE score may be reported as the total number of points (0-10) across all assessments.

ICE assessment may be performed at screening, before administration of CTX120 on Day 1, and on Days 2, 3, 5, 8, and 28. If a subject experiences CNS symptoms, ICE assessment should continue to be performed approximately every 2 days until resolution of symptoms to grade 1 or baseline. To minimize variability, whenever possible the assessment should be performed by the same research staff member who is familiar with or trained in administration of the ICE assessment tool.

6.2.6. Patient-Reported Outcomes

Three patient-reported outcome (PRO) surveys, the European Organisation for Research and Treatment of Cancer (EORTC) QLQ-C30, EORTC QLQ-MY20, and the EuroQol EQ-5D-5L questionnaires, are administered according to the schedule in Table 18 and Table 19. Questionnaires should be completed (self-administered in the language the subject is most familiar) before clinical assessments are performed.

The EORTC QLQ-C30 is a questionnaire designed to measure cancer patients' physical, psychological, and social functions. It is composed of 5 multi-item scales (physical, role, social, emotional, and cognitive function) and 9 single items (pain, fatigue, financial impact, appetite loss, nausea/vomiting, diarrhea, constipation, sleep disturbance, and quality of life). The EORTC QLQ-C30 is validated and has been widely used among cancer patients, including in multiple myeloma patients (Wisloff et al., 1996, Br J Haematol 92, 604-613; and Wisloff and Hjorth, 1997, Br J Haematol 97, 29-37).

The QLQ-MY20 questionnaire is the myeloma-specific module of EORTC QLQ-C30, designed for patients with MM to assess the symptoms and side effects of treatment and their impact on everyday life. The module comprises 20 questions addressing 4 domains of quality of life important in myeloma: pain, treatment side effects, social support and future perspective, disease-specific symptoms and their impact on everyday life, treatment side effects, social support, and future perspective (Cocks et al., 2007, Eur J Cancer 43, 1670-1678).

The EQ-5D-5L is a generic measure of health status and contains a questionnaire that assesses 5 domains, including mobility, self-care, usual activities, pain/discomfort, and anxiety/depression, plus a visual analog scale. EQ-5D-5L has been used in conjunction with QLQ-C30 and QLQ-MY20 in MM (Moreau et al., 2019, Leukemia 33, 2934-2946).

6.2.7 MM Disease and Response Assessments

Disease evaluations may be based on assessments in accordance with the IMWG criteria for response and minimal residual disease (MRD) assessment in multiple myeloma (Table 22 below) (Kumar et al., 2016) and is to be assessed. Determination of study eligibility and decisions regarding subject management and disease progression is to be made. For efficacy analyses, disease outcome is graded using IMWG response criteria provided in Table 22 below. MM disease and response evaluation should be conducted per the schedule in Table 12 and Table 13, and may include the assessments described below. All response categories (including progression) require 2 consecutive assessments made at least 1 week apart at any time before the institution of any new therapy.

TABLE 22
Standard IMWG Response Criteria

| Response¹ | Description |
|---|---|
| Stringent Complete | Complete response as defined below |
| Response (sCR) | plus normal FLC ratio² and absence of |
| | clonal cells in BM biopsy by immunohistochemistry (κ/λ ratio ≤4:1 or ≥1:2 |
| | for κ and λ patients, respectively, after counting ≥100 plasma cells).³ |
| Complete Response (CR) | Negative immunofixation on the serum and urine and disappearance of any |
| | soft tissue plasmacytomas and <5% plasma cells in BM aspirates.⁴ |
| Very Good Partial | Serum and urine M-protein detectable |
| Response (VGPR) | by immunofixation but not on |
| | electrophoresis or ≥90% reduction in serum M-protein plus urine M-protein |
| | level <100 mg per 24 h. |
| Partial Response | ≥50% reduction of serum M-protein plus |
| (PR) | reduction in 24 h urinary M-protein by |
| | ≥90% or to <200 mg per 24 h; |
| | If the serum and urine M-protein are unmeasurable, ≥50% decrease in the |
| | difference between involved and uninvolved FLC levels is required in place |
| | of the M-protein criteria; |
| | If serum and urine M-protein are unmeasurable, and serum-free light assay |
| | also unmeasurable, ≥50% reduction in plasma cells is required in place of M- |
| | protein, provided baseline BM plasma-cell percentage was ≥30%. In additi [symbol] |
| | to these criteria, if present at baseline, ≥50% reduction in the size (SPD)⁵ of |
| | soft tissue plasmacytomas is required. |
| Minimal Response (MR) | ≥25% but ≤49% reduction of serum M-protein and reduction in 24-h urine |
| | M- protein by 50-89%. In addition to the above listed criteria, if present at |
| | baseline, |
| | ≥50% reduction in the size (SPD)⁵ of soft tissue plasmacytomas is also |
| | required. |
| Stable Disease (SD) | Not recommended for use as an indicator of response; stability of disease is |
| | best described by providing the time-to- progression estimates. Not meeting |
| | criteria for CR, VGPR, PR, MR, or PD. |
| Progressive | Any one or more of the following criteria: |
| Disease (PD) [symbol] | Increase of 25% from lowest confirmed response value in ≥1 of the followi [symbol] |
| | criteria: |
| | Serum M-protein (absolute increase must be ≥0.5 g/dL); |
| | Serum M-protein increase ≥1 g/dL, if the lowest M component was ≥5 g/dL |
| | Urine M-protein (absolute increase must be ≥200 mg/24 h); |
| | In patients without measurable serum and urine M-protein levels, the |
| | difference between involved and uninvolved FLC levels (absolute increase |
| | must be >10 mg/dL); |
| | In patients without measurable serum and urine M-protein levels and witho [symbol] |
| | measurable involved FLC levels, BM plasma-cell percentage irrespective o [symbol] |
| | baseline status (absolute increase must be ≥10%); |
| | Appearance of a new lesion(s), ≥50% increase from nadir in SPD⁵ of >l |
| | lesion, or |
| | ≥50% increase in the longest diameter of a previous lesion >1 cm in short |
| | axis; |
| | ≥50% increase in circulating plasma cells (≥200 cells/μL) if this is the only |
| | measure of disease. |
| Clinical Relapse | Clinical relapse requires ≥1 of the following criteria: |
| | Direct indicators of increasing disease and/or end organ dysfunction (CRAB |
| | features) related to the underlying clonal plasma-cell proliferative disorder. It |
| | is not used in calculation of time to progression or PFS but is listed as |
| | something that can be reported optionally or for use in clinical practice; |
| | Development of new soft tissue plasmacytomas or bone lesions (osteoporotic |
| | fractures do not constitute progression); |
| | Definite increase in the size of existing plasmacytomas or bone lesions. |
| | Definite increase is defined as a 50% (and ≥1 cm) increase as measured |
| | serially by the SPD5 of the measurable lesion; |
| | Hypercalcemia (>11 mg/dL); |
| | Decrease in hemoglobin of ≥2 g/dL not related to therapy or other non- |
| | myeloma-related conditions; |
| | Rise in serum creatinine by ≥2 mg/dL from the start of the therapy and |
| | attributable to myeloma; |
| | Hyperviscosity related to serum paraprotein. |
| Relapse from CR | Any one or more of the following criteria: |
| (to be used only | Reappearance of serum or urine |
| if endpoint | M-protein by immunofixation or |
| is disease-free | electrophoresis; Development |
| survival) | of ≥5% plasma cells in the BM; |
| | Appearance of any other sign of |
| | progression (i.e., new plasmacytoma, lytic |
| | bone lesion, or hypercalcemia; see above). |
| Relapse from | Any one or more of the following criteria: |
| MRD-Negative | Loss of MRD-negative state (evidence |
| (to be used only | of clonal plasma cells on NGF or NG [symbol] |
| if endpoint | or positive imaging study |
| is disease-free | for recurrence of myeloma); |
| survival) | Reappearance of serum or urine M-protein by immunofixation or |
| | electrophoresis; Development of ≥5% clonal plasma cells in the BM; |
| | Appearance of any other sign of progression (i.e., new plasmacytoma, lytic |
| | bone lesion, or hypercalcemia). |

BM: bone marrow;
CR: complete response;
CRAB: calcium elevation, renal failure, anemia, lytic bone lesions;
CT: computed tomography;
FLC: free light chain;
h: hour;
IMWG: International Myeloma Working Group;
M-protein: monoclonal protein;
MR: minimal response;
MRD: minimal residual disease;
MRI: magnetic resonance imaging;
NGF: next-generation flow;
NGS: next-generation sequencing;
PD: progressive disease;
PET: positron emission tomography;
PFS: progression-free survival;
PR: partial response;
sCR: stringent complete response;
SD: stable disease;
SPD: sum of products of maximal perpendicular diameters of measured lesions;
VGPR: very good partial response.
¹Derived from international uniform response criteria for multiple myeloma (Durie et al., 2006). Minor response definition and clarifications derived from Rajkumar and colleagues (Rajkumar et al., 2011). When the only method to measure disease is by serum FLC levels: CR can be defined as a normal FLC ratio of 0-26 to 1-65 in addition to the CR criteria listed previously. VGPR in such patients requires ≥90% decrease in difference between involved and uninvolved FLC levels. All response categories require 2 consecutive assessments made at any time before institution of any new therapy; all categories also require no known evidence of progressive or new bone lesions or extramedullary plasmacytomas if radiographic studies were performed. Radiographic studies are not required to satisfy these response requirements. BM assessments do not need to be confirmed. Each category, except for SD, may be considered unconfirmed until the confirmatory test is performed. Date of initial test is considered as date of response for evaluation of time-dependent outcomes such as duration of response.
²All recommendations regarding clinical uses relating to serum FLC levels or FLC ratio are based on results obtained with the validated Freelite test (Binding Site, Birmingham, UK).
³Presence/absence of clonal cells on immunohistochemistry is based on the κ/λ/L ratio. An abnormal κ/λ ratio by immunohistochemistry requires ≥100 plasma cells for analysis. An abnormal ratio reflecting presence of an abnormal clone is κ/λ of >4:1 or <1:2.
4Special attention should be given to the emergence of a different monoclonal protein following treatment, especially in the setting of patients having achieved a conventional CR, often related to oligoclonal reconstitution of the immune system. These bands typically disappear over time and in some studies have been associated with a better outcome. Also, appearance of monoclonal IgG κ in patients receiving monoclonal antibodies should be differentiated from the therapeutic antibody.
⁵Plasmacytoma measurements should be taken from the CT portion of the PET/CT, or MRI scans, or dedicated CT scans where applicable. For patients with only skin involvement, skin lesions should be measured with a ruler. Measurement of tumor size may be determined by SPD.
⁶Positive immunofixation alone in a patient previously classified as achieving CR may not be considered progression. For purposes of calculating time to progression and PFS, patients who have achieved CR and are MRD-negative should be evaluated using criteria listed for PD. Criteria for relapse from CR or relapse from MRD should be used only when calculating disease-free survival.
⁷In the case in which a value is felt to be a spurious result per physician discretion (e.g., possible laboratory error), that value may not be considered when determining the lowest value. [symbol]
indicates data missing or illegible when filed

TABLE 23
IMWG Minimal Residual Disease Criteria

| MRD Status¹ | Description |
|---|---|
| Sustained MRD-Negative | MRD negativity in BM (NGF and/or NGS) and by imaging as defined below |
| | confirmed ≥1 year apart. Subsequent evaluations can be used to further |
| | specify the duration of negativity (e.g., MRD-negative at 5 years).² |
| Flow MRD-Negative | Absence of phenotypically aberrant clonal plasma cells by NGF³ on BM |
| | aspirates using the EuroFlow standard operation procedure for MRD detectio [symbol] |
| | in multiple myeloma (or validated equivalent method) with sensitivity of ≥1 |
| | 10⁵ nucleated cells. |
| Sequencing MRD-Negative | Absence of clonal plasma cells by NGS on BM aspirate in which presence of |
| | a clone is defined as <2 identical sequencing reads obtained after DNA |
| | sequencing of BM aspirates using the LymphoSIGHT platform (or validated |
| | equivalent method) with sensitivity of ≥1 in 10⁵ nucleated cells.⁴ |
| Imaging + MRD-Negative | MRD negativity, as defined by NGF or NGS plus disappearance of every are |
| | of increased tracer uptake found at baseline or a preceding PET/CT or |
| | decrease to less mediastinal blood pool SUV or decrease to less than that of |
| | surrounding normal tissue.⁵ |

ASCT: autologous stem cell transplant;
BM: bone marrow;
CT: computed tomography;
FDG: ¹⁸F-fluorodeoxyglucose;
IMWG: International Myeloma Working Group;
MFC: multiparameter flow cytometry;
MRD: minimal residual disease;
NGF: next-generation flow;
NGS: next-generation sequencing;
PET: positron emission tomography;
SUV: standard update value;
SUVmax: maximum standardized uptake value.
Note:
For MRD assessment, first BM aspirate should be sent to MRD (not for morphology) and this sample should be taken in 1 draw with a volume of ≥2 mL (to obtain sufficient cells), but maximally 4-5 mL to avoid hemodilution.
¹For MRD there is no need for 2 consecutive assessments. MRD tests should be initiated only at the time of suspected complete response. All categories of MRD require no known evidence of progressive or new bone lesions if radiographic studies were performed. However, radiographic studies are not required to satisfy these response requirements except for the requirement of FDG PET if imaging MRD-negative status is reported.
²Sustained MRD negativity when reported should also annotate method used (e.g., sustained flow MRD-negative, sustained sequencing MRD-negative).
³Bone marrow MFC should follow NGF guidelines (Paiva et al., 2012). The reference NGF method is an 8-color 2-tube approach that has been extensively validated. 5 million cells should be assessed. The flow cytometry method employed should have a sensitivity of detection of ≥1 in 10⁵ plasma cells.
⁴DNA sequencing assay on BM aspirate should use a validated assay such as LymphoSIGHT (Sequenta).
⁵Criteria used by Zamagni and colleagues (Zamagni et al., 2015), and expert panel (IMPetUs; Italian Myeloma criteria for PET Use) (Nanni et al., 2016; Usmani et al., 2013). Baseline positive lesions were identified by presence of focal areas of increased uptake within bones, with or without any underlying lesion identified by CT and present on ≥2 consecutive slices. Alternatively, an SUVmax = 2.5 within osteolytic CT areas >1 cm in size, or SUVmax =1.5 within osteolytic CT areas ≤1 cm in size were considered positive. Imaging should be performed once MRD negativity is determined by MFC or NGS. [symbol]
indicates data missing or illegible when filed

TABLE 24
Required Baseline and Follow-up Tests for Response
Assessment Using IMWG Response Criteria

| | Every | | At | At |
|---|---|---|---|---|
| | Response | No | Sus- | Sus- |
| | Assess- | Measurable | pected | pected |
| Test | ment | Protein ¹ | C [symbol] | D [symbol] 2 |
| SPEP (serum M-spike ≥1 g/dL | | — | X | X |
| Serum immunofixation (any) | | X | X | X |
| UPEP (urine M-spike | | — | X | X |
| ≥200 mg/24 h) | | | | |
| Urine immunofixation (any) | | X | X | — |
| Serum FLC | | — | X | X |
| Serum M-spike <1 g/dL, urine | | | | |
| M-spike <200 mg/24 h, | | | | |
| but involved | | | | |
| Ig FLC ≥10 mg/dL | | | | |
| Any | | — | X | X |
| Bone marrow aspirate/biopsy | X | — | X | — |
| Serum M-spike, urine M-spike | | | | |
| or involved Ig FLC not | | | | |
| meeting above criteria but BN [symbol] | | | | |
| plasma cell percentage ≥30% | | | | |
| Any | | — | X | — |
| Plasmacytoma (PET imaging) | X | — | X | — |
| Serum M-spike, u rine M-spike | | | | |
| involved Ig FLC or BM not | | | | |
| meeting above criteria, but ≥ [symbol] | | | | |
| lesion with single diameter of | | | | |
| ≥2 cm | | | | |
| Any | | — | X | — |
| Hemoglobin, serum calcium, | | — | — | X |
| creatinine (any) | | | | |

BM: bone marrow;
CR: complete response;
DP: disease progression;
FLC: free light chain;
h: hour;
Ig: immunoglobulin;
IMWG: International Myeloma Working Group;
M-spike: spike in monoclonal protein;
PET: positron emission tomography;
SPEP: serum protein electrophoresis;
UPEP: urine protein electrophoresis.
¹By electrophoresis.
2Clinical or biochemical.
³Baseline M-spike of ≥0.5 g/dL acceptable if very good partial response or higher is the response endpoint to be measured, and if progression-free survival or time to progression are endpoints of interest.
⁴To be done at assessment time point or complete response, or as clinically indicated, and then at suspected progression [symbol]
indicates data missing or illegible when filed

Monoclonal Protein Measurements in Serum and Urine

Blood and 24-hour urine samples for M-protein measurements may be sent to and analyzed by a central laboratory, and reviewed by the IRC for efficacy analyses per the schedule in Table and Table 19, and as clinically indicated. Serum and 24-hour urine samples may be collected for each time point and the following tests performed by a central laboratory:
Serum M-protein quantitation by electrophoresis (SPEP)
Serum immunofixation
Serum free light chain assay (FLC, kappa and lambda)
24-hour urine M-protein quantitation by electrophoresis (UPEP). Note: For screening, 24-hour urine collection may begin the day before informed consent.
Urine immunofixation
Quantitative immunoglobulins (Ig), if needed (e.g., IgA or IgD myeloma)
   In addition to central lab testing, serum and urine M-protein assessments may be performed locally and used for determination of study eligibility and clinical decisions regarding patient care. For screening, prior laboratory values (MM serum and urine results) obtained locally within 2 weeks of screening may be used provided that they were not associated with prior anticancer treatment (at least 2 weeks from last dose of anticancer therapy or at time of disease progression while on therapy).

Whole Body PET/CT Radiographic Disease Assessment

Baseline whole body (vertex to toes) PET/CT is to be performed at screening (i.e., within days prior to CTX120 infusion) and upon suspected CR. For subjects with evidence of extramedullary disease (e.g., extramedullary plasmacytoma or myelomatous lesion with soft tissue involvement), postinfusion scans may be conducted per the schedule of assessments in Table 18 and Table 19, per IMWG response criteria (Appendix A), and as clinically indicated. In subjects with extramedullary disease, the CT portion of PET/CT should be of diagnostic quality (e.g., CT with IV contrast) sufficient for tumor size measurement. MRI with contrast may be used for the CT portion when CT is clinically contraindicated or as required by local regulation.

PET/CT (with IV contrast) obtained as part of standard of care within 4 weeks prior to subject enrollment may be used to satisfy screening requirements.

Requirements for the acquisition, processing, and transfer of scans are outlined in the Imaging Manual. Whenever possible, the imaging modalities, machines, and scanning parameters used for radiographic disease assessment should be kept consistent during the study. For efficacy analyses, radiographic disease assessments may be performed by the IRC in accordance with IMWG response criteria.

Bone Marrow Aspirate and Biopsy

Bone marrow aspirate and biopsy are performed according to the schedule of assessments in Table 18 and Table 19, and as clinically indicated. Bone marrow aspirate/biopsy on Day 14 is optional and requires specific consent. Bone marrow sample collection (aspirate and biopsy) at screening should be performed during the 14-day screening period. Upon consultation with and agreement from the medical monitor, bone marrow biopsy obtained as part of standard of care within 4 weeks prior to subject enrollment may be used to satisfy screening requirements. All other bone marrow sample collection should be performed ±5 days of visit date. Standard institutional guidelines for the bone marrow biopsy should be followed.

Percentage of plasma cells is assessed on bone marrow aspirate and biopsy samples by a central laboratory and reviewed by the IRC as part of disease response evaluation per IMWG response criteria. For subjects who achieve suspected CR, a bone marrow biopsy to confirm response assessment by immunohistochemistry and MRD evaluation (on bone marrow aspirate) may be performed by a central laboratory. At any point that bone marrow collection is performed, aspirate samples should also be sent to a central laboratory for measurement of CTX120 and/or other exploratory analyses.

Extramedullary Plasmacytoma Biopsy

At progression, biopsy of extramedullary plasmacytoma, if present, should be collected (if medically feasible) to confirm disease (local testing) and for biomarker analysis (central testing). For subjects with extramedullary disease, tumor biopsy is also encouraged at screening and at least 1 post-CTX120 infusion timepoint.

Beta-2 Microglobulin and Cytogenetics

A serum sample to assess B2M level are obtained at screening and sent to a local laboratory for analysis. A bone marrow sample to evaluate cytogenetics (karyotyping and fluorescence in situ hybridization) should be performed at screening only and assessed locally (Table 18).

6.2.8. Laboratory Tests

Laboratory samples may be collected and analyzed according to the schedule of assessment (Table 18 and Table 19). Local laboratories meeting Clinical Laboratory Improvement Amendments requirements may be utilized to analyze all tests listed in Table 25 according to standard institutional procedures.

TABLE 25
Local Laboratory Tests

| | |
|---|---|
| CBC with differential | Hematocrit, hemoglobin, red blood cell count, white blood cell count, |
| | neutrophils, lymphocytes, monocytes, basophils, eosinophils, platelet count |
| | ANC |
| Lymphocyte subsets | 6-color TBNK panel or equivalent (T cells, B cells, and NK cells; see |
| | Laboratory Manual for additional instructions) |
| Serum chemistry¹ | ALT (SGPT), AST (SGOT), bilirubin (total and direct), albumin, alkaline |
| | phosphatase, bicarbonate, blood urea nitrogen, calcium, chloride, creatinine |
| | eGFR, glucose, lactate dehydrogenase, magnesium, phosphorus, potassium |
| | sodium, total protein |
| Coagulation | Prothrombin time, activated partial thromboplastin time, international |
| | normalized ratio, fibrinogen |
| Viral serology | HIV-1, HIV-2, hepatitis C virus antibody and PCR, hepatitis B surface |
| | antigen, hepatitis B surface antibody, hepatitis B core antibody |
| Immunoglobulins | IgA, IgG, IgM |
| CRS/HLH monitoring | Ferritin, CRP |
| Serum or urine pregnancy² | Human chorionic gonadotropin |

ALT: alanine aminotransferase;
ANC: absolute neutrophil count;
AST: aspartate aminotransferase;
CBC: complete blood count;
CRP: C-reactive protein;
CRS: cytokine release syndrome;
eGFR: estimated glomerular filtration rate;
HIV-1/-2: human immunodeficiency virus type 1 or 2;
HLH: hemophagocytic lymphohistiocytosis;
IgA/G/M: immunoglobulin A, G, or M;
LD: lymphodepleting;
PCR: polymerase chain reaction;
NK: natural killer;
SGOT: serum glutamic oxaloacetic transaminase;
SGPT: serum glutamic pyruvic transaminase,
TBNK: T, B, and NK cells.
²For females of childbearing potential only. Serum pregnancy test required at screening. Serum or urine pregnancy test within 72 hours before start of LD chemotherapy.

6.3 Biomarkers

Blood, bone marrow, CSF samples (only in subjects with treatment-emergent neurotoxicity), and, if applicable, tumor biopsy of extramedullary plasmacytoma is collected to identify genomic, metabolic, and/or proteomic biomarkers that may be indicative of clinical response, resistance, safety, disease, pharmacodynamic activity, or the mechanism of action of CTX120. Samples may be collected and shipped for testing at a central laboratory.

6.3.1 Analysis of CTX120 Levels

Analysis of levels of transduced BCMA-directed CAR+ T cells is performed on blood samples collected according to the schedule described in Table 18 and Table 19. The time course of the disposition of CTX120 in blood may be described using a PCR assay that measures copies of CAR construct per μg DNA. Complementary analyses using flow cytometry to confirm the presence of CAR protein on the cellular surface may also be performed.

Samples for analysis of CTX120 levels should be sent to the central laboratory from any blood, bone marrow, CSF, or biopsy of extramedullary plasmacytoma performed following CTX120 infusion. If CRS occurs, samples for assessment of CTX120 levels should be collected every 48 hours between scheduled visits until CRS resolves. The trafficking of CTX120 in bone marrow, CSF, or extramedullary plasmacytoma tissue may be evaluated in any of these samples collected as per protocol-specific sampling.

6.3.2 Cytokines

Cytokines, including IL-1β, soluble IL-1 receptor alpha (sIL-1Rα), IL-2, sIL-2Rα, IL-4, IL-6, IL-8, IL-10, IL-12p70, IL-13, IL-15, IL-17a, interferon γ, tumor necrosis factor α, and GM-CSF, are analyzed. Correlational analysis performed in multiple prior CAR T cell clinical studies have identified these cytokines, and others, as potential predictive markers for severe CRS and/or neurotoxicity, as summarized in a recent review (Wang and Han, 2018). Blood for cytokines is collected at specified times as described in Table 18. In subjects experiencing signs or symptoms of CRS, additional samples should be drawn daily until resolution.

6.3.3 Anti-CTX120 Antibody

The CAR construct is composed of humanized scFv. Blood is collected throughout the study to assess for potential immunogenicity, per Table 18 and Table 19.

6.3.4 Exploratory Research Biomarkers Exploratory research may be conducted to identify molecular (genomic, metabolic, and/or proteomic) biomarkers and immunophenotypes that may be indicative or predictive of clinical response, resistance, safety, disease, pharmacodynamic activity, and/or the mechanism of action of treatment. Samples may be collected according to the schedule in Table 18. Samples for exploratory biomarkers should also be sent for analysis from any lumbar puncture or BM sample collection (aspirate/biopsy) performed following CTX120 infusion. In the event of CRS, samples for exploratory biomarker assessment may be collected every 48 hours between scheduled visits until CRS resolves. Refer to the Laboratory Manual for instructions on collection of blood, bone marrow, extramedullary plasmacytoma, and CSF samples to support exploratory research.

### 7. Safety and Adverse Events

AEs in response to a query, observed by site personnel, or reported spontaneously by the subject are recorded. All AEs are followed to a satisfactory conclusion.

7.1 Adverse Events

An AE is any untoward medical occurrence or deterioration of a pre-existing medical condition in a clinical trial subject to whom the investigational medicinal product is administered, which does not necessarily have a causal relationship with this treatment. In clinical studies, an AE can include an undesirable medical condition occurring at any time, including baseline or washout periods, even if no study treatment has been administered.

An example of an AE is a clinically significant worsening in the nature, severity, frequency, or duration of a pre-existing condition.

The term “disease progression,” as assessed by measurement of malignant lesions on radiographs or other methods, should not be reported as an AE, unless the progression of malignancy under study is considered to be atypical in its nature, presentation, or severity from the normal course of the disease. Worsening of signs and symptoms of the malignancy under study should be reported as AEs in the appropriate section of the case report form (CRF).

Interventions for pretreatment conditions (such as elective surgery) or medical procedures that were planned before study participation are not considered AEs. Hospitalization for study treatment infusions or precautionary measures (including hospitalization for observation after CTX120 infusion) per institutional policy are not considered AEs or SAEs. Furthermore, if a subject has a planned hospitalization following CTX120 infusion, prolongation of that hospitalization for observation alone should not be reported as an SAE unless it is associated with a medically significant event that meets other SAE criteria.

Abnormal laboratory findings that result in new or worsening clinical sequelae, require therapy, or adjustment in current therapy are considered AEs and should be graded and reported per CTCAE v5.0. Where applicable, clinical sequelae (not the laboratory abnormality) are to be recorded as the AE.

7.2 Serious Adverse Event

An AE of any untoward medical consequence must be classified as a serious adverse event if it meets any of the following criteria:
Results in death
Is life-threatening (i.e., an AE that places the subject at immediate risk of death)
Requires in-patient hospitalization or prolongs an existing hospitalization (hospitalizations for scheduled medical or surgical procedures or to conduct scheduled observation and treatments do not meet these criteria)
Results in persistent or significant disability or incapacity
Results in a congenital anomaly or birth defect in the newborn
The AE is considered to be an event that may jeopardize the subject (i.e., puts the subject at risk) and may require medical or surgical intervention (treatment) to prevent one of the outcomes above.

7.3 Adverse Events of Special Interest

Based on the reported clinical experience of autologous CAR T cells, adverse events of special interest (AESIs) can be identified. AESIs must be reported any time after CTX120 infusion and include:
CTX120 infusion reactions
Grade≥3 opportunistic/invasive infections
Grade≥3 tumor lysis syndrome
Cytokine release syndrome
ICANS
Hemophagocytic lymphohistiocytosis
Graft versus host disease
Secondary malignancy
Uncontrolled T cell proliferation

In addition to the AESIs listed above, any new hematological or autoimmune disorder that is determined to be possibly related or related to CTX120 should be reported any time after CTX120 infusion.

7.4 Adverse Event Severity

AEs are graded according to CTCAE v5.0, with the exception of CRS, neurotoxicity, and GvHD, which are graded according to the criteria provided herein.

When a CTCAE grade or protocol-specified criteria are not available, the toxicity grading in Table 26 can be used.

TABLE 26
Adverse Event Severity

| | |
|---|---|
| Grade 1 | Mild; asymptomatic or mild symptoms; clinical or diagnostic observations |
| | only; intervention not indicated. |
| Grade 2 | Moderate; minimal, local, or noninvasive intervention indicated; limiting ag [symbol] |
| | appropriate instrumental ADL.¹ |
| Grade 3 | Severe or medically significant but not immediately life-threatening; |
| | hospitalization or prolongation of hospitalization indicated; disabling; |
| | limiting self-care ADL.² |
| Grade 4 | Life-threatening consequences; urgent intervention indicated. |
| Grade 5 | Death related to AE. |

ADL: Activities of Daily Living;
AE: adverse event.
¹Instrumental ADL refer to preparing meals, shopping for groceries or clothes, using the telephone, managing money, etc.
²Self-care ADL refer to bathing, dressing and undressing, feeding self, using the toilet, taking medications, and not bedridden. [symbol]
indicates data missing or illegible when filed

7.5 Adverse Event Causality

The relationship between each AE and CTX120, LD chemotherapy is assessed, and any protocol-mandated study procedure (all assessed individually). The assessment of relationship is made.

Related: There is a clear causal relationship between the study treatment or procedure and the AE.

Possibly related: There is some evidence to suggest a causal relationship between the study treatment or procedure and the AE, but alternative potential causes also exist.

Not related: There is no evidence to suggest a causal relationship between the study treatment or procedure and the AE.

The following may be considered in the assessment: (1) the temporal association between the timing of the event and administration of the treatment or procedure, (2) a plausible biological mechanism, and (3) other potential causes of the event (e.g., concomitant therapy, underlying disease) when making their assessment of causality.

If an SAE is assessed to be not related to any study intervention, an alternative etiology must be provided in the CRF. If the relationship between the AE/SAE and the investigational product is determined to be “possible,” the event may be considered related to the investigational product for the purposes of expedited regulatory reporting.

7.6 Outcome

The outcome of an AE or SAE classified and reported as follows:
Fatal
Not recovered/not resolved
Recovered/resolved
Recovered/resolved with sequelae
Recovering/resolving
Unknown

7.7 Adverse Event Collection Period

The safety of all subjects enrolled in this study is recorded from the time of ICF signing until end of study; however, there are different reporting requirements for different time periods in the study. Table 27 describes the AEs that should be reported at each time period of the study. based on the following definitions:

TABLE 27
Adverse Event Collection by Study Time Period

| Time Period | AE Reporting Requirements |
|---|---|
| Informed consent to Month 3 study visit | All AEs |
| After Month 3 study visit to Month 60 visit | SAEs AESIs |
| After Month 3 study visit and subject starts a | CTX120-related SAEs |
| new anticancer therapy | CTX120-related AESIs |

AE: adverse event;
AESI: adverse event of special interest;
SAE: serious adverse event.

### 8. Termination Rules

The treatment is to be terminated if 1 or more of the following events occur:
Life-threatening (grade 4) toxicity attributable to CTX120 that is unmanageable and unexpected
Death related to CTX120 within 30 days of infusion
Grade≥3 GvH, for example,
   >35% grade 3 or 4 neurotoxicity not resolving within 7 days to grade≤2
   >20% grade≥2 GvHD that is steroid-refractory
   >30% grade 4 CRS
   >50% grade 4 neutropenia not resolving within 28 days (except for subjects with baseline neutropenia)
   >30% grade 4 infections
New malignancy (distinct from recurrence/progression of previously-treated malignancy)
Lack of efficacy, defined as 2 or fewer responses (including PR+VGPR+CR+stringent complete response [sCR]) after 15 subjects in dose expansion have 3 months of post-CTX120 assessment

### 9. Statistical Analyses

The primary objective of Part A is to assess the safety of escalating doses of CTX120 in subjects with relapsed or refractory multiple myeloma to determine the MTD and/or recommended dose for Part B cohort expansion.

The primary objective of Part B is to assess the efficacy of CTX120 in subjects with relapsed or refractory multiple myeloma, as measured by ORR according to IMWG response criteria.

9.1 Study Endpoints

9.1.1 Primary Endpoints

Part A (Dose Escalation): Incidence of adverse events defined as dose-limiting toxicities, and definition of the recommended dose for Part B cohort expansion

Part B (Cohort Expansion): Objective response rate (sCR+CR+VGPR+PR), per IMWG response criteria

9.1.2 Part A and B Secondary Endpoints

Efficacy:
Percentage of subjects with stringent complete response, per IMWG response criteria (Table 22)
Percentage of subjects with complete response, per IMWG response criteria (Table 22)
Percentage of subjects with very good partial response, per IMWG response criteria (Table 22)
Duration of response (DOR) may be reported only for subjects who have had sCR/CR/VGPR/PR events. This may be calculated as the time between first objective response of sCR/CR/VGPR/PR and date of disease progression by IMWG response criteria or death due to any cause.
Progression-free survival (PFS) may be calculated as the difference between date of CTX120 infusion and date of disease progression or death due to any cause. Subjects who have not progressed and are still on study at the data cutoff date may be censored at their last MM disease assessment date.
Overall survival (OS) may be calculated as the time between date of CTX120 infusion and death due to any cause. Subjects who are alive at the data cutoff date may be censored at their last date known to be alive.

Safety

Incidence and severity of adverse events and clinically significant laboratory abnormalities may be summarized and reported according to CTCAE v5.0, except for CRS, which may be graded according to Lee criteria (Lee et al., 2014) in Part A and ASTCT criteria (Lee et al., 2019) in Part B; neurotoxicity, which may be graded according to ICANS (Lee et al., 2019) and CTCAE v5.0; and GvHD, which may be graded according to MAGIC criteria (Harris et al., 2016).

Pharmacokinetics

The levels of CTX120 in blood and other tissues over time may be assessed using a PCR assay that measures copies of CAR construct per μg DNA. Complementary analyses using flow cytometry to confirm the presence of CAR protein on the cellular surface may also be performed.

The trafficking of CTX120 in bone marrow, CSF, or extramedullary plasmacytoma tissues may be evaluated in any of these samples collected as per protocol-specific sampling.

9.1.3 Part A and B Exploratory Endpoints
Levels of cytokines in blood and other tissues
Incidence of anti-CTX120 antibodies
Impact of anti-cytokine therapy on CTX120 proliferation, CRS, and disease response
Time to response, defined as the time between the date of CTX120 infusion until first documented response (sCR/CR/VGPR/PR)
Time to CR, defined as the time between the date of CTX120 infusion until first documented CR
Time to disease progression, defined as time between the date of CTX120 infusion until first evidence of disease progression
Percentage of subjects who are MRD-negative
Incidence of autologous or allogeneic SCT following CTX120 therapy
Incidence and type of subsequent anticancer therapy
Change from baseline in subject-reported health status, as measured by EORTC QLQ-30 and QLQ-MY20, and EQ-5D-5L questionnaires
First subsequent therapy-free survival, defined as the time between date of CTX120 infusion and date of first subsequent therapy or death due to any cause
Other exploratory genomic, proteomic, metabolic, or pharmacodynamic endpoints

9.2 Method of Analyses

The primary endpoint of ORR for all analyses (futility and primary) is based on central review of MM disease assessments provided herein in the FAS. Sensitivity analyses of ORR are also performed. Tabulations are to be produced for appropriate demographic, baseline, efficacy, and safety parameters. ORR may be summarized as a proportion with exact 95% confidence intervals, and an exact binomial test may be used to compare the observed response rate to an historical response rate of 30%. For time-to-event variables such DOR, PFS, and OS, medians with 95% confidence intervals may be calculated using Kaplan-Meier methods.

All subjects who receive CTX120 are included in the SAS. AEs are graded according to CTCAE v5.0, except for CRS (Lee criteria for Part A, ASTCT criteria for Part B), neurotoxicity (ICANS and CTCAE v5.0), and GvHD (MAGIC criteria). AEs, SAEs, and AESIs may be summarized by dose cohort and reported according to the following intervals:

Signing of ICF until 3 months after infusion: All AEs
After Month 3 visit until Month 60 visit: All SAEs and AESIs
After Month 3 study visit and subject starts a new anticancer therapy: CTX120-related SAEs and CTX120-related AESIs

Levels of CTX120 CAR+ T cells in blood, incidence of anti-CTX120 antibodies, and levels of cytokines in serum may be summarized. Investigation of additional biomarkers may include assessment of blood components (serum, plasma, and cells), cells from other tissues, extramedullary plasmacytoma tissue, and other subject-derived tissue. These assessments may evaluate DNA, RNA, proteins, and other biologic molecules derived from those tissues. Such evaluations may inform understanding of factors related to the subjects' disease, response to CTX120, and the mechanism of action of the investigational product.

### Results

To date, all subjects that participated in this study have completed Stage 1 (eligibility screening) within 16 days, with the majority of subjects completing this stage in less than 10 days. Upon confirmation of eligibility (i.e. enrollment), all eligible subjects have started lymphodepleting (LD) chemotherapy within 7 days, with over 75% of these subjects starting LD chemotherapy within 2 days. In addition, all eligible subjects have received CTX120 in less than 14 days, with the majority receiving CTX120 within 8 days of enrollment. All subjects receiving LD chemotherapy have progressed to receiving CTX120 within 2-7 days from completion of the LD chemotherapy; all but 1 subject received CTX120 within 4 days of completing LD chemotherapy. The enrolled subjects have relapsed and/or refractory multiple myeloma.

Two enrolled subjects had absolute neutrophil counts (ANC) below 1000 cells/mm³, and one of these subjects also had a platelet count of 28,000 cells/mm³ at the time of screening. These blood cell counts would likely exclude them from autologous CAR T therapies that typically impose hematologic eligibility criteria (e.g., ANC≥1000 cells/mm³; platelets≥50,000 cells/mm³)

None of the treated patients exhibited any DLTs. CTX120 has been detected in all subjects treated with the CAR T product. The allogeneic CAR-T cell therapy exhibited desired pharmacokinetic features in the treated human subjects, including CAR-T cell expansion and persistence after infusion. A dose dependent effect has been observed in both CTX120 expansion and persistence. CTX120 cells were detected in peripheral blood up to the latest time point tested (28 days post CAR-T dosing), with peak expansion detected 1-2 weeks post dosing. Expansion following dosing appeared dose dependent, with maximum expansion observed from 0 CAR copies/ug at nadir up to over 300 CAR copies/ug at peak.

A dose dependent response has been observed. For example, at DL2, evidence of anti-tumor response was observed in two subjects. These subjects showed decreases in serum/urine monoclonal protein, serum free light chain, and/or bone marrow plasma cells.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

1. A method for treating multiple myeloma (MM), the method comprising:
(i) administering to a subject in need thereof an effective amount of one or more lymphodepleting chemotherapeutic agents; and
(ii) administering to the subject an effective amount of a population of genetically engineered T cells after step (i);
wherein the population of genetically engineered T cells comprise T cells, which comprise a nucleic acid comprising a nucleotide sequence encoding a chimeric antigen receptor (CAR) that binds BCMA, a disrupted TRAC gene, and a disrupted β2M gene; and wherein the nucleic acid encoding the CAR is inserted into the disrupted TRAC gene.

2. The method of claim 1, wherein the CAR that binds BCMA comprises:
(i) an ectodomain comprising an anti-BCMA single chain variable fragment (scFv);
(ii) a CD8a transmembrane domain; and
(iii) an endodomain comprising a co-stimulatory domain from 4-1BB and a CD3ζ signaling domain.

3. The method of claim 2, wherein the anti-BCMA scFv comprises a heavy chain variable domain (V_{H}) comprising SEQ ID NO:42 and a light chain variable domain (V_{L}) comprising SEQ ID NO:43.

4. The method of claim 3, wherein the anti-BCMA scFv comprises SEQ ID NO: 41.

5. The method of claim 1, wherein the CAR that binds BCMA comprises the amino acid sequence of SEQ ID NO: 40.

6. The method of claim 5, wherein the nucleic acid encoding the anti-BCMA CAR comprises the nucleotide sequence of SEQ ID NO: 33.

7. The method of claim 1, wherein the disrupted TRAC gene is produced by a CRISPR/Cas9 gene editing system, which comprises a guide RNA comprising a spacer sequence of SEQ ID NO: 4.

8. The method of claim 1, wherein the disrupted TRAC gene has a deletion comprising SEQ ID NO:10, optionally wherein the disrupted TRAC gene comprises the nucleotide sequence of SEQ ID NO: 30, which substitutes for the deletion.

9. The method of claim 1, wherein the disrupted β2M gene is produced by a CRISPR/Cas9 gene editing system, which comprises a guide RNA comprising a spacer sequence of SEQ ID NO: 8.

10. The method of claim 1, wherein the disrupted β2M gene comprises at least one of SEQ ID NOs: 21-26.

11. The method of claim 1, wherein in the population of genetically engineered T cells, ≥30% of the genetically engineered T cells are CAR⁺, ≤0.4% of the genetically engineered T cells are TCR⁺, and/or ≤30% of the genetically engineered T cells are B2M⁺.

12. The method of claim 1, wherein the population of genetically engineered T cells is derived from one or more healthy human donors.

13. The method of claim 1, wherein the population of genetically engineered T cells is suspended in a cryopreservation solution.

14. The method of claim 1, wherein the effective amount of the population of genetically engineered T cells ranges from about 5.0×10⁷ to about 7.5×10⁸ CAR⁺ T cells, optionally wherein the effective amount of the population of genetically engineered T cells ranges from about 5.0×10⁷ to about 1.5×10⁸ CAR⁺ T cells, about 1.5×10⁸ to about 4.5×10⁸ CAR⁺ T cells, about 4.5×10⁸ to about 6.0×10⁸ CAR⁺ T cells, or about 6.0×10⁸ to about 7.5×10⁸ CAR⁺ T cells.

15. The method of claim 14, wherein the effective amount of the population of genetically engineered T cells is about 5.0×10⁷ CAR⁺ T cells, about 1.5×10⁸ CAR⁺ T cells, about 4.5×10⁸ CAR⁺ T cells, about 6.0×10⁸ CAR⁺ T cells, or about 7.5×10⁸ CAR⁺ T cells.

16. The method of claim 1, wherein the population of genetically engineered T cells is administered by intravenous infusion.

17. The method of claim 1, wherein step (i) comprises co-administering to the subject fludarabine at about 30 mg/m² and cyclophosphamide at about 300 mg/m² intravenously per day for three days.

18. The method of claim 1, wherein step (i) comprises co-administering to the subject fludarabine at about 30 mg/m² and cyclophosphamide at about 500 mg/m² intravenously per day for three days.

19. The method of claim 1, wherein step (ii) is performed 2-7 days after step (i).

20. The method of claim 1, wherein prior to step (i), the human patient does not show one or more of the following features:
(a) significant worsening of clinical status,
(b) requirement for supplemental oxygen to maintain a saturation level of greater than about 91%,
(c) uncontrolled cardiac arrhythmia,
(d) hypotension requiring vasopressor support,
(e) active infection, and
(f) neurological toxicity that increases risk of immune effector cell-associated neurotoxicity syndrome (ICANS).

21. The method of claim 1, wherein prior to step (ii) and after step (i), the human patient does not show one or more of the following features:
(a) active uncontrolled infection,
(b) worsening of clinical status compared to the clinical status prior to step (i), and
(c) neurological toxicity that increases risk of immune effector cell-associated neurotoxicity syndrome (ICANS).

22. The method of claim 1, further comprising (iii) monitoring the human patient for development of acute toxicity after step (ii).

23. The method of claim 22, wherein acute toxicity comprises cytokine release syndrome (CRS), neurotoxicity, tumor lysis syndrome, hemophagocytic lymphohistiocytosis (HLH), Cytopenias, GvHD, hypotention, renal insufficiency, viral encephalitis, neutropenia, thrombocytopenia or a combination thereof.

24. The method of claim 22, wherein the subject is subject to toxicity management if development of toxicity is observed.

25. The method of claim 1, wherein the subject is a human patient, who optionally is 18 years of age or older.

26. The method of claim 1, wherein the subject has relapsed and/or refractory MM.

27. The method of claim 1, wherein the subject has undergone at least two prior therapies for MM.

28. The method of claim 27, wherein the at least two prior therapies comprise an immunomodulatory agent, a proteasome inhibitor, an anti-CD38 antibody, or a combination thereof.

29. The method of claim 28, wherein the subject is refractory to prior therapies comprising an immunomodulatory agent and a proteasome inhibitor.

30. The method of claim 28, wherein the subject is refractory to prior therapies comprising an immunomodulatory agent, a proteasome inhibitor, and an anti-CD38 antibody.

31. The method of claim 1, wherein the subject relapsed after an autologous stem cell transplant (SCT), and wherein optionally the relapse occurs within 12 months after the SCT.

32. The method of claim 1, wherein the subject is a human patient having one or more of the following features:
(a) Measurable disease,
(b) Eastern Cooperative Oncology Group performance status 0 or 1,
(c) adequate organ function,
(d) free of a prior allogeneic stem cell transplantation (SCT),
(e) free of autologous SCT within 60 days prior to step (i),
(f) free of plasma cell leukemia, non-secretory MM, Waldenstrom's macroglobulinemia, POEM syndrome, and/or amyloidosis with end organ involvement and damage,
(g) free of contraindication to cyclophosphamide and/or fludarabine,
(h) free of prior gene therapy, anti-BCMA therapy, and non-palliative radiation therapy within 14 days prior to step (i),
(i) free of central nervous system involvement by MM,
(j) free of history or presence of clinically relevant CNS pathology, cerebrovascular ischemia and/or hemorrhage, dementia, a cerebellar disease, an autoimmune disease with CNS involvement,
(k) free of unstable angina, arrhythmia, and/or myocardial infarction within 6 month prior to step (i),
(l) free of uncontrolled infections, optionally wherein the infection is caused by HIV, HBV, or HCV,
(m) free of previous or concurrent malignancy, provided that the malignancy is not basal cell or squamous cell skin carcinoma, adequately resected and in situ carcinoma of cervix, or a previous malignancy that was completely resected and has been in remission for ≥5 years,
(n) free of live vaccine administration within 28 days prior to step (i),
(o) free of systemic anti-tumor therapy within 14 days prior to step (i), and
(p) free of primary immunodeficiency disorders or autoimmune disorders that require immunosuppressive therapy.

33. The method of claim 1, wherein the effective amount of the population of genetically engineered T cells is sufficient to achieve one or more of the following:
(a) decrease soft tissue plasmacytomas sizes (SPD) by at least 50% in the subject;
(b) decrease serum M-protein levels by at least 25%, optionally by 50% in the subject;
(c) decrease 24-hour urine M-protein levels by at least 50%, optionally by 90% in the subject;
(d) decrease differences between involved and uninvolved free light chain (FLC) levels by at least 50% in the subject;
(e) decrease plasma cell counts by at least 50% in the subject;
(f) decrease kappa-to-lambda light chain ratios (κ/λ ratios) to 4:1 or lower in the subject, who has myeloma cells that produce kappa light chains;
(g) increase kappa-to-lambda light chain ratios (κ/λ ratios) to 1:2 or higher in the subject, who has myeloma cells that produce lamda light chains.

34. The method of claim 1, wherein the effective amount of the population of genetically engineered T cells is sufficient to decrease serum M-protein levels by at least 90% and 24-hour urine M-protein levels to less than 100 mg in the subject, and/or wherein the effective amount of the population of genetically engineered T cells is sufficient to decrease serum M-proteins, urine M-proteins, and soft tissue plasmacytomas to undetectable levels, and plasma cell counts to less than 5% of bone marrow (BM) aspirates in the subject.

35. The method of claim 1, wherein the effective amount of the population of genetically engineered T cells is sufficient to achieve Stringent Complete Response (sCR), Complete Response (CR), Very Good Partial Response (VGPR), Partial Response (PR), Minimal Response (MR), or Stable Disease (SD).

36. A population of genetically engineered T cells, which comprise T cells comprising a nucleic acid comprising a nucleotide sequence encoding a chimeric antigen receptor (CAR) that binds BCMA, a disrupted TRAC gene, and a disrupted β2M gene; and wherein the nucleotide sequence encoding the CAR is inserted into the disrupted TRAC gene;
wherein in the population of genetically engineered T cells, ≥30% of the genetically engineered T cells are CAR⁺, ≤0.4% of the genetically engineered T cells are TCR⁺, and/or ≤30% of the genetically engineered T cells are B2M⁺.

37-51. (canceled)
